(19) United States
(12) Patent Application Publication
(10) Pub. No.: US 2002/0155479 A1
(43) Pub. Date: Oct. 24, 2002

(54) Expression cloning method

(75) Inventors: Jonathan S. Bogan, Belmont, MA (US); Harvey F. Lodish, Brookline, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA

(21) Appl. No.: 10/058,820

(22) Filed: Jan. 28, 2002

Related U.S. Application Data
(60) Provisional application No. 60/264,816, filed on Jan. 26, 2001.
(60) Provisional application No. 60/325,651, filed on Sep. 28, 2001.
(60) Provisional application No. 60/138,237, filed on Jun. 9, 1999.
(60) Provisional application No. 60/154,078, filed on Sep. 15, 1999.

(51) Int. Cl. C12Q 1/68; G01N 33/53; G01N 33/567; C12N 15/87

(52) U.S. Cl. 435/6; 435/455; 435/7.2; 435/325

(57) ABSTRACT

Described herein is a method of expression cloning useful for identifying and obtaining proteins involved in GLUT4 trafficking in mammalian cells and, thus, in insulin-stimulated glucose uptake by such cells. In particular, an enrichment strategy for expression cloning proteins involved in GLUT4 trafficking at the plasma membrane is described. Proteins identified by the method and uses therefore are also described.

### BACKGROUND OF THE INVENTION

The glucose transporter, GLUT4, is expressed predominantly in adipose and muscle tissues, where it accounts for the bulk of insulin-stimulated glucose uptake (Charron, M. J. et al., 1999, J Biol Chem 274:3253-6; Saltiel, A. R., 2001, Cell 104:517-529; Simpson, F. et al. 2001, Traffic 2:2-11). In the presence of insulin, GLUT4 is redistributed from an intracellular compartment to the plasma membrane, where it facilitates the diffusion of glucose into the cell (Cushman, S. W. and Wardzala, L. J., 1980, J Biol Chem 255:4758-62; Holman, G. D. and Cushman, S. W., 1996, Semin Cell Dev. Biol 7:259-268; Pessin, J. E. et al., 1999, J Biol Chem 274:2593-6; Rea, S. and James, D. E., 1997, Diabetes 46:1667-77; Suzuki, K. and Kono, T., 1980, Proc. Natl. Acad. Sci. U.S.A. 77:2542-5). Another glucose transporter isoform, GLUT1, is also expressed in fat and muscle tissues and is present at high levels in many other cell types and in cultured cell lines. A large proportion of GLUT1 is present on the plasma membrane even in the absence of insulin. Thus, while both GLUT1 and GLUT4 recycle at the plasma membrane, only GLUT4 recycling is characterized by significant intracellular sequestration, resulting from a slow rate of exocytosis, in the absence of insulin. Insulin increases the rate of GLUT4 exocytosis, with little or no decrease in its rate of endocytosis, so that in adipocytes the proportion of GLUT4 at the cell surface increases from <10% in the absence of insulin to 35-50% in its presence (Jhun, B. H. et al. 1992, J Biol Chem 267:17710-17715; Lee. W. et al. 1999, J Biol Chem 274:37755-62; Satoh, S. et al., 1993, J Biol Chem 268:17820-9; Yang., J and Holman, G. D., 1993, J Biol Chem 268:4600-3; Yeh, J. I. et al., 1995, Biochemistry 34:15523-31).

Characterization of the intracellular, insulin-responsive GLUT4-containing compartment is complicated by the fact that GLUT4 resides in several morphologically distinct locations within the cell. Ultrastructural studies have shown that GLUT4 is present in tubuvesicular structures distinct from lysosome, as well as in a perinuclear compartment that is in close vicinity to the trans-Golgi network (Hudson, A. W. et al., 1992, J Cell. Biol 116:785-97; Slot, J. W. et al., 1991, Proc. Natl. Acad. Sci. U.S.A. 88:7815-9; Slot, J. W. et al., 1991, J Cell Biol 1113:123-35; Smith, R. M. et al., 1991, Natl. Acad. Sci. U.S.A. 88:6893-7). Recent work demonstrates that approximately 40-45% of intracellular GLUT4 localizes in a transferring receptor (TfnR)-positive endosomal compartment, while 50-60% is in a second, TfnR-negative compartment; it is GLUT4 in this TfnR-negative compartment that is rapidly mobilized upon insulin addition (Aledo, J. C. et al., 1997, Biochem J, 325:727-32; Hashiramoto. M. and James. D. E., 2000, Mol Cell Biol 20:416-27; Kandror, K. V. and Pilch. P. F., 1998, Biochem J 331:829-35; Lee, W. et al., 1999, J Biol Chem 274:37755-62; Martin, S. J. et al., 1997, J Cell Sci 110:2281-2291; Ramm, G. et al, 2000, Mol Biol Cell 11:4079-91). In primary adipocytes, this TfnR-negative compartment can be subdivided into separate storage and exocytic pools of GLUT4 (Lee, W. et al, 1999, J Biol Chem 274:37755-62; Lee, W. et al., 2000, Biochemistry 39:9358-66). Other data suggest that the TfnR-positive GLUT4 compartment is the precursor of the TfnR-negative, insulin responsive compartment (Wei, M. L. et al., 1998, J Cell Biol 140:565-75). Moreover, targeting motifs within GLUT4 mediate its distribution between TfnR-negative and TfnR-positive compartments (Martin, S. et al., 1997, J Cell Sci. 110:2281-2291). Studies using the cation-dependent mannose 6-phosphate receptor (CD-M6PR) as an endosomal marker also find a similar, highly insulin-responsive, M6PR-negative pool of intracellular GLUT4 in 3T3-L1 adipocytes (Martin, S. et al., 2000, J Cell Sci. 113:3427-38). Thus it appears that GLUT4 is sequestered out of endosomes and into a highly insulin-responsive compartment. It has recently been shown that 3T3-L1 adipocytes also possess an insulin-regulated secretory compartment containing ACRP30, a TNFα-like protein produced exclusively by adipocytes (Bogan, J. S. and Lodish, H. F., 1999, J Cell Biol 146:609-20; Scherer, P. E. et al., 1995, J Biol Chem 270:26746-9; Shapiro, L. and Scherer, P. E., 1998, Curr. Biol 8:335-8). This regulated secretory compartment is distinct from the insulin-regulated compartment containing GLUT4 (Bogan, J. S. and Lodish, H. F., 1999, J Cell Biol 146:609-20).

Kinetic studies are consistent with the notion that there are multiple compartments through which GLUT4 traffics in adipocytes. GLUT4 recycles between the plasma membrane and intracellular sites in both basal and insulin-stimulated states, yet the initial externalization of GLUT4 after insulin addition is too rapid to be explained by the steady-state rate constants for exocytosis and endocytosis in the presence of insulin (Clark, A. E. et al., 1991, Biochem J 278:235-41; Satoh, S. et al., 1993, J Biol Chem 268:17820-9; Yang, J. and Holman, G. D., 1993, J Biol Chem 268:4600-3). It has therefore been argued that a 2-pool model, with one intracellular and one plasma membrane compartment, does not explain the observed kinetics of GLUT4 externalization after insulin addition, and that GLUT4 must traffic through three or more compartments (Holman, G. D. et al., 1994, J Biol Chem 269:17516-24; Satoh, S. et al., 1993, J Biol Chem 268:17820-9; Yeh, J. I. et al., 1995, Biochemistry 34:15523-31). Among these compartments is postulated to be a highly insulin-responsive, intracellular compartment from which GLUT4 is rapidly mobilized by insulin (Holman, G. D. et al., 1994, J Biol Chem 269:1751-24; Lee, W. et al., 2000, Biochemistry 39:9358-66; Yeh, J. I. et al., 1995, Biochemistry 34:15523-31). The GLUT4 accumulated in this highly insulin-responsive compartment in the basal state is depleted upon insulin addition, and the steady-state exocytosis rate for GLUT4 in the continued presence of insulin becomes limited at some other step in the recycling pathway. This kinetically defined, highly insulin-responsive compartment in adipocytes corresponds well with the morphologically and biochemically defined, TfnR-negative compartments containing GLUT4 in adipocytes and myocytes, which are depleted of GLUT4 after acute insulin stimulation (Aledo, J. C. et al., 1997, Biochem J 325:727-32; Hashiramoto, M. and James, D. E., 2000, Mol. Cell Biol 20:416-27; Kandror, K. V. and Pilch, P. F., 1998, Biochem J 331:829-35; Lee, W. J. et al., 1999, J Biol Chem 274:37755-62; Livingstone, C. et al., 1996, Biochem J 315; 487-95; Martin, S. et al., 1997, J Cell Sci. 110;2281-2291; Ramm, G. et al. 2000, Mol. Biol Cell 11:4079-91).

The mechanisms controlling GLUT4 accumulation in this specialized, insulin-sensitive compartment are poorly understood. The compartment is believed to be present only in muscle and fat, and apparently develops early during 3T3-L1 adipocyte differentiation in cell culture (Czech, M. P. et al., 1993, J Cell Biol 123:127-35; El-Jack, A. K. et al., 1999, Mol. Biol Cell 10:1581-1594; Haney, P. M. et al., 1991, J Cell Biol 114:689-99; Herman, G. A. et al., 1994, PNAS USA 91:12750-4; Hudson, A. W. et al., 1993, J Cell Biol 122:579-88; Hudson, A. W. et al., 1992, J Cell Biol 116; 785-97; Ross, S. A. et al., 1998, Biochem J 330:1003-8; Schunmann, A. et al., 1992 Biochem Biophys Acta 1131:245-52; Verhey, K. J. et al., 1993, J Cell Biol 123:137-47). Some data indicate that a similar compartment to which GLUT4 is targeted is present in CHO cells, though this has been controversial (Ishii, K. et al., 1995, Biochem J 309:813-23; Kanai, F. et al., 1993, J Biol Chem 268:14523-6; Shibasaki, Y. et al., 1992, Biochem J 281:829-34; Todaka, M. et al., 1996, Biochem J 315:875-82; Wei, M. et al., 1998, J Cell Biol 140:565-75). Kinetic studies suggest that even if some GLUT4 is targeted to a highly insulin-responsive compartment in CHO cells, sorting to this compartment is not efficient and does not constitute a major pathway by which GLUT4 traffics, in contrast to the case in adipocytes (Araki, S. et al., 1996, Biochem J 315:153-9). Studies of the insulin-responsive aminopeptidase (IRAP), a protein of uncertain physiologic function that is thought to cotraffic with GLUT4, show that this protein participates in similar mechanisms for dynamic retention in the endosomal systems of CHO cells and 3T3-L1 adipocytes (Johnson, A. O. et al., 1998, J Biol Chem 273:17968-77; Subtil, A. et al., 2000, J Biol Chem 275:4787-95). The assertion that the trafficking mechanisms employed by IRAP are identical to those used by GLUT4 rests upon evidence that these proteins colocalize and cotraffic, with similar kinetics, under all conditions. Most data support this hypothesis (Filippis, A. et al., 1998, Biochem J 330:405-11; Garza, L. A. et al., 2000, J Biol Chem 275:2560-7; Kandror, K. V., 1999, J Biol Chem 274:25210-25217; Malide, D. et al., 1997, FEBS Letters 409:461-468; Martin, S. et al., 1997, J Cell Sci. 110:2281-2291; Ross, S. A. et al., 1997, Biochem Biophys Res. Comm. 239:247-251; Sumitani, S. et al., 1997, Endocrinology 138:1029-34). Of note, trafficking of IRAP, like that of GLUT4, is much more insulin-responsive in 3T3-L1 adipocytes than in CHO cells (Johnson, A. O., et al., 2000, J Biol Chem 273:17968-77; Subtil, A., et al., J Biol Chem 275:4787-96). This is presumed to result from cell-type specific differences, though as with GLUT4, little is known about the mechanisms regulating IRAP accumulation and release from the highly insulin-responsive pool.

In conclusion, despite the fact that insulin-stimulated glucose uptake has been extensively studied, little is definitively known about the proteins involved in the process. It would be helpful to have additional information about this metabolic process and to identify additional proteins which might be involved.

### SUMMARY OF THE INVENTION

Described herein is a method of expression cloning useful for identifying and obtaining proteins involved GLUT4 trafficking of mammalian cells. In particular embodiments the method is useful for identifying and obtaining proteins involved in insulin stimulated GLUT4 trafficking at the plasma membrane of mammalian cells and, thus, in insulin-stimulated glucose uptake by such cells. In one embodiment, an enrichment strategy for expression cloning proteins involved in GLUT4 trafficking is described. The method comprises sorting cells containing an expression library comprising DNA encoding a protein involved in insulin stimulated GLUT 4 trafficking for cells with an altered proportion of GLUT4 at the cell surface. The method can further include expanding the sorted cells with an altered proportion of GLUT4 in culture, further sorting the expanded cells to identify cells with an altered proportion of GLUT4 and expanding the sorted expanded cells in culture, thereby forming an expression library enriched for DNA encoding a protein involved in GLUT4 trafficking at the plasma membrane.

In particular embodiments, the cells respond to insulin stimulation by externalizing GLUT4 in a biphasic pattern characterized by a first “peak” phase followed by a lowered “steady-state” phase. In embodiments, the cells with an altered proportion of GLUT4 at the plasma membrane are identified about 5 minutes after insulin stimulation. The proportion of GLUT4 at the cell surface can be increased. Alternatively, the proportion of GLUT4 at the cell surface can be decreased. The method of enriching the expression library can include further repetitions of the steps of sorting the expanded cells and expanding the cells in culture prior to identifying the clone of interest. Any number of repetitions are contemplated, but typically two, three, four or five additional repetitions are utilized. In a particular embodiment, the cells are sorted and individual cells are identified and clonally expanded. For example, individual cells can be placed in wells of 96-well titer plates and expanded. In one embodiment, the population of cells is cultured in a media with a high amino acid content such as Dulbecco's modified Eagle's medium (DMEM). In embodiments, the cells are adipocyte, fibroblasts or muscle cells, e.g., skeletal muscle cells. In particular embodiments, the cells are 3T3-L1 or Chinese Hamster Ovary (CHO) cells. After an enriched expression library is obtained a desired nucleic acid encoding a protein involved in GLUT4 trafficking can be excised and sequenced using known techniques.

In another aspect, the invention is directed to the expression library formed by the method. Such expression libraries are enriched in expression products encoding proteins involved in GLUT4 trafficking at the plasma membrane. In particular embodiments, the expression libraries contain proteins comprising UBX domains.

In another aspect, the invention is directed to a method for identifying a protein. In embodiments, such proteins are involved in insulin stimulated GLUT4 trafficking at the plasma membrane. The method comprises preparing an expression library enriched for DNA encoding a protein involved in GLUT4 trafficking at the plasma membrane in cells, stimulating the cells with insulin, and screening the cells, thereby identifying a protein involved in GLUT4 trafficking at the plasma membrane. In embodiments, the cells respond to insulin stimulation by externalizing GLUT4 in a biphasic pattern. In embodiments, the cells are selected by identifying those cells with altered proportions of GLUT4 at the cell surface about five minutes after insulin stimulation. The expression library can be enriched for DNA encoding proteins associated with an increased proportion of GLUT4 at the plasma membrane, or alternatively, the expression library can be enriched for DNA encoding proteins associated with a decreased proportion of GLUT4 at the plasma membrane. The cells can be adipocyte, fibroblasts or muscle cells. In particular embodiments, the cells are 3T3-L1 or CHO cells.

In yet another aspect, the invention is directed to a alternative method for identifying a protein involved in insulin stimulated GLUT4 trafficking at the plasma membrane. The method includes introducing an expression library comprising DNA encoding a protein involved in GLUT4 trafficking at the plasma membrane into a population of cells, maintaining the population of cells under conditions suitable for replication of the DNA encoding a protein involved in GLUT4 trafficking at the plasma membrane and for isolating individual clones, subdividing the population of cells into pools of cells such that each pool is a subset of the population of cells, isolating the replicated DNA encoding a protein involved in GLUT4 trafficking at the plasma membrane from the pools of cells, introducing the replicated DNA encoding a protein involved in GLUT4 trafficking at the plasma membrane into cells containing a GLUT4 reporter protein, and assessing trafficking of the reporter protein, wherein altered trafficking of the reporter protein upon insulin stimulation is indicative of the presence of a protein involved in GLUT4 trafficking at the plasma membrane. The method can further comprise additional steps of subdividing the population of cells into pools of cells such that each pool is a subset of the population of cells, isolating the replicated DNA encoding a protein involved in GLUT4 trafficking at the plasma membrane from the pools of cells, introducing the replicated DNA encoding a protein involved in GLUT4 trafficking at the plasma membrane into cells containing a GLUT4 reporter protein, and assessing trafficking of the reporter protein for the pools of cells containing a protein of interest.

Also described herein are proteins identified by the present method, as well as additional proteins identified as containing significant homology to those proteins. In one embodiment, the method of the present invention, along with additional homology studies, has resulted in identification and isolation of proteins that are a family of UBX-domain containing proteins that have been shown to be conserved in mice and humans. These proteins are referred to herein as “UBX-domain proteins” and include the proteins identified herein as mmL1 (SEQ ID NO.: 1), mmL2 (SEQ ID NO.: 2), hsL1 (SEQ ID NO.: 3) and hsL2 (SEQ ID NO.: 4), including both the long and short splice variant forms of the L1 proteins. In a particular embodiment, the protein is a full-length protein which comprises a UBX domain. In alternative embodiments, the protein is a portion of a full-length protein which comprises a UBX domain. In particular embodiments, the protein is one of SEQ ID NO: 1, SEQ ID NO: 2, SEQ ID NO: 3 or SEQ ID NO: 4.

In embodiments, the invention is directed to an isolated antibody or antigen binding fragment thereof which specifically binds to SEQ ID NO: 1, SEQ ID NO: 2, SEQ ID NO: 3 or SEQ ID NO: 4. Such an antibody can be a monoclonal antibody, or alternatively such an antibody can be a polyclonal antibody.

In another aspect, the invention is directed to a method of identifying an agent which alters insulin stimulated GLUT4 trafficking at the plasma membrane. The method comprises maintaining test cells in which GLUT4 is transferred to the cell surface upon stimulation with insulin in the presence of an agent which binds to a UBX domain (or a protein containing a UBX-domain), measuring the proportion of GLUT4 at the cell surface of the test cells after insulin stimulation, and comparing the proportion of GLUT4 at the cell surface in suitable control cells after insulin stimulation, wherein an altered proportion of GLUT4 at the cell surface in the test cells compared to the proportion of GLUT4 in the control cells is indicative of altered insulin stimulated GLUT4 trafficking at the plasma membrane. In embodiments, GLUT4 trafficking is increased in the presence of the agent, or alternatively, is decreased in the presence of the agent. The agent can bind to a protein comprising the sequence of SEQ ID NO: 1, SEQ ID NO: 2, SEQ ID NO: 3 or SEQ ID NO: 4. In a particular embodiment, the agent binds to L1.

In another aspect, the invention is directed to a method for identifying an agent which binds to a protein comprising a UBX domain comprising the steps of isolating the protein, contacting the agent with the isolated protein under conditions suitable for binding of the agent to the isolated protein and detecting a resulting agent-protein complex. In particular embodiments, the agent binds to the proteins of SEQ ID NO: 1, SEQ ID NO: 2, SEQ ID NO: 3 or SEQ ID NO: 4.

In a further aspect, the invention is directed to a method of identifying an agent which alters the interaction between GLUT4 and L1 comprising combining GLUT4, L1 and an agent under conditions appropriate for interaction between GLUT4 and L1, determining the extent to which GLUT4 and L1 interact, and comparing the extent of the GLUT4-L1 interaction in the presence of the agent with the extent of GLUT4-L1 interaction in the absence of the agent, whereby if the extent of the GLUT4-L1 interaction differs significantly in the presence of the agent when compared to the interaction in the absence of the agent, then the candidate agent is one which alters interaction between GLUT4 and L1. In embodiments, the agent enhances the interaction, or alternatively, the agent inhibits the interaction.

In another aspect, the invention is directed to a method of altering insulin stimulated GLUT4 trafficking by contacting an insulin responsive cell with rapamycin.

In embodiments, the invention is directed to a method of altering insulin stimulated GLUT4 trafficking comprising contacting a cell with an agent which binds to the UBX domain of a protein selected from the selected from the group consisting of SEQ ID NO: 1, SEQ ID NO: 2, SEQ ID NO: 3 and SEQ ID NO: 4.

A method of inhibiting GLUT4 externalization comprising contacting a GLUT4 L1 complex with an agent that inhibits disassociation of the complex, and a method of enhancing GLUT4 externalization comprising contacting a GLUT4 L1 complex with an agent that enhances disassociation of the complex are further contemplated.

### BRIEF DESCRIPTION OF THE DRAWINGS

The foregoing and other objects, features and advantages of the invention will be apparent from the following more particular description of various embodiments of the invention, as illustrated in the accompanying drawings.

The file of this patent contains at least one drawing in color. Copies of this patent with color drawings will be provided by the Patent and Trademark Office upon request and payment of the necessary fee.

FIG. 1 is a schematic representation of an enrichment strategy to expression clone proteins involved in GLUT4 trafficking at the plasma membrane.

FIG. 2 is a graphic representation of cell surface/total GLUT4 reporter in the presence and absence of insulin stimulation in control, L clones and H1, H2 and H3 clones.

FIG. 3 is a schematic representation of the domain structure of two splice variants, a 51 kD and a 60 kD isoform, of a UBX-containing protein that modulates GLUT4 trafficking. Synapsin is a phosphoprotein found on synaptic vesicles, Coiled-coil may mediate oligomerization and UBS is a domain of unknown function, with a tertiary structure similar to that of ubiquitin which is present in p47, a cofactor for p97-mediated v-SNARE recycling.

FIG. 4 is a graphic representation of cell surface/total GLUT4 over time after insulin addition as indicated which shows that the truncated, UBX-domain containing cDNA likely acts in a “dominant-negative” manner to reduce insulin-stimulated GLUT4 mobilization.

FIG. 5 is a schematic representation of a hypothesis which predicts the binding of a FAF1 UBX-domain to mUbc9 and a L1 UBX-domain to mUbc9.

FIG. 6A-6B are immunoblots which show the results of the assessment of UBX-domain protein expression in a variety of human and murine tissues. Human ASPL is expressed most highly in skeletal muscle (Ladanyi et. al.), while murine L1 is expressed least highly in skeletal muscle.

FIG. 7 shows the results of flow cytometry which demonstrates that expression of the long or short splice variants of L1 causes intracellular retention of the GLUT4 reporter in 293T cells.

FIGS. 8A-8G illustrate the amino acid sequences of several UBX-domain proteins. FIG. 8A shows murine L1 long splice variant (mmL1) (SEQ ID NO: 1). FIG. 8B shows murine L1 short splice variant (mmL1) (SEQ ID NO: 2). FIG. 8C shows human L1 long splice variant (hsL1) (SEQ ID NO: 3). FIG. 8D shows human L1 short splice variant (hsL1) (SEQ ID NO: 4). FIG. 8E shows murine L2 (mmL2) (SEQ ID NO: 5). FIG. 8F shows human L2 (hsL2) SEQ ID NO: 6), and FIG. 8G shows a human L1 partial sequence (hsL1) (SEQ ID NO: 7).

FIG. 9A illustrates the DNA, (SEQ ID NO: 8), and predicted amino acid sequence of the short splice variant of the murine L1 protein (mmL1-short) (SEQ ID NO: 2).

FIG. 9B illustrates DNA, (SEQ ID NO: 9), and predicted amino acid sequence of the long splice variant of the murine L1 protein (mmL1-long) (SEQ ID NO: 1).

FIG. 10 compares the amino acid sequence of the long splice variant of the human L1 protein (hsL1-long), (SEQ ID NO: 3), and the long splice variant of the murine L1 protein (mmL1-long), (SEQ ID NO: 1), showing the consensus sequence of the two, (SEQ ID NO: 10).

FIG. 11 is a graphic representation of the fraction of GLUT4 contained in various cellular locations over time after addition of insulin. Modeling of the data suggests that GLUT4 derived from insulin-responsive vesicles, not endosomes, is the major contributor to increase at the plasma membrane immediately after insulin stimulation.

FIG. 12 is an immunoblot illustrating that insulin stimulates translocation of the GLUT4 reporter in 3T3-L1 preadipocytes.

FIG. 13 is an immunoblot illustrating that insulin mobilizes endogenous GLUT4 in 3T3-L1 adipocytes with biphasic kinetics.

FIG. 14 is a drawing representing the structure of the FAF1 UBX domain.

FIG. 15 is an immunoblot illustrating that most membrane-associated L1 is present in low density microsomes.

FIG. 16 is an immunoblot illustrating that L1 is not translocated with GLUT4 out of low density microsomes upon insulin stimulation of 3T3-L1 cells.

FIGS. 17A-17F illustrate various aspects of an assay for changes in the proportion of GLUT4 at the plasma membrane.

FIG. 17A is a schematic representation of a modified GLUT4 reporter containing myc epitope tags in the first exofacial loop and green fluorescent protein (GFP) in frame at the carboxy terminus.

FIG. 17B shows the results of time-lapse video fluorescence microscopy of a 3T3-L1 adipocyte expressing the reporter which demonstrates GFP fluorescence in the perinuclear location characteristic of GLUT4.

FIG. 17C shows the results of the use of flow cytometry to quantitate the insulin-stimulated change in the proportion of GLUT4 at the plasma membrane of 3T3-L1 adipocytes expressing the reporter protein.

FIG. 17D shows low density microsomal (LDM) fractions from unstimulated 3T3-L1 adipocytes expressing the reporter, or from control cells not expressing the reporter used in vesicle immunopurification experiments. The lower panels present a similar immunoblot of the supernatants and demonstrate that even though the immunopurification did not quantitatively remove all of the GLUT4 reporter, the endogenous GLUT4 is depleted as expected.

FIG. 17E shows the results of flow cytometry used to quantify the insulinstimulated change in the proportion of GLUT4 as the plasma membrane of 3T3-L1 adipocytes expressing the reporter protein. Phencoerythrin (PE) and Green Fluorescent Protein (GFP) fluorescence intensities are plotted on the vertical and horizontal axes of the dotplots presented.

FIG. 17F shows the results of flow cytometry used to measure insulin-stimulated GLUT4 translocation in confluent Chinese Hamster Ovary (CHO) cells. PE fluorescence (proportional to cell surface GLUT4 reporter) is plotted on the vertical axis and GFP fluorescence (proportional to total GLUT4 reporter) is plotted on the horizontal axis; both axes are logarithmic. Background (unstained) cells expressing the reporter are shown in blue, basal and insulin-stimulated populations are shown in red and green, respectively.

FIGS. 18A-18C show adipose differentiation and GLUT4 translocation in 3T3-L1 cells.

FIG. 18A shows phase contrast (upper left) and bright field (upper right and lower left and right) microscopy of cells at the indicated days of differentiation (scale bar, 50 μM).

FIG. 18B shows confluent 3T3-L1 preadipocytes (“Day 0”) or 3T3-L1 cells that had undergone differentiation for various lengths of time stimulated or not with insulin (160 nM, 10 min.)

FIG. 18C illustrates a comparison of samples treated with either 100 nM wortmannin (Jiang, B. H., et al. 1998 J Biol Chem 273:11017-24) or 50 μM LY294002 (Bradley, R. L. and Cheatham, B. 1999 Diabetes 48:272-8; Cheatham, B., et al., 1994 Mol Cell Biol 14:4902-1113) for 40 minutes prior to insulin addition, as noted. The numbering on the vertical scale indicates a relative measure of GLUT4 at the cell surface, and these arbitrary units cannot be compared in absolute terms to those in other figures.

FIGS. 19A-19C illustrate the mechanics of GLUT4 trafficking in 3T3-L1 cells.

FIG. 19A shows confluent 3T3-L1 preadipocytes (“Day 0”) or 3T3-L1 cells at various stages of adipocyte differentiation (as indicated) treated with insulin for various lengths of time, and notes changes in the proportion of GLUT4 reporter present at the cell surface. Data are plotted for basal cells and for cells treated with 80 nM insulin for 0.5, 1, 1.5, 2, 3, 4, 5, 6, 8, 10, 12, 15, 20, or 30 minutes. The numbering on the vertical scale indicates a relative measure of GLUT4 at the cell surface, and these arbitrary units cannot be compared in absolute terms to those in other figures.

FIG. 19B illustrates insulin stimulated translocation of endogenous GLUT4 to the plasma membrane of 3T3-L1 adipocytes analyzed by subcellular fractionation.

FIG. 19C shows 3T3-L1 preadipocytes (“Day 0”) or cells at various times during adipocyte differentiation stimulated with 80 nM insulin for 20 minutes.

FIG. 20 is a graphic representation of the kinetics of GLUT4 trafficking in 3T3-L1 preadipocytes and NIH 3T3 cells.

FIG. 21 comprises photographs illustrating that culture conditions modulate the kinetics of insulin-stimulated GLUT4 translocation in CHO cells.

FIGS. 22A-22B are graphic representations illustrating that amino acid concentrations regulate the amount of rapidly insulin-mobilized GLUT4 in CHO cells.

FIG. 22A shows CHO cells expressing the reporter cultured in the indicated media for 36 hours, and serum-starved during the last 12 hours of this period.

FIG. 22B illustrates the results of an experiment performed with cells cultured in minimum essential medium (MEM) containing various concentrations of essential amino acids.

FIG. 23 contains a series of graphs illustrating that rapamycin treatment diminishes the amount of rapidly insulin-mobilized GLUT4 in CHO cells.

FIG. 24 is a graph demonstrating that amino acid sufficiency modulates insulin-stimulated GLUT4 translocation in 3T3-L1 adipocytes.

FIG. 25 is a graph demonstrating that treatment with rapamycin diminishes insulin-stimulated GLUT4 translocation in 3T3-L1 adipocytes.

FIG. 26 is a schematic representation of the probable functional domains of a L1 long splice variant.

FIG. 27 is an immunoblot illustrating that two splice variants of the L1 protein are differentially expressed during 3T3-L1 adipocyte differentiation.

FIG. 28 is an immunoblot illustrating that L1 is present on membranes and in cytosol of 3T3-L1 adipocytes and may be cleaved to generate a C-terminal 42 kD fragment.

FIG. 29 is an immunoblot illustrating that the carboxy terminal fragment of L1 is present in Triton-insoluble buoyant fraction and in the Triton-soluble pellet.

FIG. 30 is an illustration representing a further hypothesis proposing that cleavage of L1 by ubiquitin hydrolase-like enzyme plays a role in untethering GLUT4 and allowing it to move to the cell surface and/or away from lipid rafts.

FIG. 31 is an immunoblot illustrating that endogenous L1 associates with the GLUT4 reporter and this complex is disassembled after insulin addition to 3T3-L1 adipocytes.

FIG. 32 is an immunoblot illustrating that L1 and GLUT4 coimmunoprecipitate from lysates of cotransfected 293T cells.

FIG. 33 shows a comparison of the UBX domains of several proteins including FAF1-UBX (SEQ ID NO: 17), L1UB3 (SEQ ID NO: 18), L1UB2 (SEQ ID NO: 19), Sumo (SEQ ID NO: 20), Ubiquitin (SEQ ID NO: 21) and L1UB1 (SEQ ID NO: 22), as well as a consensus sequence of the domains in the various proteins (SEQ ID NO: 23).

FIG. 34 illustrates that the biphasic kinetics of the translocation of GLUT4 in particular cells can be calculated using differential equations to represent the different pathways utilized. (Yeh et al., Biochemistry (1955) 34:15523-31.)

FIG. 35 shows the results of a study to predict the secondary structure of the L1 protein.

FIG. 36 is a schematic illustrating regions of L1 required to sequester GLUT4 intracellularly in cotransfected 293T cells.

### DETAILED DESCRIPTION OF THE INVENTION

Described herein is an expression cloning method useful for identification and isolation of proteins involved in insulin stimulated GLUT4 trafficking. In one embodiment, the expression cloning method is an enrichment method to expression clone proteins involved in GLUT4 trafficking at the plasma membrane of cells, e.g., insulin-responsive cells. In this embodiment, the assay used to identify cells expressing proteins involved in insulin stimulated GLUT4 trafficking is described in detail in U.S. Provisional Applications 60/138,237 filed Jun. 9, 1999, entitled “Method of Measuring Plasma Membrane Targeting of GLUT4 and Expression Cloning of Proteins Involved in GLUT4 Trafficking” and 60/154,078 filed Sep. 15, 1999, entitled “Method of Measuring Plasma Membrane Targeting of GLUT4 ” and U.S. Utility application Ser. No. 09/591,025 filed Jun. 9, 2000, entitled “Method of Measuring Plasma Membrane Targeting of GLUT4” can be used. The entire teachings of the three referenced applications are incorporated herein by reference.

In the method of enriching an expression library for proteins likely to be involved in insulin stimulated GLUT4 trafficking at the plasma membrane, cells can be sorted by the assay method referenced above and the sorted cells cultured, i.e., expanded, using methods well known to those of skill in the relevant art.

The method can be carried out in any type of eukaryotic cell, e.g., manmmalian cells, including human, hamster, mouse and rat, avian or reptilian cells. In embodiments, eukaryotic cells in which GLUT4 trafficking stimulated by insulin takes place are utilized. As used herein, the term “GLUT4 trafficking” encompasses the translocation of GLUT4 from the interior of the cell to the plasma membrane as well as the translocation of GLUT4 from the plasma membrane to the interior of the cell. Cells can be differentiated or undifferentiated and in particular embodiments are adipocytes, fibroblasts or muscle cells, e.g., skeletal muscle cells. Cells can be 3T3-L1 cells, differentiated or undifferentiated, or Chinese Hamster Ovary (CHO) cells.

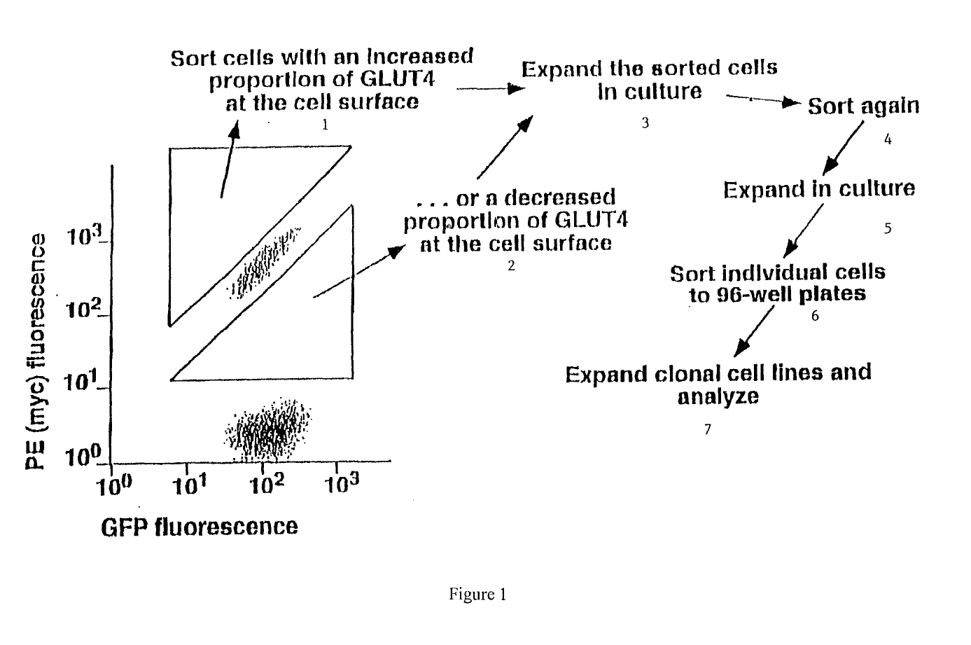

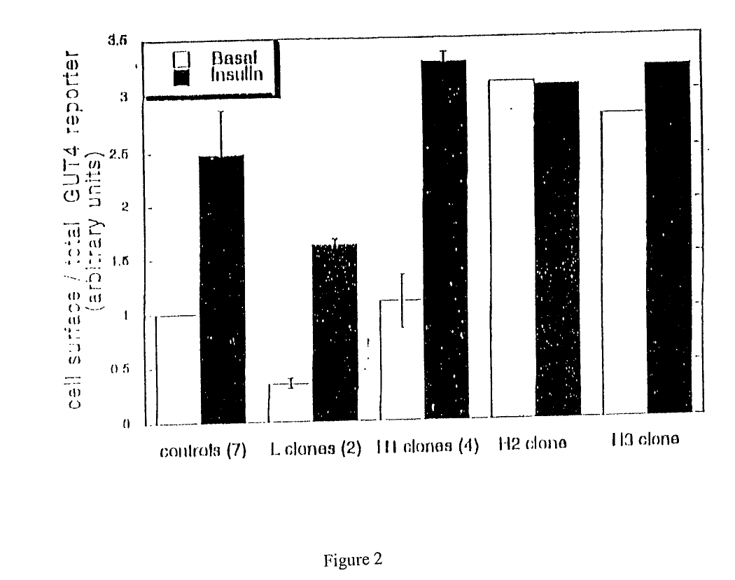

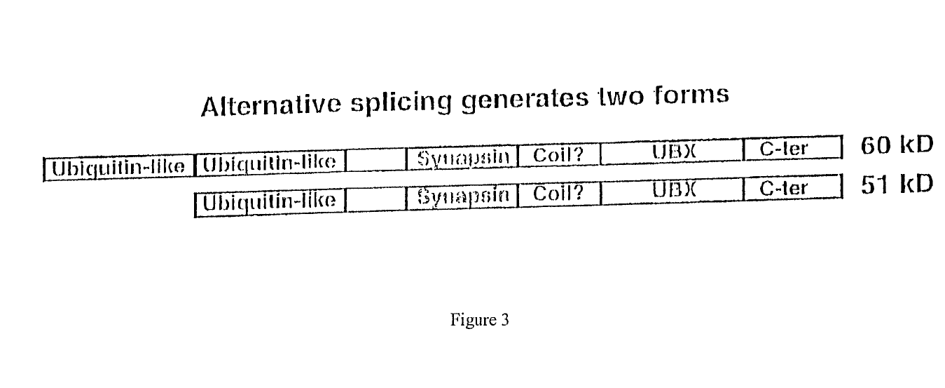

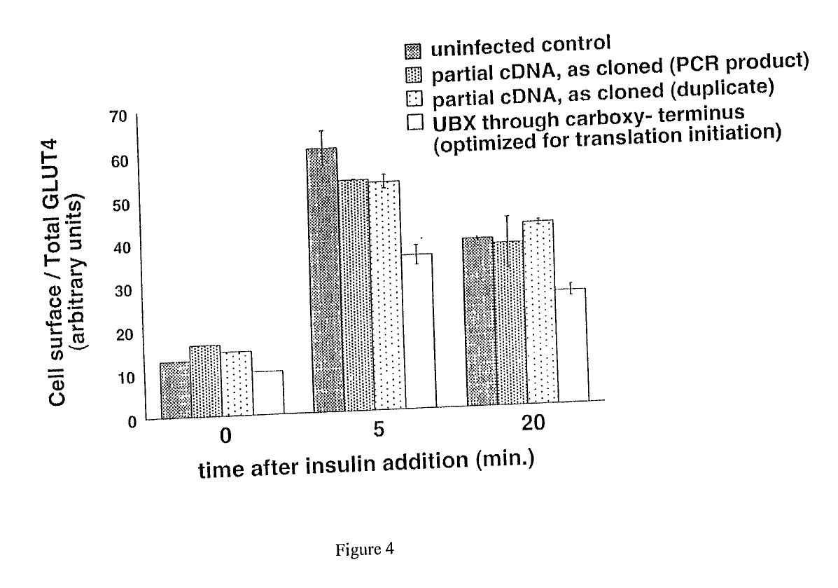

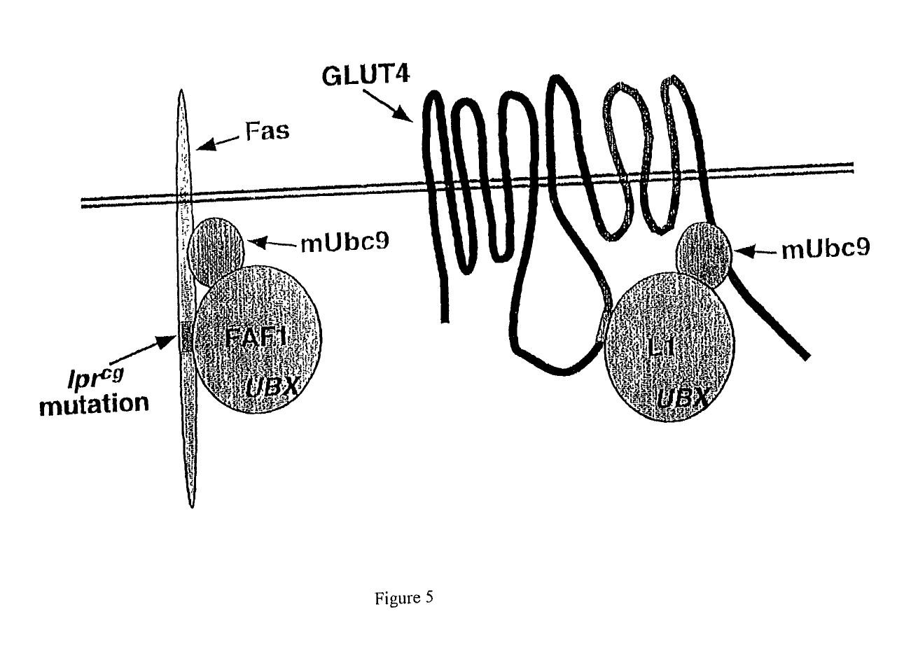

In one embodiment, illustrated in FIG. 1, an expression library is sorted for those cells which exhibit an altered proportion of GLUT4 at the cell surface, an increased proportion 1 or a decreased proportion 2. The sorted cells are expanded in culture 3, then sorted 4 and expanded again 5. The term “sorted” as used herein refers to the process of identifying cells which as the result of any treatment or any altered condition, e.g., transfection, stimulation, e.g., stimulation with insulin, exhibit a proportion of GLUT4 at the plasma membrane which is different from the proportion of GLUT4 at the plasma membrane prior to the treatment or altered condition, and separating those cells from cells not exhibiting like or similar differences. Cells can be separated based on any desired criteria, but are typically separated such that cells, groups of cells, or pools of cells which exhibit similar changes in the proportion of GLUT4 at the plasma membrane after treatment or altered condition are formed. In particular embodiments, cells are sorted to identify and separate cells which exhibit the greatest change, e.g., the greatest increase or decrease, in the proportion of GLUT4 at the plasma membrane. Groups of cells which have been separated based on their similar response to insulin stimulation can be said to have been “enriched” for expression products related to GLUT4 trafficking because such groups, on average, will contain more of such products than will unsorted cells. After expansion by any method known in the art, the expanded cells are sorted again, thus further enriching the average contents of the groups of cells for expression products related to GLUT4 trafficking. Any number of such sortings and expansions can take place, for example, 2, 3, 4, 5, 6, 7, 8, 9, 10 or more, but typically, the final sorting results in individual cells being expanded, e.g., in wells of a 96-well plate 6. Clonal cell lines are then expanded and analyzed 7.

It has been determined that certain cells, for example, CHO cells, when cultured in media with a high amino acid content, such as Dulbecco's modified Eagle's medium (DMEM), respond to insulin stimulation with GLUT4 trafficking which exhibits a biphasic pattern, a first peak which then decreases to a final steady-state proportion of GLUT4 at the plasma membrane. While not wishing to be bound by theory, one possible explanation for this biphasic pattern is that the first “overshoot” type peak is indicative of a bolus of GLUT4 released from the insulin sensitive compartment in which it has been sequestered, and that the resulting steady-state which follows is indicative of a continued release of GLUT4 through another pathway after the GLUT4 in the insulin sensitive compartment has been depleted. This biphasic pattern can be advantageously exploited, e.g., to identify cells most likely to contain proteins involved in insulin stimulated GLUT4 trafficking. The proportion of GLUT4 at the plasma membrane in cells can be measured during either phase, but typically is measured towards the end of the first or “overshoot” type peak phase. Measurements can be taken at 1, 2, 3, 4, 5, 6 or 7 minutes after insulin stimulation to identify those cells most likely to contain proteins involved in GLUT4 translocation. In one embodiment, the time at which such measurement is taken is about 5 minutes after insulin stimulation.

Therefore, clonal cell lines can be expanded in media with high amino acid content. The phrase “high amino acid content” as used herein is intended to encompass media with an amino acid content greater than that contained by F12 media. Such media can be commercially obtained, e.g., Dulbecco's modified Eagle's medium (DMEM), or it can be prepared by adding amino acids to standard media.

In addition to the enrichment strategy described in the preceding paragraphs, pools of clones that exhibit insulin stimulated GLUT4 trafficking can be subjected to sib-selection and further analysis until a single cDNA encoding a protein involved in the trafficking is obtained. The term “sib-selection” as used herein refers to a system of dividing and sub-dividing a large cDNA library into a manageable number of pools, each pool consisting of between about 2 to about 1000 clones. These pools are then tested for the protein of interest. After a pool containing the protein of interest is identified, the pool is subdivided into successively smaller pools, each of which is retested until the single cDNA of interest is isolated. By assigning individual clones to sub-pools in a matrix format, sib-selection and analysis can be performed more rapidly.

Although insulin stimulated GLUT4 trafficking can be measured by any method known in the art to enrich an expression library, in one method, insulin stimulated GLUT4 trafficking is assessed as described in U.S. Pat. No. 6,303,373, the teachings of which have been previously incorporated by reference, using a modified GLUT4 protein. Modified GLUT4 is GLUT4 protein that includes one or more detectable tags, such as an epitope or other label, in an extracellular domain and a detectable tag or tags, such as a fluorescent tag, e.g., a fluorescent protein, in an intracellular domain. The first epitope tag can be from any protein other than GLUT4, or can be from GLUT4 itself if it can be detected specifically when outside of a cell, provided that whatever epitope tag is used, it does not interfere with translocation of the modified GLUT4 protein. The epitope tag need not be detected using an antibody. It can, for instance, have enzymatic activity that allows detection only when it is extracellular. The second detectable tag corresponds to total cellular GLUT4. Typically, the tag is in an intracellular region of the modified GLUT4 protein, but it can be present in an extracellular region, provided that its detectable characteristic, e.g., fluorescence, is not altered by changes in conditions, e.g., pH, ionic concentrations, which occur when the modified protein, Red Fluorescent Protein (RFP) or Blue Fluorescent Protein (BFP), corresponds to total GLUT4 in the cell and does not change in quantity moves to the cell surface. The second detectable tag, e.g., the fluorophore, such as Green Fluorescent Protein (GFP), or RFP or BFP corresponds to total GLUT4 in the cell and does not change in quantity depending on the location of the protein within the cell. In contrast, the first epitope tag causes fluorescence only if it is extracellular, since only then is it recognized by an antibody, that cannot cross the cell membrane and, thus, can recognize only extracellular epitopes. In other words, the intracellular tag is always detectable, but the extracellular tag is only detectable if GLUT4 is translocated. The antibody that recognizes the epitope can itself be detectably, e.g., fluorescently, labeled or can, in turn, be recognized by a secondary antibody that carries a detectable label, e.g., a fluorophore. In those instances in which modified GLUT4 is detected in an assay by means of fluorescence, the two fluorescent labels (one to detect the epitopes in the first extracellular domain and one present in an intracellular domain) used must be different (detectable at different wavelengths). For example, if a fluorescently-labeled antibody is used to detect the epitope tag of modified GLUT4, the fluorescent moiety on the antibody must be detectable at a wavelength different from the wavelength at which the fluorescent tag, e.g., GFP or BFP, in the intracellular domain of GLUT4 is detected.

In another aspect, the invention is directed to the expression library formed by the method. Such expression libraries are enriched in expression products encoding proteins involved in GLUT4 trafficking at the plasma membrane. In particular embodiments, the expression libraries contain nucleic acids, e.g., cDNAs, encoding proteins comprising UBX domains. After an enriched expression library is obtained, the cells of the library can be screened to identify cells containing nucleic acids of interest, i.e., those encoding proteins involved in insulin stimulated GLUT4 trafficking. Such nucleic acids, for example, can encode a UBX domain protein or a portion or fragment thereof, e.g., mmL1 (SEQ ID NO: 1 and SEQ ID NO: 2) and hsL1 (SEQ ID NO: 3 and SEQ ID NO: 4) referred to as “L1 proteins” and mmL2 (SEQ ID NO: 5), and hsL2 (SEQ ID NO: 6) referred to as “L2” proteins”, including both the long and short splice variants of the L1 proteins.

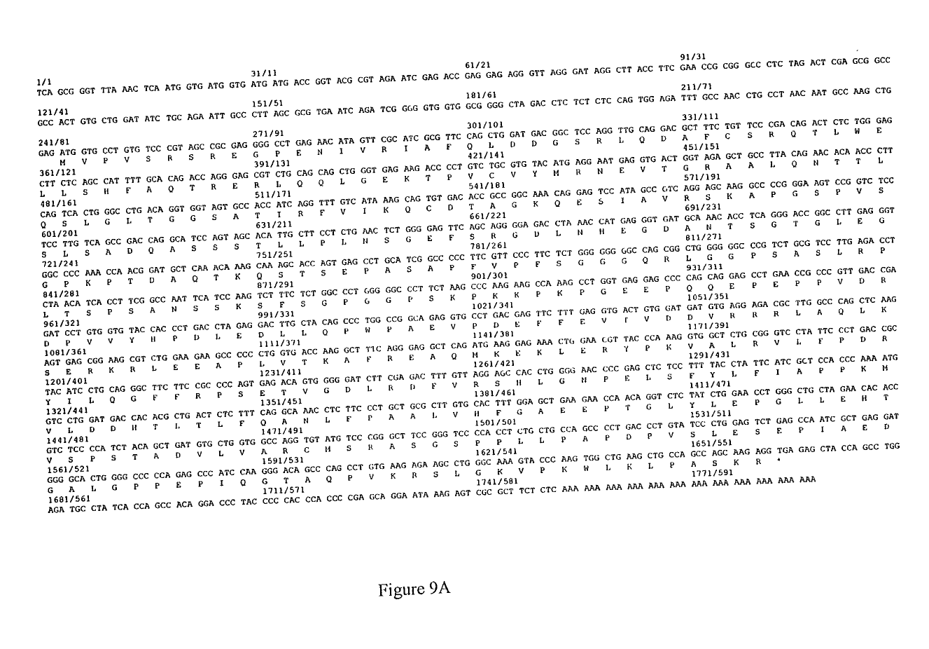

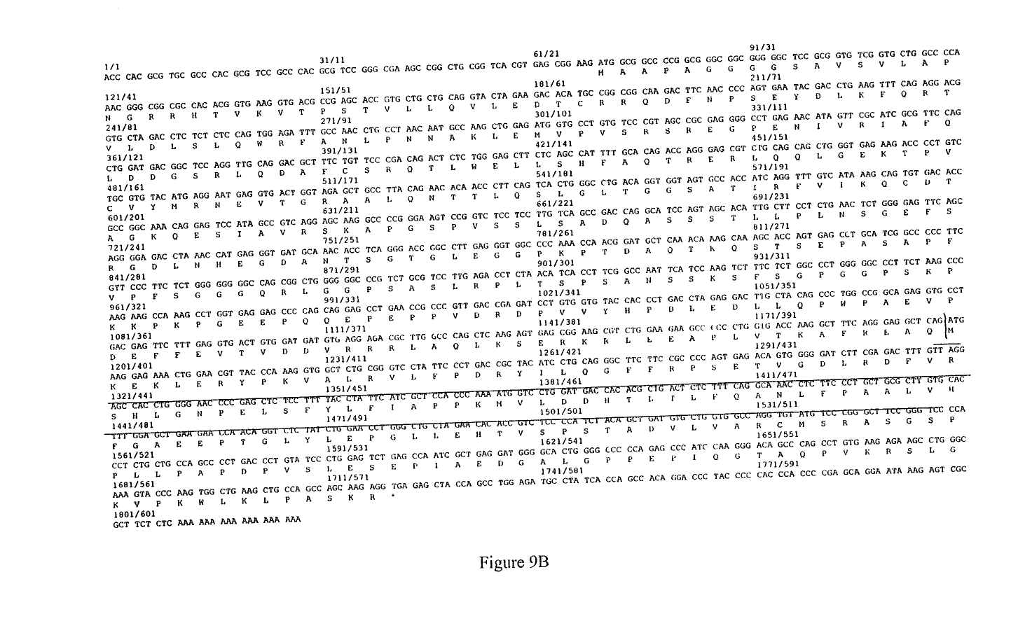

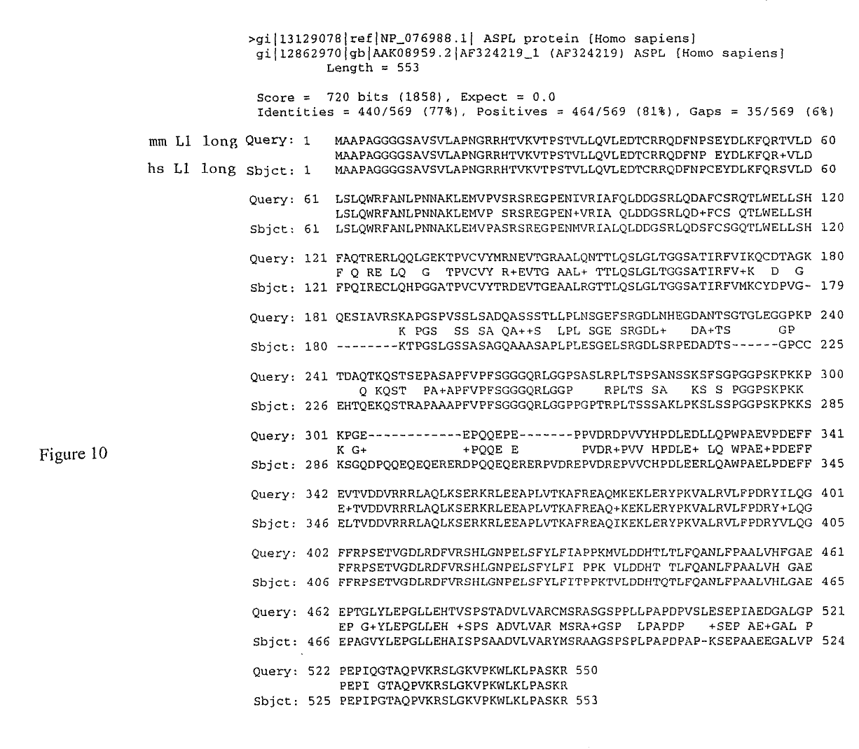

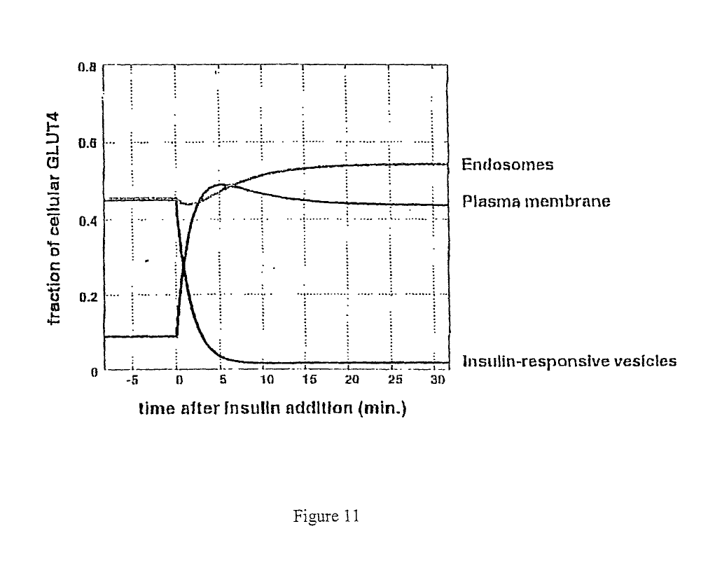

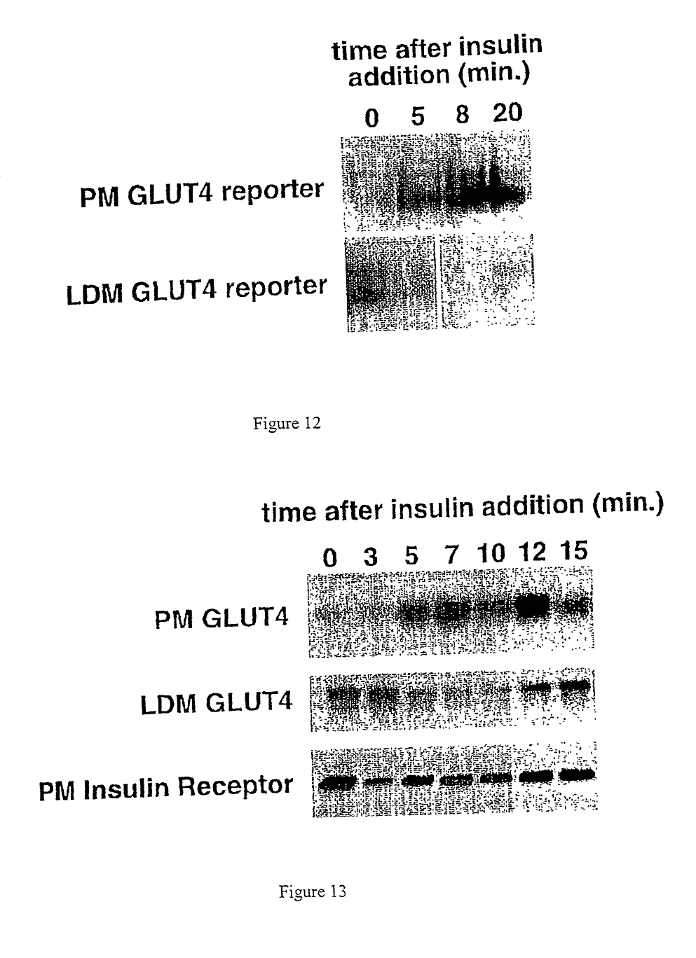

As indicated above, L1 proteins demonstrate two isoforms, a short splice variant (˜51 kD) and a long splice variant (˜60 kD). The DNA and predicted amino acid sequences for alternative splice variants of the murine L1 protein are shown in FIGS. 9A and 9B. The UBX-domain is also illustrated in FIG. 9B. L2 proteins show close homology to L1 proteins and were identified using that property.

Nucleic acids referred to herein as “isolated” are nucleic acids separated away from the nucleic acids of the genomic DNA or cellular RNA of their source of origin, e.g., as it exists in cells or in a mixture of nucleic acids such as a library, and may have undergone further processing. “Isolated” nucleic acids include nucleic acids obtained by methods described herein, similar methods or other suitable methods, including essentially pure nucleic acids, nucleic acids produced by chemical synthesis, by combinations of biological and chemical methods, and recombinant nucleic acids which are isolated.

Any collection of nucleic acid, e.g., genomic DNA fragments or cDNAs, likely to encode a protein involved in insulin stimulated GLUT4 trafficking can be utilized to form the original expression library. The nucleic acids can be prepared using any suitable method. For example, the nucleic acids can be a genomic DNA library prepared by a partial digestion of genomic DNA, e.g., human genomic DNA, with Sau3A I, Mbo I or other suitable restriction enzymes. In one embodiment, the nucleic acids can be a cDNA library. Methods suitable for preparing a cDNA library are well known to those of skill in the art. For example, RNA or preferably polyA⁺ RNA can be isolated from cells by guanidinium isothiocyanate extraction and oligo dT chromatography, or commercially available kits such as RNAgents® Total RNA Isolation System and PolyATtract® mRNA Isolation System (both available from Promega, Madison, Wis.) can be used. cDNAs corresponding to the polyA⁺ RNA can be prepared using a reverse transcriptase for first strand synthesis and a suitable DNA polymerase, e.g., E. coli DNA polymerase I, for second strand synthesis. Commercially available kits for making cDNA, such as Superscript system (Gibco/BRL, Rockville, Md.) can be used.

The cDNAs can be ligated into an expression vector or ligated to DNA linkers, also referred to as adapters, which contain suitable restriction sites to facilitate cloning into a desired expression vector. Linkers which contain a variety of restriction sites are available from commercial sources (e.g., Pharmacia Biotech, Piscataway, N.J.; New England Biolabs, Beverly, Mass.). If desired, the cDNAs can be further processed before ligation into an expression vector. For example, the cDNAs can be size fractionated, e.g., by centrifugation through a sucrose gradient or by electrophoresis through agarose gel, to enrich for full length cDNAs.

The nucleic acids, e.g., cDNA library, can be ligated into a suitable expression vector to produce an expression library. The library can be ligated into the vector such that nucleic acids are inserted into the vector in a preferred orientation. Expression vectors suitable for use in the invention contain sequences which direct expression, transcription and translation, of the insert nucleic acid in a suitable expression system, e.g., in vitro expression, expression in eukaryotic cells. The expression vector can also contain a selectable marker for selection of cells carrying the vector. Many suitable selectable markers are known, for example, genes which confer resistance to antibiotics such as the β-lactamase gene for ampicillin resistance and the Tet gene for tetracycline resistance.

Although retroviral vectors are an effective means of infecting cells with nucleic acids isolated from the expression library, the selection of a suitable expression vector will be dependent on the desired method of expression. For example, for in vitro expression, vectors which contain a promoter for Sp6 or T7 RNA polymerase, such as pSP64 or pGEMEX (Promega, Madison, Wis.) can be used. For expression in mammalian cells, an expression vector which contains a promoter suitable to drive expression of the inserted nucleic acid, e.g., simian virus 40 early or late promoter, Rous sarcoma virus long terminal repeat promoter, cytomegalovirus promoter, adenovirus late promoter can be selected. Suitable expression vectors for expression in mammalian cells include, for example, pCDM8, pCDNA1.1/amp, pcDNA3.1, pRc/RSV, pEF-1 (Invitrogen, Carlsbad, Calif.), pCMV-Script®, pFB, pSG5, pXT1 (Stratagene, La Jolla, Calif.), pCDEF3 (Goldman, L. A., et al., Biotechniques, 21:1013-1015 (1996)), pSVsport (Gibco/BRL, Rockville, Md.), pEF-Bos (Mizushima, S., et al., Nucleic Acids Res., 18:5322 (1990)) and the like.

Host cells into which an expression library has been inserted can be cultured under conditions suitable to produce individual colonies (clones), and one or more pools of individual colonies can be collected. Pools of colonies can be collected using any suitable procedure. In one embodiment, all of the colonies from a plate form a pool. In this situation, the colonies can be collected by adding a quantity of media to the plate which is sufficient to wet the surface, e.g., about 2 mL, and scraping the colonies off of the plate, thereby forming a suspension which can be recovered. If desired, a pool can consist of the colonies from a fraction of a plate or the colonies from two or more plates, and suitable collection procedures can be employed.

The expression library (expression plasmids) contained within the pool of cells can be recovered from the pool immediately following collection, or the pool can be cultured to provide amplification of the number of expression plasmids that can be recovered. The expression library can be recovered using any suitable method for isolating nucleic acids. For example, by using methods for recovering plasmids, such as the alkaline lysis method or using commercially available kits. In one example, the expression library can be recovered using QIAprep spin columns (QIAGEN, Valencia, Calif.).

Expression in eukaryotic cells can be accomplished by inserting or introducing nucleic acids into suitable host cells and culturing the resulting cells under conditions suitable for expression of the library. The expression library can be inserted using any suitable method. The expression library recovered from a pool can be inserted into eukaryotic cells, e.g., yeast, insect cells, mammalian cells, by transformation, transfection, infection or other suitable methods. In one embodiment, the expression library is expressed in a mammalian cell. Mammalian cells can include adipocytes, fibroblasts and muscle cells, e.g., skeletal muscle cells. The cells can be 3T3-L1 or CHO cells, either differentiated or undifferentiated. The expression library can be inserted into the mammalian cell by transfection, for example by the calcium phosphate method, diethylaminoethyl (DEAE) dextran method, electroporation or using liposomes, e.g., LipofectAMINE™, Gibco/BRL. The transfected mammalian cells can be cultured under conditions suitable for expression of the expression constructs. The expressed pool of proteins can be recovered using any suitable methods.

In one embodiment, transfected mammalian cells can be cultured in media supplemented with growth factors and/or a high concentration of serum, e.g., about 20% or more, for a period of about 12 to about 24 hours. The media can then be replaced with serum-free media or with media that is supplemented with a lower concentration of serum, e.g., about 10% or less, and the cells can be cultured for a period of time sufficient for expression, e.g., about 24 to about 72 hours. As previously described, for certain cells, e.g., CHO cells, culturing in media with high amino acid content such as DMEM is typically selected to assure optimal sequestration of GLUT4 in insulin sensitive compartments.

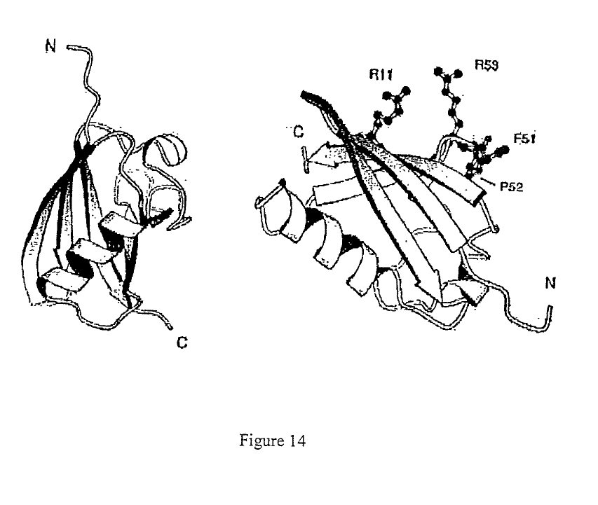

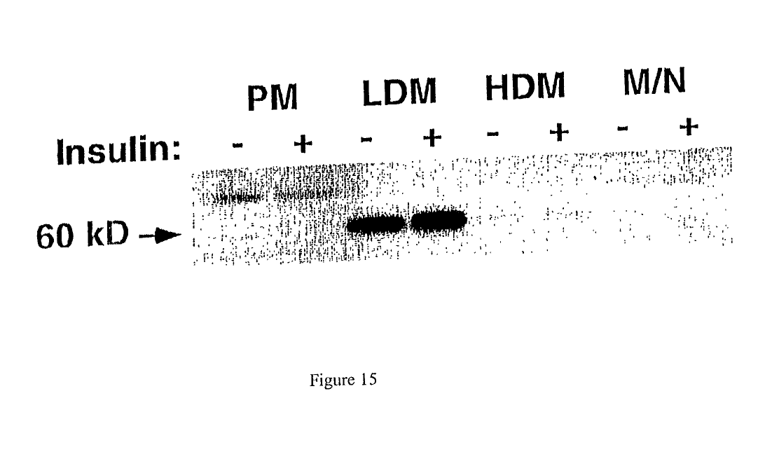

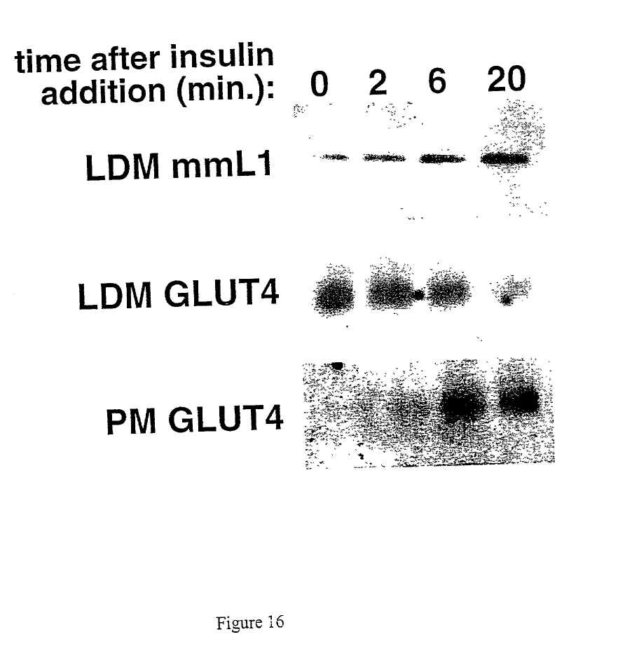

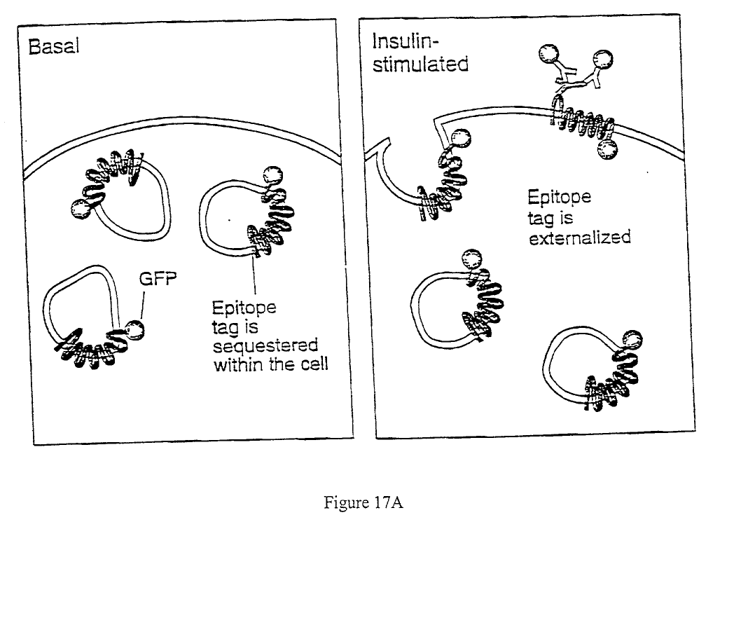

Proteins can be studied in one or more suitable functional assays to determine if a protein of interest has been expressed. For example, certain activities can be determined by assays for binding activity, e.g., binding to GLUT4. Thus, the present invention is useful to identify proteins involved in GLUT4 trafficking. Such proteins can be used to further elucidate the insulin-responsive process in cells and as targets or models for design of drugs that alter (enhance or inhibit) glucose uptake/GLUT4 movement in cells. The method of the present invention has resulted in identification and isolation of proteins that are a family of coiled-coil-UBX-domain containing proteins that have been shown to be conserved in mice and humans. The structure of an FAF1 UBX domain protein is illustrated in FIG. 14 and the sequences, both the encoding nucleic acid sequence and the amino acid sequence, are identified, for example, for a murine UBX domain protein in FIG. 9B.

In a particular embodiment, the protein is a full-length protein which comprises a UBX domain. In alternative embodiments, the protein is a portion of a full-length protein which comprises a UBX domain. As used herein “UBX domain” refers to a domain found in proteins involved in trafficking and “UBX domain proteins” refer to a naturally occurring or endogenous protein with a UBX domain. The terms also encompass recombinant proteins, synthetic proteins, i.e., proteins produced using the methods of synthetic organic chemistry. Accordingly, as defined herein, the term includes mature protein, polymorphic or allelic variants, and other isoforms of a UBX domain protein, e.g., produced by alternative splicing or other cellular processes, and modified or unmodified forms of the foregoing, e.g., lipidated, glycosylated, unglycosylated. Naturally occurring or endogenous UBX domain proteins include wild type proteins such as a mature human UBX domains, polymorphic or allelic variants and other isoforms which occur naturally, e.g., in mammals including humans and nonhuman primates. Such proteins can be recovered or isolated from a source which naturally produces a UBX domain protein, for example.

“Functional variants” of UBX domain proteins include functional fragments, functional mutant proteins, and/or functional fusion proteins which can be produced using suitable methods, e.g., mutagenesis such as chemical mutagenesis and radiation mutagenesis or by recombinant DNA techniques. A “functional variant” is a protein or polypeptide which has at least one function characteristic of a UBX domain protein, such as a binding activity, a signaling activity or the ability to alter GLUT4 trafficking (increase or decrease).

In one embodiment, a functional variant of a UBX domain protein shares at least about 80% amino acid sequence identity with a naturally occurring UBX domain protein, preferably at least about 90% amino acid sequence identity, and more preferably at least about 95% amino acid sequence identity with a naturally occurring UBX domain. Amino acid sequence identity can be determined using a suitable sequence alignment algorithm, such as the Lasergene system (DNASTAR, Inc., Madison, Wis.), using the Clustal method with the PAM 250 residue weight table, a gap penalty of 10, a gap length penalty of 10 and default parameters (pairwise alignment parameters: ktuple=1, gap penalty=3, window=4 and diagonals saved=5). In another embodiment, a functional variant is encoded by a nucleic acid sequence which is different from the naturally-occurring nucleic acid sequence, but which, due to the degeneracy of the genetic code, encodes a UBX domain protein or a portion thereof.

Generally, fragments or portions of UBX domain proteins include those having a deletion, i.e., one or more deletions, of an amino acid, i.e, one or more amino acids, relative to the mature UBX domain protein, such as N-terminal, C-terminal or internal deletions. Fragments or portions in which only contiguous amino acids have been deleted or in which non-contiguous amino acids have been deleted relative to mature UBX domain are also envisioned.

Mutant UBX domain proteins include natural or artificial variants of a naturally occurring UBX domain protein differing by the addition, deletion and/or substitution of one or more contiguous or non-contiguous amino acid residues. Such mutations can occur at one or more sites on a protein, for example a conserved region or nonconserved region, an extracellular region, cytoplasmic region, or transmembrane region.

Particular proteins referred to herein as UBX-domain proteins include the proteins identified herein as mmL1 (SEQ ID NO: 1 and SEQ ID NO: 2) and hsL1 (SEQ ID NO: 3 and SEQ ID NO: 4) referred to as “L1 proteins” and mmL2 (SEQ ID NO: 5), and hsL2 (SEQ ID NO: 6) referred to as “L2 ” proteins”. L1 proteins are of particular interest because studies have shown that L1 associates with GLUT4. It is postulated that the binding of L1 to GLUT4 “tethers” GLUT4 into the insulin sensitive compartment and that the release of GLUT4 from its L1 tether is required for translocation of GLUT4 to the plasma membrane. Cleavage of L1 by an ubiquitin hydrolyse-like enzyme may play a role in untethering GLUT4 and allowing it to move to the cell surface and/or away from the lipid rafts. Two splice variants of the L1 protein have been identified. A shorter splice variant with a length of about 51 kD is expressed early in adipocyte differentiation but not when cells are fully differentiated, while a longer splice variant with a length of about 60 kD is continually expressed. The 60 kD L1 is present on membranes and may be cleaved to generate a C-terminal 42 kD fragment. This fragment is present in the lipid rafts which accumulate transiently after insulin addition. It is also known that the L1/GLUT4 complex is disassembled after insulin addition. Moreover, L1 and GLUT4 coimmunoprecipitate from lysates of certain cotransfected cells.

Thus, the invention is further directed to a method for detecting, a protein of interest, or a portion or fragment thereof, in a sample of cells, e.g., an expression library. The method comprises adding to the sample an agent that specifically binds to the protein, and detecting the agent specifically bound to the protein. Appropriate washing steps can be added to reduce nonspecific binding to the agent. The agent can be, for example, an antibody, a ligand or a substrate mimic. The agent can have incorporated into it, or have bound to it, covalently or by high affinity non-covalent interactions, for instance, a label that facilitates detection of the agent to which it is bound, wherein the label can be, e.g., a phosphorescent label, a fluorescent label, a biotin or avidin label, or a radioactive label. The means of detection of the protein can vary, as appropriate to the agent and label used. For example, for an antibody that binds to the protein, the means of detection may call for binding a second antibody, which has been conjugated to an enzyme, to the antibody which binds the protein of interest, and detecting the presence of the second antibody by means of the enzymatic activity of the conjugated enzyme.

Similar principles can also be applied to a cell lysate or a more purified preparation of proteins from cells that may comprise a protein of interest, for example in the methods of immunoprecipitation, immunoblotting, immunoaffinity methods, that in addition to detection of the particular protein, can also be used in purification steps, and qualitative and quantitative immunoassays. See, for instance, chapters 11 through 14 in Antibodies: A Laboratory Manual, E. Harlow and D. Lane, eds., Cold Spring Harbor Laboratory, 1988.

In addition, isolated protein, an antigenically similar portion thereof, especially a portion that is soluble, can be used in a method to select and identify molecules which bind specifically to the protein. Fusion proteins comprising all of, or a portion of, the protein linked to a second moiety not occurring in the protein of interest as found in nature, can be prepared for use in another embodiment of the method. Suitable fusion proteins for this purpose include those in which the second moiety comprises an affinity ligand, e.g., an enzyme, antigen, epitope. Fusion proteins can be produced by the insertion of a gene encoding the protein of interest or a variant thereof, or a suitable portion of such gene into a suitable expression vector, which encodes an affinity ligand, e.g., pGEX-4T-2 and pET-15b, encoding glutathione S-transferase and His-Tag affinity ligands, respectively. The expression vector can be introduced into a suitable host cell for expression. Host cells are lysed and the lysate, containing fusion protein, can be bound to a suitable affinity matrix by contacting the lysate with an affinity matrix.

The fusion protein can be immobilized on a suitable affinity matrix under conditions sufficient to bind the affinity ligand portion of the fusion protein to the matrix, and is contacted with one or more candidate binding agents, e.g., a mixture of peptides, to be tested, under conditions suitable for binding of the binding agents to a portion of the bound fusion protein, e.g., the UBX domain. Next, the affinity matrix with bound fusion protein can be washed with a suitable wash buffer to remove unbound candidate binding agents and non-specifically bound candidate binding agents. Those agents which remain bound can be released by contacting the affinity matrix with fusion protein bound thereto with a suitable elution buffer. Wash buffer can be formulated to permit binding of the fusion protein to the affinity matrix, without significantly disrupting binding of specifically bound binding agents. In this aspect, elution buffer can be formulated to permit retention of the fusion protein by the affinity matrix, but can be formulated to interfere with binding of the candidate binding agents to the target portion of the fusion protein. For example, a change in the ionic strength or pH of the elution buffer can lead to release of specifically bound agent, or the elution buffer can comprise a release component or components designed to disrupt binding of specifically bound agent to the target portion of the fusion protein.

Immnobilization can be performed prior to, simultaneous with, or after, contacting the fusion protein with candidate binding agent, as appropriate. Various permutations of the method are possible, depending upon factors such as the candidate molecules tested, the affinity matrix-ligand pair selected, and elution buffer formulation. For example, after the wash step, fusion protein with binding agent molecules bound thereto can be eluted from the affinity matrix with a suitable elution buffer, e.g., a matrix elution buffer, such as glutathione for a GST fusion. Where the fusion protein comprises a cleavable linker, such as a thrombin cleavage site, cleavage from the affinity ligand can release a portion of the fusion with the candidate agent bound thereto. Bound agent molecules can then be released from the fusion protein or its cleavage product by an appropriate method, such as extraction.

One or more candidate binding agents can be tested simultaneously. Where a mixture of candidate binding agents is tested, those found to bind by the foregoing processes can be separated (as appropriate) and identified by suitable methods, e.g., PCR, sequencing, chromatography). Large libraries of candidate binding agents, e.g., peptides, RNA oligonucleotides, produced by combinatorial chemical synthesis or by other methods can be tested (see e.g., Ohlmeyer, M. H. J. et al., Proc. Natl. Acad. Sci. USA 90:10922-10926 (1993) and DeWitt, S. H. et al., Proc. NatL. Acad. Sci. USA 90:6909-6913 (1993), relating to tagged compounds; see also Rutter, W. J. et al. U.S. Pat. Nos. 5,010,175; Huebner, V. D. et al., 5,182,366; and Geysen, H. M., 4,833,092). Random sequence RNA libraries (see Ellington, A. D. et al., Nature 346:818-822 (1990); Bock, L. C. et al., Nature 355:584-566 (1992); and Szostak, J. W., Trends in Biochem. Sci. 17:89-93 (March, 1992)) can also be screened according to the present method to select RNA molecules which bind to a target fusion protein. Where binding agents selected from a combinatorial library by the present method carry unique tags, identification of individual biomolecules by chromatographic methods is possible. Where binding agents do not carry tags, chromatographic separation, followed by mass spectrometry to ascertain structure, can be used to identify binding agents selected by the method.

In another aspect, the invention is directed to antibodies to the proteins of interest, e.g., UBX domain proteins, e.g., L1. The term “antibody” as used herein refers to immunoglobulin molecules and immunologically active portions of immunoglobulin molecules, i.e., molecules that contain an antigen binding site that specifically binds an antigen. A molecule that specifically binds to a protein is a molecule that binds to that protein or a fragment thereof, but does not substantially bind other molecules in a sample, e.g., a biological sample, which naturally contains the protein. Examples of immunologically active portions of immunoglobulin molecules include F(ab) and F(ab′)₂ fragments which can be generated by treating the antibody with an enzyme such as pepsin. Both polyclonal and monoclonal antibodies that bind to an identified protein are envisioned. The term “monoclonal antibody” or “monoclonal antibody composition”, as used herein, refers to a population of antibody molecules that contain only one species of an antigen binding site capable of immunoreacting with a particular epitope of a protein of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., a protein or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal, e.g., from the blood, and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature, 256:495-497, the human B cell hybridoma technique (Kozbor et al. (1983) Immunol. Today, 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

In yet another aspect, the invention is directed to a method of identifying an agent which alters GLUT4 trafficking at the plasma membrane. The method includes maintaining test cells in which GLUT4 is transferred to the cell surface upon stimulation with insulin in the presence of the agent which binds to a UBX domain, measuring the proportion of GLUT4 at the cell surface of the test cells after insulin stimulation, comparing the proportion of GLUT4 at the cell surface with the proportion of GLUT4 at the cell surface in suitable control cells after insulin stimulation, wherein an altered proportion of GLUT4 a the cell surface in the test cells compared to the proportion of GLUT4 in the control cells is indicative of altered GLUT4 trafficking at the plasma membrane. In one embodiment, GLUT4 trafficking at the plasma membrane is increased. In an alternative embodiment, GLUT4 trafficking at the plasma membrane is increased. In a particular embodiment, the agent binds to a UBX domain of a protein involved in GLUT4 trafficking. In one embodiment, the agent binds to a protein comprising a sequence selected from the group of SEQ ID NO: 1, SEQ ID NO: 2, SEQ ID NO: 3 and SEQ ID NO: 4.

Agents can be any chemical, such as an element, a molecule or a compound. Agents can be produced synthetically, made by recombinant techniques or isolated from natural sources. Candidate agents can be peptides, polypeptides, peptoids, sugars or hormones. Additionally, nucleic acid molecules, e.g., antisense nucleic acid molecules can be utilized as agents. Small molecules or larger molecules produced, e.g., by combinatorial chemistry can be utilized directly or can be compiled into libraries. Such libraries can contain alcohols, alkyl halides, amines, amides, esters, aldehydes, ethers and other organic compounds. Naturally occurring or genetically engineered products isolated from bacterial, animal or plant cells can also be utilized, as can corresponding cell lysates. The agents can be presented for testing in an isolated form or alternatively, as mixtures of compounds.

In yet another aspect, the invention is directed to a method for identifying an agent which alters, e.g., inhibits or enhances, interaction between GLUT4 and a protein of interest, e.g., L1. The term “interaction” as used herein describes the composite of the various forces that govern the connection between two molecules. The term is particularly utilized when describing the connection between two proteins, although its use is not limited to that particular connection. The agent can be, for instance, a substrate, or a substrate mimic, an antibody, or a compound, such as a peptide, that binds with specificity to a site on the protein. The method comprises combining, not limited to a particular order, GLUT4, the protein of interest, e.g., a UBX domain protein, e.g., L1, and a candidate agent to be assessed for its ability to alter, e.g., enhance or inhibit, the interaction, e.g., binding, between GLUT4 and L1, under conditions appropriate for interaction between the GLUT4 and L1, e.g., pH, salt, temperature conditions conducive to appropriate conformation and molecular interactions; determining the extent to which the GLUT4 and L1 interact; and comparing the extent of the GLUT4-L1 interaction in the presence of a candidate agent with the extent of GLUT4-L1 interaction in the absence of a candidate agent. If the extent of the GLUT4-L1 interaction differs, e.g., is enhanced or inhibited in the presence of the agent when compared to the interaction in the absence of the agent, then the candidate agent is one which alters, e.g., enhances or inhibits, interaction between GLUT4 and L1.

The present work initially studied the cell-type specificity of insulin-regulated GLUT4 trafficking. Using a novel reporter molecule to obtain detailed kinetic date, it was found that GLUT4 participates in a highly insulin-responsive compartment not only in the fully-differentiated 3T3-L1 adipocytes employed, but in undifferentiated 3T3-L1 preadipocytes as well. Such a compartment is not present in all cell types, since NIH 3T3 cells do not exhibit highly insulin-responsive trafficking. In CHO cells, highly insulin-responsive trafficking was observed only when the cells were cultured identically to 3T3-L1 adipocytes, in DMEM. In standard F12 culture medium the cells were less responsive. The highly insulin-responsive kinetics correlated with basal state redistribution of intracellular GLUT4 from the perinuclear region into punctate, peripheral structures. The amino acid content of these media were pinpointed as the relevant difference causing these trafficking characteristics: thus, given sufficient concentrations of essential amino acids, GLUT4 accumulates in a highly insulin responsive compartment in CHO cells. The data show that amino acids regulate GLUT4 accumulation in this compartment through a rapamycin-sensitive pathway. Finally, it was also demonstrated that amino acid sufficiency modulates highly insulin-responsive GLUT4 trafficking in 3T3-L1 adipocytes, and that this response is also rapamycin-sensitive. The data are consistent with the concept that both 3T3-L1 cells and CHO cells contain peripheral, highly insulin-responsive compartments through which GLUT4 traffics, and that amino acid sufficiency modulates GLUT4 trafficking through these compartments in both cell types.

The experimental data provided herein demonstrates that insulin triggers rapid exocytosis of GLUT4 in 3T3-L1 adipocytes and preadiopcytes and in CHO cells, but not in NIH 3T3 cells. This conclusion is based on studies using a novel, FACS-based assay to measure changes in the proportion of GLUT4 present at the plasma membrane, and is supported by subcellular fractionation data. This action of insulin is blocked by either of two structurally dissimilar phosphatidylinositol-3-kinase inhibitors in 3T3-L1 cells at all times during differentiation, suggesting that identical signaling mechanisms are involved. Moreover, insulin stimulates GLUT4 externalization with identical, biphasic kinetics at all times during 3T3-L1 differentiation, and the reinternalization and recycling of the reporter protein was demonstrated as well. Strikingly, it was found that when CHO cells are cultured identically to 3T3-L1 adipocytes (in DMEM), the kinetics and initial magnitude of insulin-stimulated GLUT4 redistribution are similar in both cell types. In contrast, when CHO cells are cultured in their usual media (F12), GLUT4 is only minimally distributed to a highly insulin-responsive compartment, assessed kinetically. The presence of GLUT4 in this rapidly mobilized compartment correlates with a basal state redistribution of GLUT4 out of the perinuclear region and into punctate structures in the periphery. The data also demonstrates that the difference in GLUT4 targeting in these two media is, at least in part, due to a difference in amino acid concentrations. Thus the data indicate that in CHO cells cultured with abundant amino acids, GLUT4 accumulates in a peripheral compartment that is rapidly mobilized after insulin addition. Conversely, in low amino acid concentrations, GLUT4 may be targeted primarily to the endosomal system or the trans-Golgi reticulum. It is shown that rapamycin can inhibit the ability of amino acids to cause GLUT4 accumulation in a highly insulin-responsive compartment in CHO cells. Finally, it is demonstrated that amino acid concentrations also modulate GLUT4 trafficking in 3T3-L1 adipocytes, and that this effect is also rapamycin-sensitive.

An Assay for GLUT4 Trafficking at the Cell Surface

Several assays for GLUT4 trafficking at the cell surface have been described. The subcellular fractionation protocol pioneered by Cushman and colleagues provided the first evidence that glucose uptake is regulated by redistribution of glucose transporters to the plasma membrane (Cushman S. W., and L. J. Wardzala, 1980, J Biol Chem 255; 4758-62; Suzuki, K., and T. Kono, 1980, PNAS USA 77:2542-5). However, the use of subcellular fractionation to measure cell-surface GLUT4 is laborious, and accurate quantitation has been difficult because of cross-contamination of plasma membrane fractions (Holman, G. D., and S. W. Cushman, 1996, Semin Cell Dev Biol 7:259-268). The use of photoactivatable bis-mannose compounds to selectively tag cell surface glucose transporters allowed for improved quantitation, as well as measurement of steady-state internalization and externalization rate constants in the basal and insulin-stimulated states (Calderhead, D. M., et al., 1990, J Biol Chem 265:13801-8; Clark, A. E, et al., 1991, Biochem J 278:235-41; Holman, G. D., and S. W. Cushman, 1996, Holman, G. D., et al., 1990, J Biol Chem 265:18172-9, Satoh, S., H. et al., 1993, J Biol Chem 268:17820-9; Yang, J., and G. D. Holman, 1993, J Biol Chem 268:4600-3). Yet, this approach, too, is laborious and requires quantitative immunoprecipitation and analysis by SDS-PAGE. The preparation of plasma membrane sheets, followed by immunostaining for GLUT4 and fluorescence microscopy, is at best semi-quantitative (Katagiri, H., et al., 1996, J Biol Chem 271:16987-90; Maculay, S. L., et al., 1997 Biochem J 324:217-24; Marsh, B. J., et al. 1995 J Cell Biol 130:1081-91; Robinson, L. J., et al. 1992 J Cell Biol 117:1181-1196).

Expression of an exogenous, tagged GLUT4 reporter offers greater flexibility in detection and quantitation. Ebina and others have shown that an epitope tag in the first exofacial loop allows detection of a GLUT4 reporter on the surface of intact cells, and that changes in the amount present can be easily quantified (Kanai, F., et al., 1993, J Biol Chem 268:14523-6; Quon, M. J., et al. 1994 PNAS USA 91:5587-91;108). Other investigators fused GLUT4 to GFP, and observed insulin-regulated trafficking in individual cells by fluorescence microscopy (Dobson, S. P., et al. 1996 FEBS Lett 393:179-84; Oatey, P. B., et al. 1997 Biochem J 327:637-42; Thurmond, D.C., et al., 1998 J Biol Chem 273:33876-83). The use of these tags in combination has been described (J. S. Bogan and H. F. Lodish, Abstr, 38^{th} Annual Meeting of the American Society for Cell Biology, abstr. L65, 1998, and Lampson, M. A., et al., 2000 J Cell Sci 113:4065-4076). The level of expression of such reporter proteins is critical; significant overexpression of a GLUT4 reporter in primary rat adipose cells results in saturation of the trafficking mechanism, with decreased insulin responsiveness (Al-Hasani, H., et al. 1999 FEBS Lett 460:338-42). Indeed, significant overexpression in 3T3-L1 adipocytes might be one reason that insulin stimulated a relatively low increase in cell surface epitope-tagged GLUT4 in the original report (Kanai, F., et al., 1993, J Biol Chem 268:14523-6).

Presented herein is the first detailed characterization of a GLUT4 reporter with both an exofacial epitope tag and GFP fused to the cytosolic tail. The assay described represents a significant advance over previous metrics because it allows accurate quantification of changes in the proportion—rather than the amount—of GLUT4 that is present at the cell surface. These measurements are made on a cell-by-cell basis using flow cytometry, with the result that alterations in cell-surface GLUT4 targeting are determined with high specificity and precision. Importantly, it is shown that this reporter protein codistributes with native GLUT4, that native GLUT4 coimmunopurifies with vesicles containing the reporter, and that the reporter is reintemalized after insulin removal and recycles to the plasma membrane upon restimulation. The time course for GLUT4 reintemalization is slightly prolonged in 3T3-L1 adipocytes as compared to fibroblasts; this may be because adipocytes express a greater number of insulin receptors, which are endocytosed with bound insulin (Reed, B. C., et al. 1980 PNAS USA 77:285-9). Thus, insulin removal may not effectively stop insulin signaling in adipocytes. Careful isolation of a stable population of cells that express a moderate amount of the reporter protein is necessary, so as to avoid saturation of the trafficking mechanism. The use of flow cytometry, as well as the presence of several, tandem epitope tags, nonetheless enables the measurement of small amounts of cell-surface and total reporter. Finally, the method allows rapid analysis of multiple samples, making possible the detailed kinetic studies presented here.

Kinetics of GLUT4 Mobilization after Insulin Addition

Using this novel, FACS-based assay to measure the relative proportion of GLUT4 at the cell surface, detailed kinetic data is presented characterizing the insulin-responsiveness of various cell types. In 3T3-L1 preadipocytes and adipocytes, as well as in CHO cells, insulin causes a rapid ˜5-fold increase in plasma membrane GLUT4, peaking at about 5 minute. Cell surface GLUT4 then declines until a steady-state level is reached at about 15 minute This transient “overshoot” was also detected in cell-surface GLUT4 using uninfected 3T3-L1 adipocytes and subcellular fractionation to study the endogenous protein. In the fractionation data, the peak occurs slightly later (7 minutes), possibly because of the difficulty in synchronizing the responses of a large number of cells (several 10 cm dishes) required for biochemical analysis. By comparison, the FACS-based assay is done in a 6-well plate format. It seems more likely that all of the cells in a single well will behave as a synchronized cohort; if so, the FACS data may be the more accurate kinetic measurement.

A second possible explanation for the slightly faster kinetics observed using the FACS-based assay compared to subcellular fractionation could be transient trafficking of GLUT4 through caveolae. GLUT4 present in these triton-insoluble plasma membrane domains might not be detected using the described fractionation and immunoblotting protocol, yet an externalized myc epitope tag would presumably still be detected by cell-surface staining of intact cells. Whether or not GLUT4 traffics through caveolae remains uncertain, and data have been reported both in favor of and against this possibility (Gustavsson, J., et al. 1996 Mol Med 2:367-72, Kandror, K., V., et al. 1995 J Cell Biol 129:999-1006; Scherer, P. E., et al. 1994 J Cell Bio 127:1233-43). Recent work indicates that mobilization of GLUT4 requires signaling through a CAP-Cb1 complex that localizes transiently in triton-insoluble, caveolin-enriched plasma membrane subdomains after insulin stimulation (Baumann, C. A., et al. 2000 Nature 407:202-7). These data are broadly consistent with the notion that GLUT4 may also traffic transiently through such domains during insulin-stimulated exocytosis. Such a phenomenon might also help to explain observations that GLUT4 may under some circumstances be present in the plasma membrane without corresponding increases in glucose uptake (Hausdorf, S. F., et al., 1999, J Biol Chem 274:24677-84, Karnieli, E., et al. 1981 J Biol Chem 256:4772-7; Vannucci, S. J., et al. 1992 Biochem J 288:3525-30; Yang, J., et al. 1992 Biochem J 281:809-17).

The data could be consistent with a biphasic effect on either GLUT4 exocytosis or endocytosis. The data favor the former interpretation because of biochemical and immunoelectron microscopy data indicating that in adipocytes, GLUT4 is sequestered from endosomes into a highly insulin-responsive, TfnR-negative and CD-MPR-negative pool (Aledo, J. C. et al., 1997, Biochem J. 325:727-32; Hashiramoto. M. and James. D. E., 2000, Mol Cell Biol 20:416-27; Kandror, K. V. and Pilch. P. F., 1998, Biochem J 331:829-35; Lee, W. et al., 1999, J Biol Chem 274:37755-62; Martin, S. J. et al., 1997, J Cell Sci 110:2281-2291; Ramm, G. et al., 2000, Mol Biol Cell 11:4079-91). Insulin acts primarily to mobilize this sequestered pool of GLUT4 to the cell surface. Thus in the absence of insulin, the exocytosis rate is limited by sequestration and accumulation of GLUT4 in the insulin responsive compartment. After insulin addition, the GLUT4 that has accumulated in this compartment is mobilized, and the compartment itself is depleted of GLUT4. Importantly, the rate of GLUT4 exocytosis in the steady-state presence of insulin is now limited at some other step in the recycling pathway; this could be trafficking from endosomes to the insulin responsive compartment or directly to the cell surface (Holman, G. D., et al., 1994, J Biol Chem 269:17516-24; Yeh, J. I., et al., 1995, Biochem 34:15523-31). Recent analyses also suggest the presence of a unique GLUT4 storage compartment, and suggest that traffic through this compartment to the highly insulin-responsive exocytic compartment may be rate limiting for exocytosis in the steady-state presence of insulin (Lee, W., et al., 1999, J Biol Chem 274:37755-62; Lee, W., et al., 2000, Biochem 39:9358-66). This notion may fit with other recent work demonstrating that GFP-tagged GLUT4 proteins move to the cell surface directly from the perinuclear region, which may be a storage compartment, in 3T3-L1 adipocytes (Patki, V. V., et al., 2001, Mol Biol Cell 12:129-141). In either case, the relative amount of GLUT4 present in the highly insulin-responsive compartment of unstimulated cells can be assessed indirectly, as the amount of GLUT4 translocated immediately (in the first 5 minutes) after insulin addition (i.e. before steady-state is reached).

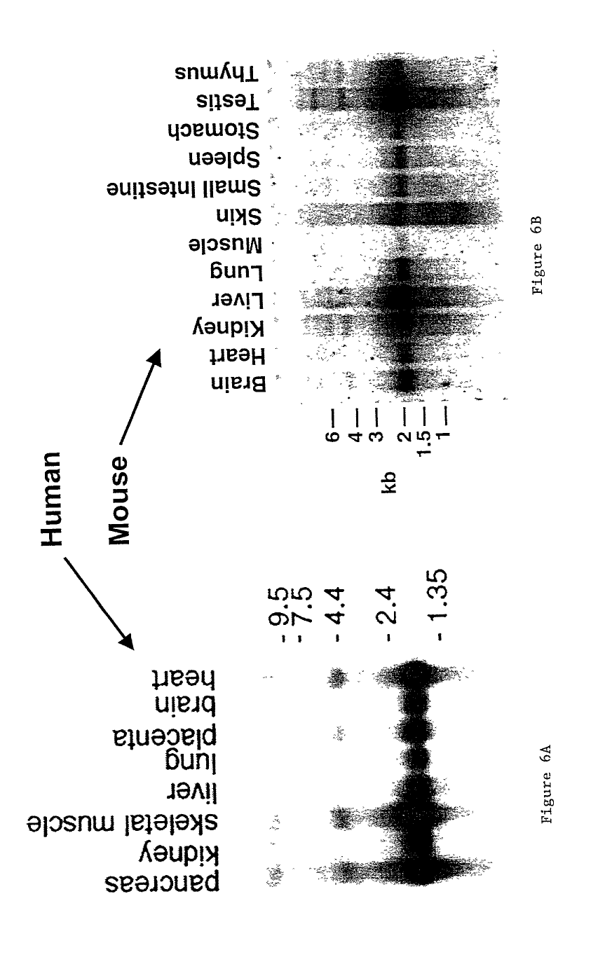

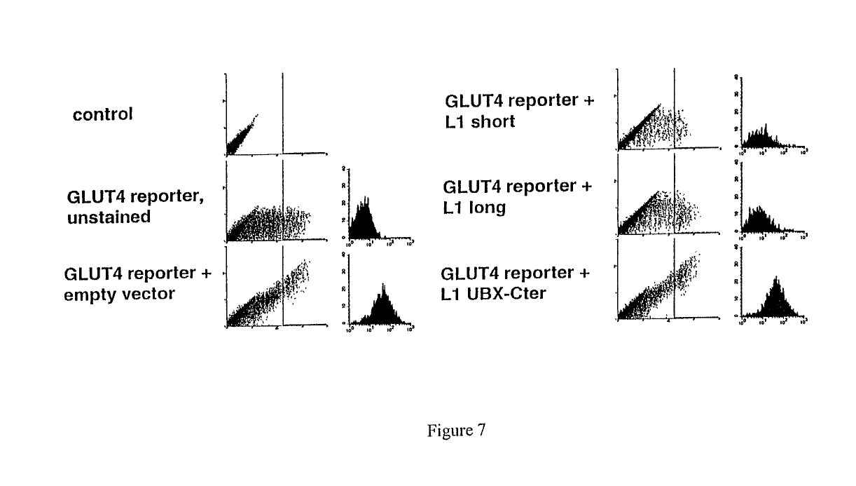

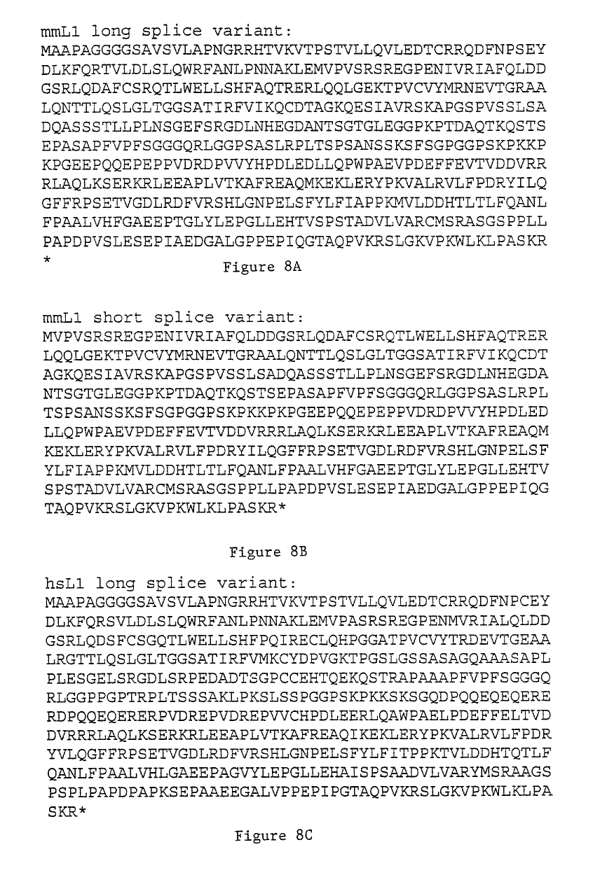

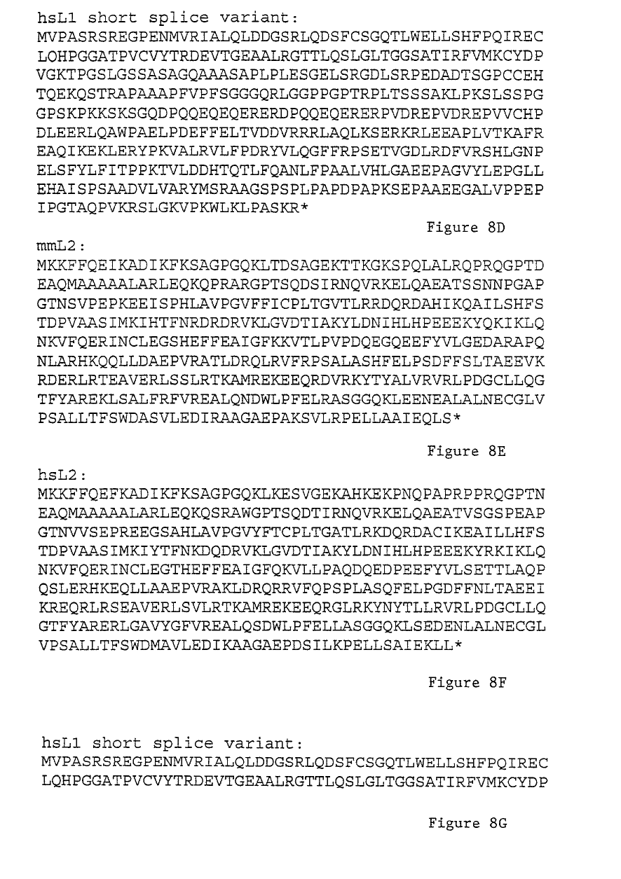

The above reasoning forms the rationale for our focus on the biphasic kinetics of GLUT4 translocation in insulin-responsive cells. The first phase of the response (the “overshoot” before the steady-state) represents mobilization of GLUT4 that has accumulated in the insulin responsive pool. The second phase (the steady-state in the presence of insulin, after 15-20 min.) is determined by trafficking rates that do not inform us as to the initial size of the insulin responsive pool. The initial overshoot of the steady-state GLUT4 distribution after insulin stimulation was predicted by mathematical analysis, but measurement of GLUT4 or IRAP in plasma membranes by subcellular fractionation, or photolabeling did not convincingly demonstrate its occurrence (Clark, A. E, et al., 1991, Biochem J 278:235-41; Piper, R. C. et al., 1991, Am J Physio 260:C570-80; Holman, G. D. et al., 1994, J Biol Chem 269:17516-24; Ross, S. A., et al., 1998, Biochem J 330:1003-8; Satoh, S., H. et al., 1993, J Biol Chem 268:17820-9). Likewise, older studies of glucose uptake and cytochalasin B binding failed to detect a biphasic response (Karnieli, E., et al. 1981 J Biol Chem 256:4772-7; Kohanski,R. A., et al. 1986 J Biol Chem 261:12272-81, Kuroda, M., et al. 1987 J Biol Chem 262:245-53). Our observation of this phenomenon may reflect an improved sensitivity of our FACS assay. However, this initial “overshoot” was also detected in cell surface GLUT4 using uninfected 3T3-L1 adipocytes and subcellular fractionation. It is unknown why this response has not been detected previously, though clearly the details of insulin stimulation are important. In experiments examining native GLUT4, high concentrations of insulin added from a prewarmed 3×stock were utilized to simultaneously and maximally stimulate all of the cells in the population. Cells would probably not have been stimulated in this manner were it not for the FACS results, which were being confirmed. Another consideration is that the divergent results may be due to differences between 3T3-L1 cells and primary rat adipocyte, which were used for many previous kinetic studies. The measurement of the initial rate of GLUT4 externalization is more rapid than reported by Clark et al. and Patki et al., but is quite similar to data of Satoh et al. (Clark, A. E, et al., 1991, Biochem J 278:235-41, Patki, V. V., et al., 2001, Mol Cell Biol 12:129-141; Satoh, S., H. et al., 1993, J Biol Chem 268:17820-9). Indeed, careful examination of the latter group's data suggests a slight overshoot of the insulin-stimulated steady-state response in primary rat adipocytes, through at the time this appears to have been attributed to uncertainly in the measurement (see FIG. 6A of Satoh, S., H. et al., 1993, J Biol Chem 268:17820-9).

Cell-type Specificity of Glut4 Trafficking

Using the assay described here, highly insulin-responsive GLUT4 trafficking in differentiated and undifferentiated 3T3-L1 cells and in CHO cells were initially observed, leading to the suggestion that machinery required for insulin-responsive GLUT4 trafficking might not be exclusive to adipose and muscle (J. S. Bogan and H. F. Lodish, Abstr. 38th Annual Meeting of the American Society for Cell Biology, abstr. L65, 1998). Very recently, similar results led others to conclude that undifferentiated fibroblasts possess the requisite mechanism (Lampson, M. A., et al. 2000 J Cell Sci 40113:4065-4076). The data demonstrate that this is not the case. It was found that a highly insulin-responsive pool containing GLUT4 was present in fully differentiated 3T3-L1 adipocytes, but is absent in NIH 3T3 cells. This conclusion is based as much on the presence of the overshoot as it is on the magnitude of the increase in cell surface GLUT4 after insulin addition to 3T3-L1 adipocytes. After insulin treatment of NIH 3T3 cells, neither the overshoot nor a large increase in cell-surface GLUT4 was observed, consistent with the generally held notion that a highly insulin-responsive mechanism is not present ubiquitously (Czech, M. P., et al., 1993, J Cell Biol 123:127-35; Czech, M. P., et al., 1993, J Cell Biol 123:127-35; Haney, P. M., et al., 1991, J Cell Biol 689-99; Herman, G. A., et al., 1994, PNAS USA 91:12750-4; Hudson, A. W, et al., 1993, J Cell Biol 122:579-88; Hudson, A. W, et al., 1992, J Cell Biol 116:785-97; Ross, S. A., et al., 1996, J Biol Chem 271:3328-32; Schurmann, A., et al., 1992, Biochim Biopys Acta 1131:245-52; Verhey, L. J., et al., 1993, J Cell Biol 123:137-47).

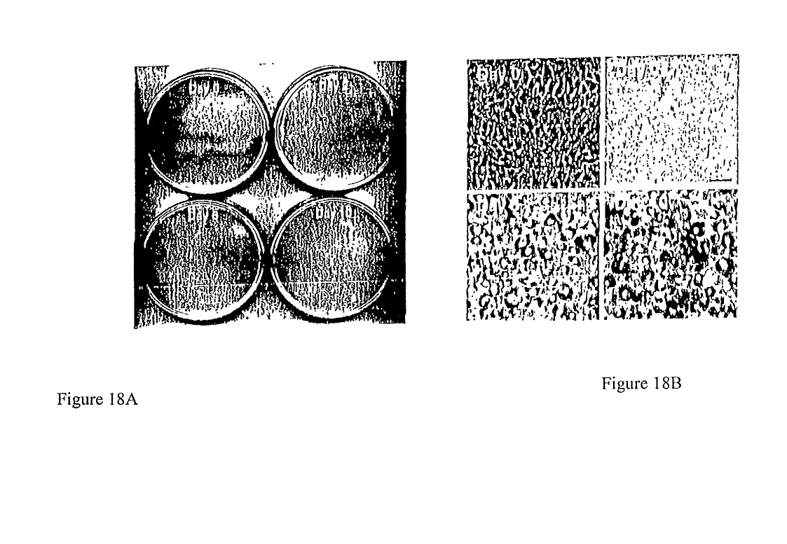

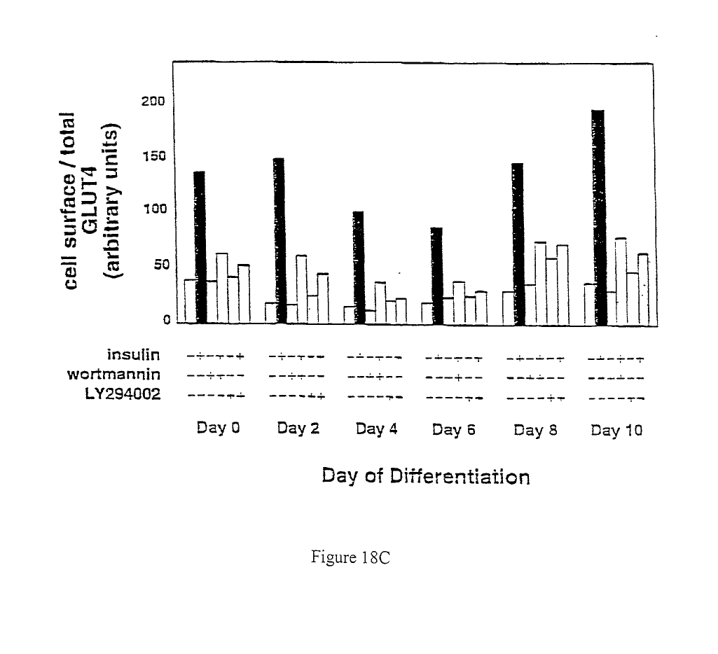

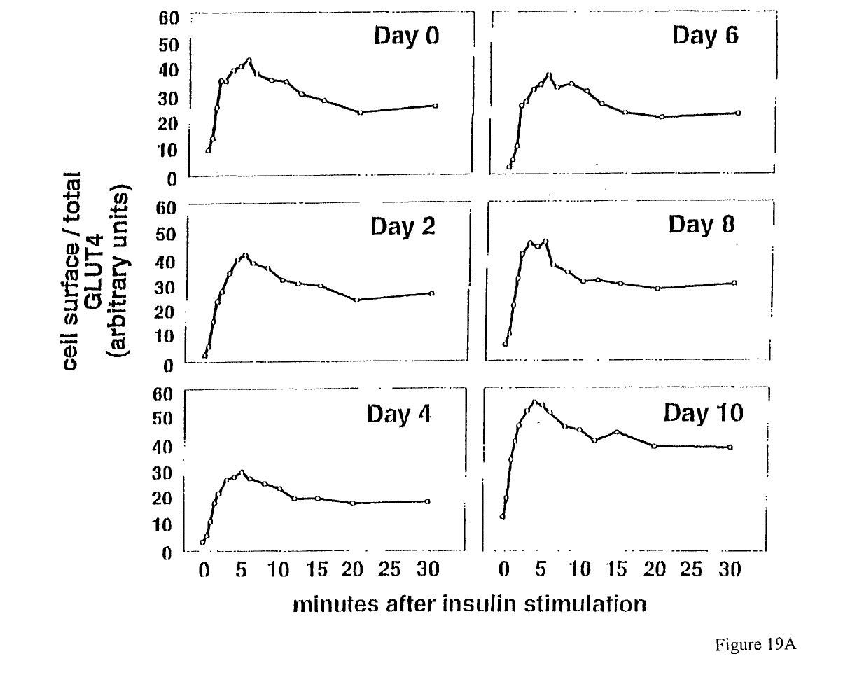

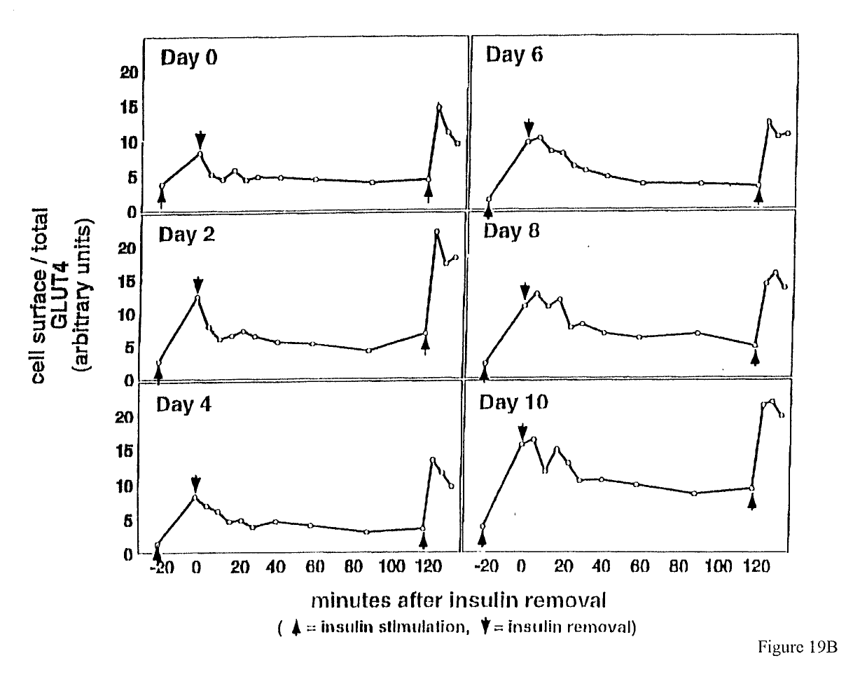

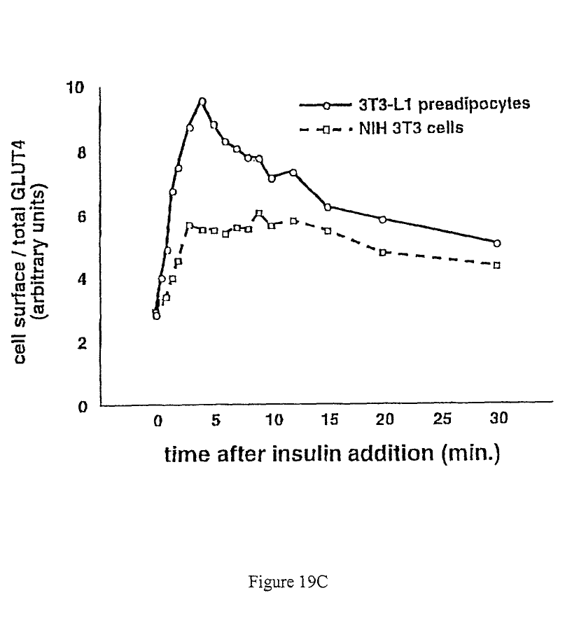

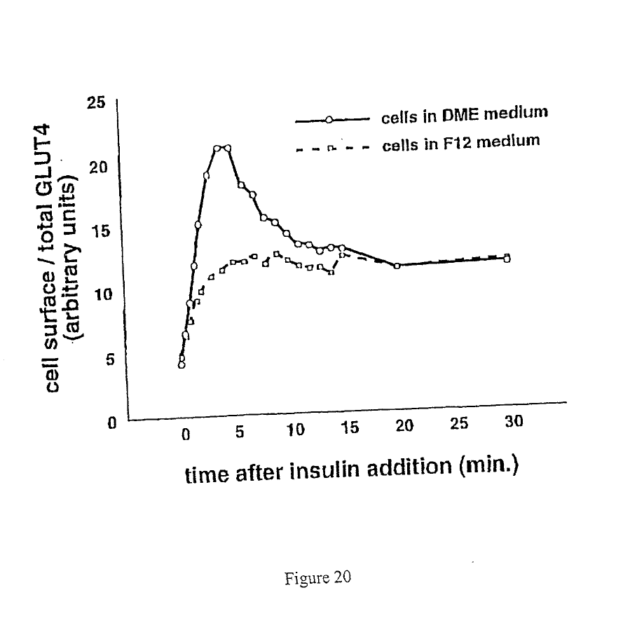

The situation in 3T3-L1 preadipocytes and in CHO cells is more complicated. Highly insulin-responsive GLUT4 trafficking was observed, at least to some degree, in “undifferentiated” 3T3-L1 preadipocytes. The overall “fold increase” of GLUT4 at the cell surface is less in these cells than in Day 2 3T3-L1 cells or in fully differentiated 3T3-L1 adipocytes, yet consistent observation of an overshoot of the final, steady-state response in the presence of insulin (FIGS. 18B, 19A and 20). This observation does not result from culture of the cells in fetal bovine serum rather than calf serum (FIG. 20). Greater sequestration of the GLUT4 reporter on Day 2 of differentiation (and subsequently) than in the “undifferentiated” cells is consistently observed (FIGS. 18B and 19A). It is possible that two mechanisms are operative: one for basal sequestration (that is active in 3T3-L1 adipocytes, but not active in 3T3-L1 preadipocytes) and another that is responsible for the overshoot (that is active in both 3T3-L1 adipocytes and preadipocytes, but not in NIH 3T3 cells). However, a simpler explanation is that GLUT4 undergoes partial targeting to a highly insulin-responsive compartment in the 3T3-L1 preadipocytes, sufficient to cause the overshoot but not sufficient to cause significant basal sequestration (i.e., by drawing enough GLUT4 out of the endosomal system). It can be hypothesized that such a mechanism becomes more active at Day 2 of differentiation, and is then sufficient to deplete GLUT4 from endosomes. This would result in greater “fold translocation” of GLUT4 to the cell surface on Day 2 because of increased sequestration in the basal state, consistent with the data. Such an interpretation might also be compatible with findings that a biochemically detectable population of highly insulin-responsive vesicles first develops in 3T3-L1 cells at 2 to 3 days after induction of differentiation (Czech, M. P., et al., 1993, J Cell Biol 123:127-35). Of course, there is also likely to be significant variation among 3T3-L1 cell lines used in different laboratories.

The idea of partial sorting may also apply to CHO cells, which appear to possess a highly-insulin sensitive trafficking mechanism, but which do not generally translocate GLUT4 by the same “fold-increase” seen in fully differentiated 3T3-L1 adipocytes (Asano, T., et al., 1992, J Biol Chem 267:19636-41; Czech, M. P., and S. Corvera, 1999, J Cell Biol 123:127-35; Johnson, A. O. et al., 1998, J Cell Biol 273:17968-77; Kanai, F., et al., 1993, J Biol Chem 268:14523-6; Shibaski, Y. T., et al., 1992, Biochem J 281:829-34). Importantly, CHO cells have several adipocyte-like features, and the observation of a highly insulin-responsive GLUT4 trafficking mechanism in these cells does not imply that such a mechanism is present in all cell types. CHO-K1 cells transfected with the β3-adrenergic receptor accumulate triglyceride droplets when cultured in differentiation media similar to that used for 3T3-L1 cells (Gros, J., et al., 1999, J Cell Sci112:3791-7). The untransfected CHO cells constitutively express hormone sensitive lipase and PPARγ, a major regulator of adipose differentiation; PPARγ expression is upregulated in the presence of the β3-adrenergic receptor and differentiation medium. Thus, CHO cells have several adipocyte-like characteristics, and the notion the CHO cells can mobilize GLUT4 from an adipocyte-like, highly insulin-responsive compartment is not inconsistent with reports finding that heterogeneous expression of GLUT4 usually results in intracellular sequestration without insulin-responsiveness (Asano, T., et al., 1992 J Biol Chem 267:19636-41; Czech, M. P., et al., 1993, J. Cell Bio 123:127-35; Haney, P. M., et al., 1991, J. Cell Bio 114:689-99, Herman, G. A, et al., 1994, PNAS USA 91:12750-4; Schurmann, A. et al., 1992 Biochem Biophys Acta 1131:245-52; Verhey, K. J. et al., 1993, J Cell Biol 123:137-47).

Amino Acid Concentrations Regulate GLUT4 Trafficking

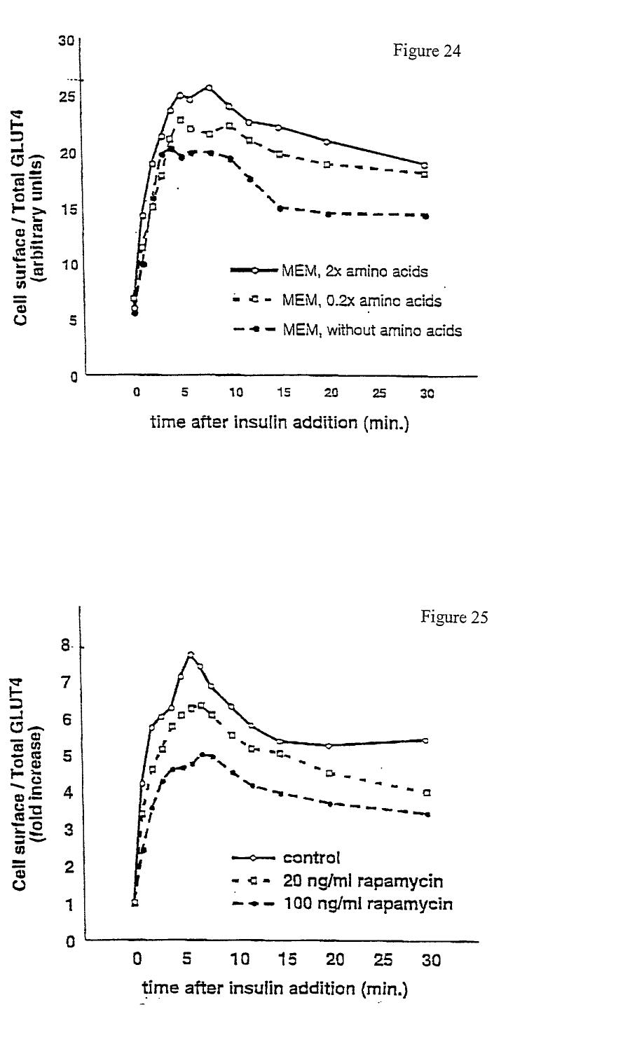

It can be hypothesized that the mechanism for sorting and retaining GLUT4 in a highly insulin-responsive compartment is somewhat less efficient in CHO cells than in 3T3-L1 adipocytes, but that it is otherwise essentially the same and that both cell types can be considered as models for primary adipocytes. 3T3-L1 adipocytes are the better model, based on expression of known adipocyte marker proteins. Despite this distinction, amino acid concentrations (and rapamycin treatment) likely alter the same step(s) in the GLUT4 recycling pathway in both cell types. If so, then the simplest explanation to encompass the data presented is that amino acid concentrations (and rapamycin) alter the rate of GLUT4 movement from the highly insulin-responsive compartment to the cell surface, both in the absence and presence of insulin. In CHO cells, most GLUT4 recycles via the endosomal system. Yet in sufficient amino acids, traffic through the highly insulin-responsive compartment becomes significant and is detectable (FIG. 22). In contrast, in 3T3-L1 adipocytes the sorting/retention machinery is efficient enough to cause some GLUT4 accumulation in—and trafficking through—a highly insulin-responsive compartment even in low concentrations of amino acids. Thus, in the presence of low amino acid concentrations or rapamycin, the overshoot remains, though the overall “fold increase” in cell surface GLUT4 is decreased (FIGS. 24 and 25).

The kinetic and microscopy data suggest that amino acid concentrations regulate the accumulation of GLUT4 in a peripheral, highly insulin-responsive compartment in CHO cells. This in the presence of high amino acid concentrations, GLUT4 is both concentrated in the periphery and insulin stimulation results in an initial, marked overshoot of the steady-state proportion of GLUT4 at the cell surface. In low amino acid concentrations much GLUT4 remains in a perinuclear or trans-Golgi location, and the kinetics of externalization are more modest and exhibit no overshoot of the final, steady-state response. It is not known if this peripheral, insulin-mobilizable compartment in CHO cells is the same as the highly insulin-responsive compartment in 3T3-L1 cells. Indeed, immunofluorescence microscopy in primary and cultured adipocytes suggests that TfnR-negative compartments containing GLUT4 are present both in the periphery and in a perinuclear location (Bogan, J. S., and H. F. Lodish, 1999, J Cell Biol 146:609-20; Malide, D., and S. W. Cushman, 1997, J Cell Sci 110:2795-806; Malide, D., et al., 1997, J Histochem Cytochem 45:1083-96). Perinuclear GLUT4 may be in a storage compartment, and appears to require intact actin and microtubule networks for mobilization (Lee, W., et al., 1999, J Biol Chem 274:37755-62; Lee, W., et al., 2000, Biochem 39:9358-66; Patki, V. V., et al., 2001, Mol Cell Biol 12:129-141; Lee, W., et al., 2000, Biochem 39:9358-66). It may be that insulin stimulates both release of a GLUT4 tethering mechanism, as well as fusion of vesicles containing GLUT4 with the plasma membrane, and that low amino acid concentrations (or rapamycin) inhibit the latter, but not the former. If such an explanation is correct, it would suggest that the peripheral vesicles we observe in CHO cells cultured in DMEM may be docked, but not fused, at the plasma membrane.

That amino acid abundance (or rapamycin treatment) would secondarily regulate a step in the GLUT4 recycling pathway that is also controlled by insulin fits well with other data. Rapamycin appears to inhibit mTOR kinase activity quite specifically, and mimics amino acid starvation in both yeast and mammalian cells; in yeast Tor protein activates metabolic pathways for glucose utilization (Burnett, p., E. et al., 1998, PNAS USA 95:1432-7, 29, reviewed in Schmelzle, T. and M. N. Hall, 2000, Cell 103:253-62). In mammalian cells, insulin is well known to signal through phosphatidylinositol-3-kinase and PKB/Akt to phosphorylate p70 S6 kinase and eIF-4E BP1 and this effect is sensitive to both amino acid sufficiency and rapamycin (Hara, K., et al., 1998, J Biol Chem, 273:(34):2216058, Scott, P. H., et al., 1998, Biochim Biophys Acta 1121:245-52; Patti, M. E., et al., 1998, J Clin Invest 101:1519-29). Paradoxically, amino acids appear to inhibit insulin-stimulated phosphorylation of IRS-1 and IRS-2 and inhibit phosphatidylinositol 3-Kinase activity (Shigemitsu, K., et al., 1999, J Biol Chem 274:1058-65). This latter effect may result from mTOR-mediated serine phosphorylation and subsequent proteasomal degradation of IRS proteins (Hartman, M. E., et al, 2001, Biochem Biophys Res Commun 280:776-781; Haruta, T., et al., 2000, Mol Endocrinol 14:783-94; Pederson, T. M., et al., 2001, Diabetes 50:24-31). A rapamycin-sensitive pathway is also important in controlling expression of the p85α regulatory subunit of phosphatidylinositol 3-kinase in muscle (Roques, M. and H. Vidal, 1999, J Biol Chem 274:34005-10). Other data indicate that rapamycin and nutrient insufficiency decrease signaling through atypical protein kinase C (Parekh, D., et al., 1999, J Biol Chem 274:34758-64; Ziegler, W., H., et al., 1999, Curr Biol 9:5222-9). Since both PKB/Akt and atypical protein kinase C isoforms have been implicated in regulation of GLUT4 trafficking, either or both of these pathways could be important for the response of GLUT4 to amino acid concentrations described (Czech, M. P., and S. Corvera, 1999, J Biol Chem 274:1865-8). The data do not contradict previous work showing that short-term rapamycin treatment has no effect on insulin-stimulated glucose uptake (Fingar, D. C., et al., 1993, J Biol Chem 268:3005-8). Rather, an effect of longer-term amino acid starvation or rapamycin treatment on GLUT4 distribution in unstimulated cells was observed. Thus, it seems more likely that amino acids and rapamycin are regulating some transcriptional or translational output, which in turn alters the distribution of GLUT4 in the basal state.

### EXEMPLIFICATION

The Materials and Methods Described were Utilized in the Examples which Follow.

Antibodies and Reagents

Cell culture media and supplements were purchased from Life Technologies (Grand Island, N.Y.) and JRH Biosciences (Lenexa, Kans.). Anti-c-myc monoclonal antibody (clone 9E10) was from Babco/Covance (Richmond, Calif.) and from Roche. An anti-insulin receptor β-chain antibody was purchased from BD Transduction Laboratories. Normal Donkey serum and R-phycoerythrin conjugated donkey F(ab′)₂ anti-mouse IgG secondary antibody were purchased from Jackson Immunoresearch (West Grove, Pa.). Restriction enzymes were from New England Biolabs (Beverly, Mass.) and Pfu and Taq DNA polymerases were from Stratagene (La Jolla, Calif.). Wortmannin, LY294002, and rapamycin were from Calbiochem (La Jolla, Calif.). Oil Red O and other chemicals were from Sigma (St. Louis, Mo.).

Cell Culture

Murine 3T3-L1 fibroblasts were cultured in DMEM containing 10% fetal bovine serum (or 10% calf serum, where noted) and differentiation was induced according to established protocol (Bogan, J. S., and H. F. Lodish, 1999, J Cell Biol 146:609-20; Frost, S. C., and M. D. Lane, 1985, J Biol Chem 260:2646-52). Briefly, cells were allowed to reach confluence at least two days prior to the induction of differentiation. Differentiation was induced (on “Day 0”) with medium containing 0.25 μM dexamethasone, 160 nM insulin, and 500 μM methylisobutylxanthine. After 48 hours (“Day 2”), the cells were fed with medium containing 160 nM insulin. After an additional 48 hours (“Day 4”), the cells were fed every two days with DMEM/10% fetal bovine serum. All media were supplemented with 2 mM glutamine, 100 U/ml penicillin, and 0.1 mg/ml streptomycin. Differentiation was monitored by noting the accumulation of lipid droplets, which typically began by Day 4 of differentiation. Cells were considered fully differentiated between Days 8 and 12. The terms “Day 0 3T3-L1 cells” and “confluent 3T3-L1 preadipocytes” as used herein are interchangeable.

CHO-K1 cells stably expressing the murine ecotropic retroviral receptor were kindly provided by Drs. David Hirsch, Roger Lawrence, and Monty Kreiger (Massachusetts Institute of Technology, Cambridge, Mass.), and were maintained in Ham's/F12 medium (“F12”) with 10% fetal bovine serum, 2 mM glutamine, 100 U/ml penicillin, and 0.1 mg/ml streptomycin (Baker, B. W., et al., 1992, Nuc Acids Res 20:5234). NIH 3T3 cells were cultured in DMEM containing 10% calf serum, glutamine, penicillin and streptomycin as above. Phoenix ecotropic retroviral packaging cells were a gift from Dr. Garry Nolan (Stanford University Medical Center) and VE23 ecotropic retroviral packaging cells were a gift from Dr. Merav Socolovsky (Whitehead Institute, Cambridge Mass.) (Kinoshita, S., et al., 1998, Cell 95:595-604; Socolovsky, M., et al., 1997, J Bio Chem 272:14009-12; Swift, S. E., et al, (ed) 1998, Current Protocols in Immunology, vol. 2, John Wiley and Sons, Inc., New York). Both retroviral packaging cell lines were cultured in DMEM, 10% fetal bovine serum, glutamine, penicillin and streptomycin as above.

For experiments involving culture in MEM with various amino acid concentrations, MEM Select-Amine kit (Life Technologies) was used. The concentration of each amino acid designated as “1×” is as follows (free base, in mg/L): L-Arg, 102; L-Cys, 36; L-His, 30; L-Ile, 52; L-Leu, 52; L-Lys, 57; L-Met, 15; L-Phe, 32; L-Thr, 48; L-Trp, 10; L-Tyr, 35; L-Val, 46. Relative to these concentrations, DMEM contains 2×Cys, Ile, Leu, Lys, Met, Phe, Thr, Tyr, and Val, 1.6×Trp, 1×His, and 0.67×Arg. F12 contains 1.67×Arg, 1.25×Cys, 0.5×His, Lys, 0.3×Met, 0.25×Leu, Thr, Val, 0.2×Trp, 0.15×Phe, Tyr, and 0.08×Ile. All media contained 2 mM glutamine, as well as penicillin and streptomycin as above.

Construction of a GLUT4 Reporter

A human GLUT4 cDNA containing a c-myc epitope tag in the first exofacial loop was kindly provided by Drs. Zhijun Luo and Joseph Avruch (Massachusetts General Hospital, Boston, Mass.). This clone had been constructed as described by Kanai et al. (1993). The green fluorescent protein (GFP) coding sequence in frame was fused to the carboxy terminus of this GLUT4 clone based on the result of Dobson et al.(1996), that GLUT4-GFP appears to localize the traffic similarly to wildtype GLUT4. The GFP coding sequence from pEGFP-N1 (Stratagene) was first cloned into the pMX retroviral vector using EcoRI and NotI, to generate the plasmid pMX-GFP (Onishi, M., et al 1996 Exp Hematol 24:324-9). PCR was done using the primers 5′-GACATTTGACCAGATCTCGG-3′-(SEQ ID NO: 11) and 5′-GGCCCGCGGGTCATTCTCATCTGGCCC-3′ (SEQ ID NO: 12) to generate a ˜110 bp BgIII/SacII fragment from the 3′ end of the rat GLUT4 cDNA (Charron, M. J., et al., 1989 PNAS USA 274:3253-6). This PCR product and an EcoRI/BgIII fragment containing most of the GLUT4myc cDNA were used in a three-way ligation with EcoRI/SacII-digested pMX-GFP, to generate pMX-GLUT4 myc-GFP. Next, six additional myc epitope tags were added in tandem with the existing myc epitope tag, for a total of seven myc epitope tags. PCR was first utilized to amplify a ˜240 bp EcoRI/HindIII fragment including the 5′ end of the rat GLUT4 cDNA and part of a myc tag, using the primers 5′-CCGGCCGAATTCATGCCGTCGGGTTTCCAGCAGATC-3′ (SEQ ID NO: 13) and 5′-CTTCAGAAATAAGCTTTTGCTCCTCTGCAGGAC CCTGCCTACCCAGCCAAGTTGC-3′(SEQ ID NO: 14). This fragment was used to replace a corresponding fragment in pMX-GLUT4myc-GFP, creating a unique HindIII site within the myc epitope tag. A HindIII fragment containing six tandem myc epitope tags was amplified from the plasmid pCS2+MT, a gift of Bill Schiemann (Whitehead Institute, Cambridge Mass.), using the primers 5′-CCATCGATTTAAAGCTATGGAG CAAAAGCTTATTTCTGAAGAGG-3′ (SEQ ID NO: 15) and 5′-CAGAAATAAGCT TTTGCTCCTCTGCAGGCTCAAGAGGTCTTGAGTTCAAGTCCTCTTC-3′ (SEQ ID NO: 16). This fragment was inserted into the HindIII site of pMX-GLUT4myc-GFP, creating pMX-GLUT4myc7-GFP. The entire coding regions of the pMX-GLUT4myc-GFP and pMX-GLUT4myc7-GFP plasmids were verified by sequencing. The GLUT4myc7-GFP coding sequence was also placed in the pB retroviral vector in order to optimize the potential translation efficiency; pB is identical to pMX except that two point mutations were introduced to eliminate potential start codons 5′ of the cloning site (J. S. Bogan, X, Liu, A. E. McKee and H. F. Lodish, unpublished). The “GLUT4 reporter” as used herein refers to that encoded by the GLUT4myc7-GFP sequence.

Production of Retroviral Supernatant and Isolation of Infected Cell Populations

Phoenix or VE23 ecotropic packaging cells were transferred with pMX-GLUT4myc-GFP, pMX-GLUT4myc7-GFP, or pB-GLUT4myc7-GFP plasmids using calcium phosphate as described (Kinoshita, S., et al., 1998, Cell 95:595-604; Socolovsky, M., et al., 1997, J Bio Chem 272:14009-12; Swift, S. E., et al., (ed) 1998, Current Protocols in Immunology, vol. 2, John Wiley and Sons, Inc., New York). In some instances, Phoenix cells were transfected using Fugene 6 (Roche) as per the manufacturer's protocol. Media containing recombinant retroviruses were harvested 48 or 72 hours after transfection, and were used to infect dividing 3T3-L1 preadipocytes, NIH 3T3 cells, or CHO cells expressing the murine ecotropic receptor. For 3T3-L1 preadipocytes infected with pMX-GLUT4myc7-GFP, flow cytometry demonstrated the presence of GFP in >90% of cells after infection. Stable populations of infected cells expressing ‘high’, ‘medium’, or ‘low’ amounts of the reporter were isolated by flow sorting cells falling within narrow ranges of GFP fluorescence. The sorted cells were expanded, differentiated into adipocytes, and insulin-stimulated GLUT4 trafficking (stimulated/basal) was measured by flow cytometry in all cases. It was thought that there might be a trade-off between signal/noise (with low amounts of the reporter) and potential saturation of a trafficking mechanism (at high amounts of the reporter). In general, ‘medium’ or ‘high’ expressing cells were selected for use in subsequent experiments, since these had the greatest fold-increase in cell surface GLUT4 after insulin treatment. These cells generally contained 5 to 10 times as much reporter protein as native GLUT4 in the mature 3T3-L1 adipocytes, as judged by immunoblotting with antibodies directed against the N-and C-termini of GLUT4 (kindly provided by Dr. Maureen Charron, Albert Einstein College of Medicine, Bronx, N.Y.). Similar optimization of reporter protein expression levels was carried out in NIH 3T3 and CHO cells.

Measurement of Plasma Membrane GLUT4 Trafficking by Flow Cytometry

Confluent cells were reseeded on the indicated day of differentiation to six well plates (Coming, Costar #3506) one day before use in experiments, and were starved in DMEM without fetal bovine serum for at least 3 hours before insulin stimulation. Insulin was generally used at 160 nM; occasionally it was used at 80 nM or 200 nM and no difference was noted between these concentrations in either 3T3-L1 or CHO cells. Insulin was added directly to the wells from a 100×stock. After treatment in the presence or absence of insulin for the times indicated in each figure, cells were quickly transferred to 40° C. and washed with cold phosphate buffered saline (PBS) containing 0.9 mM Ca⁺⁺ and 0.5 mM Mg⁺⁺ (PBS++). All subsequent steps were carried out at 4° C., and staining of externalized myc epitope was done on adherent cells. Cells were incubated with a 1:200 dilution of anti-myc (9E10) ascites or using 25 μg/ml purified 9E10 in PBS++, 2% bovine serum albumin (BSA), 4% donkey serum for 1.5 hours. Cells were then washed twice in PBS++, for 5 minutes each time. They were then incubated 45 minutes in 12.5 to 25 μg/ml R-phycoerythrin conjugated donkey F(ab′)₂ anti-mouse IgG secondary antibody, diluted in PBS++, 2% BSA, 4% donkey serum. Cells were rinsed twice in PBS++, then washed three times in PBS++ for 10 minutes each time, and were resuspended by gentle scraping in PBS++, 2% BSA or PBS++, 5% fetal bovine serum for flow cytometry. For experiments involving insulin removal, cells were chilled as above after insulin stimulation, then washed twice with 5 mM sodium acetate, 150 mM NaCl, pH 4.0 (Kao, A. W., et al., 1998, J Biol Chem 273:25450-7; Yang., J., et al., 1992, J Biol Chem 267:10393-9). Cells were rewarmed in 37° C. DMEM for the times indicated, restimulated or not with insulin, and then returned to 4° C. and chilled with cold PBS++ before staining as above.

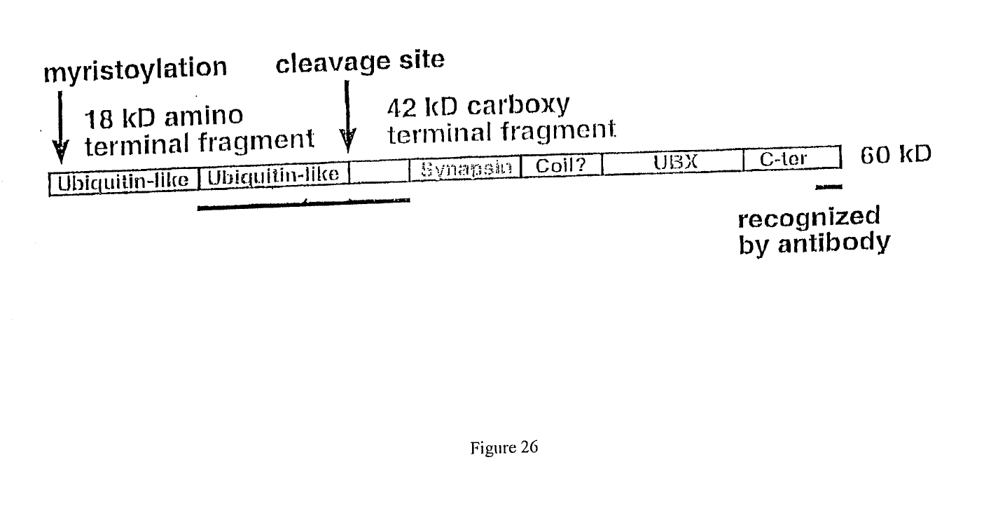

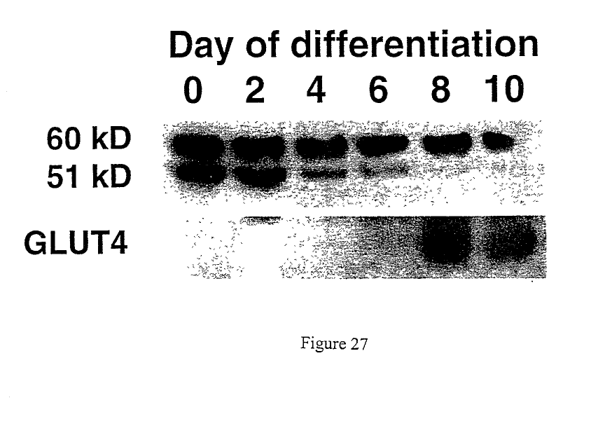

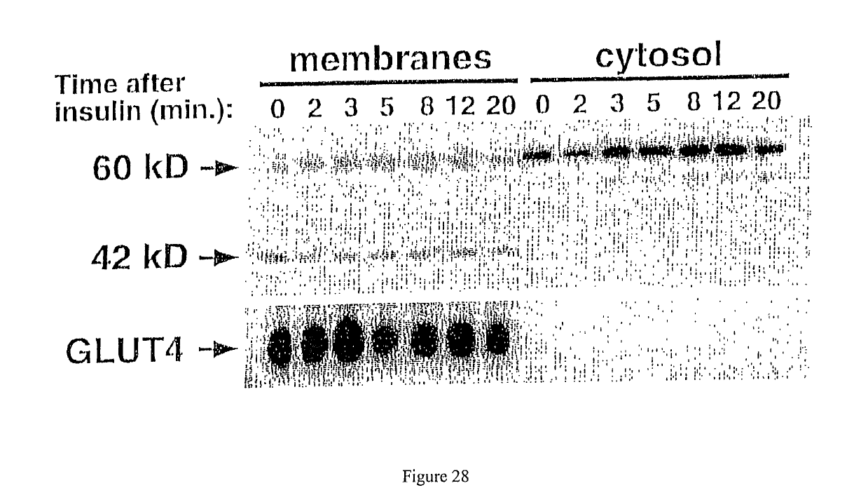

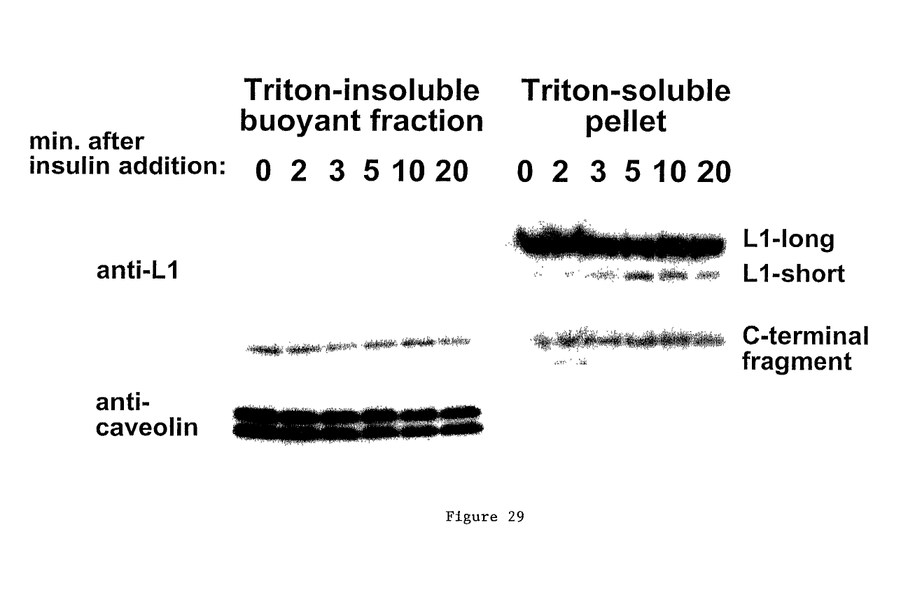

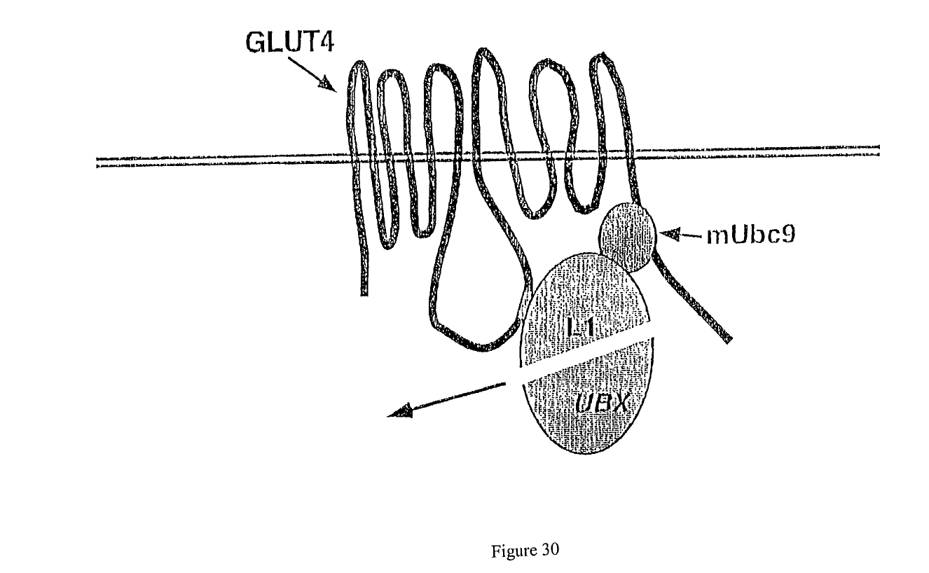

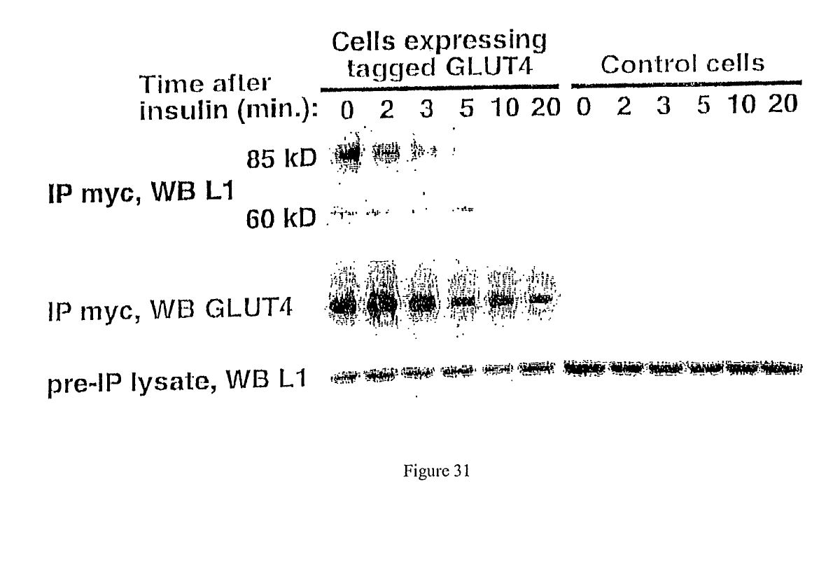

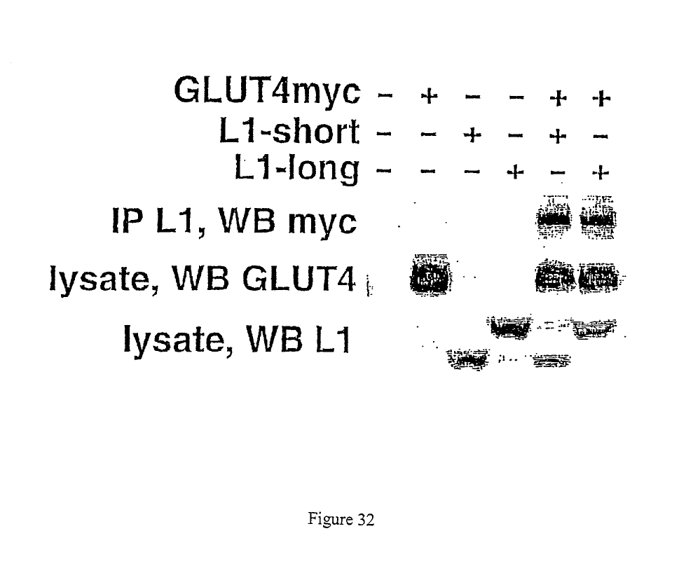

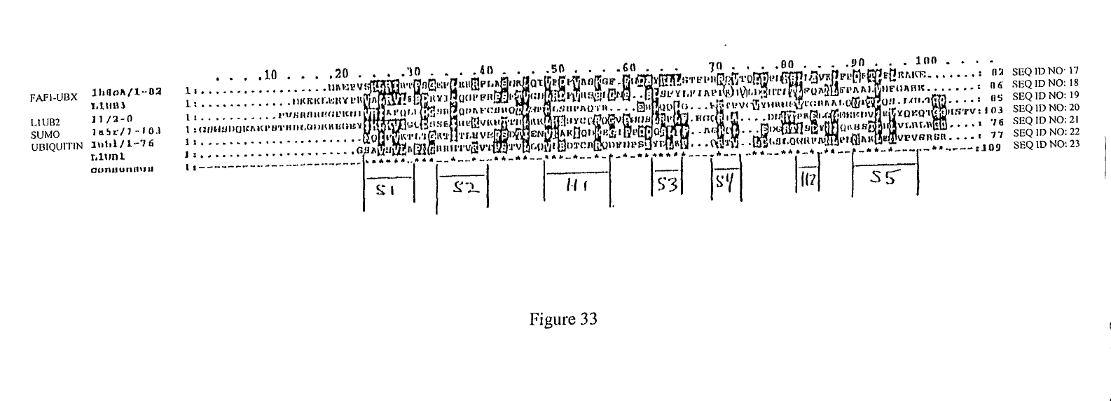

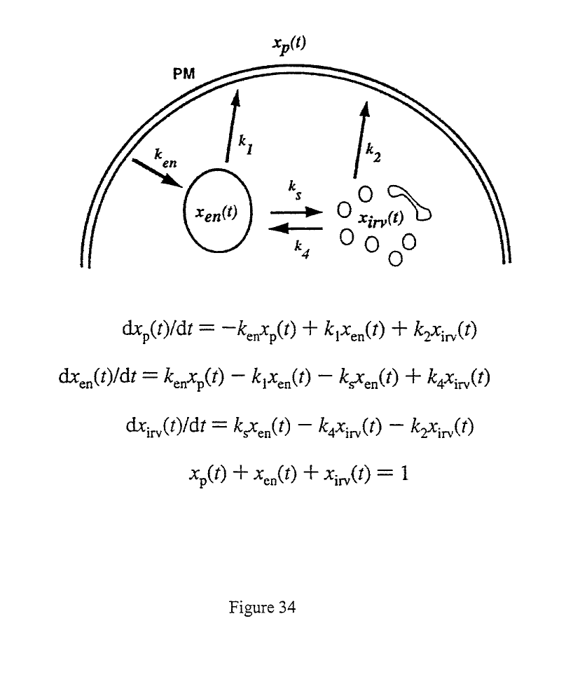

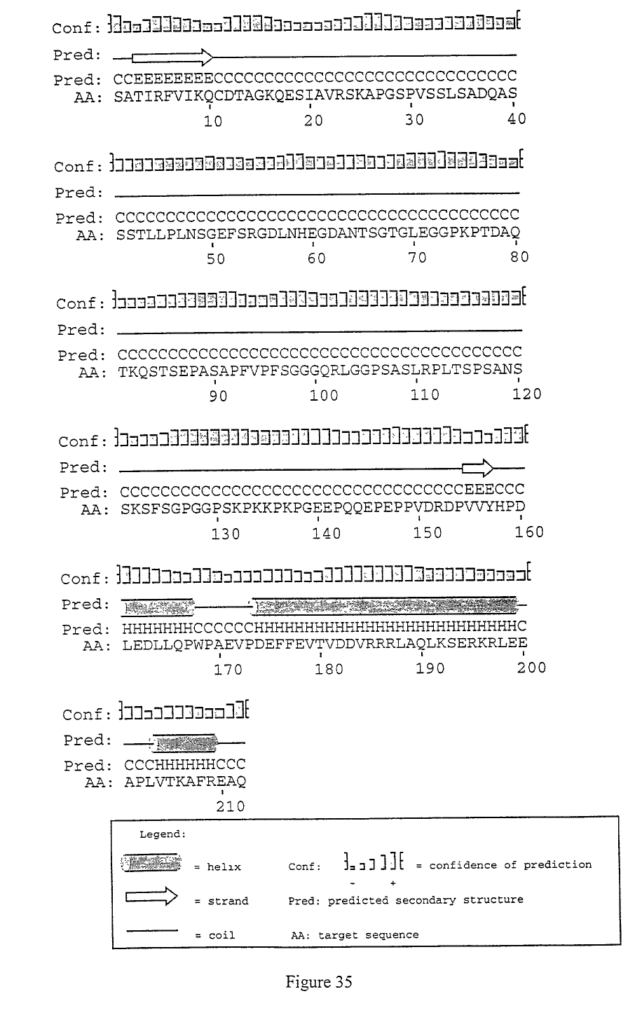

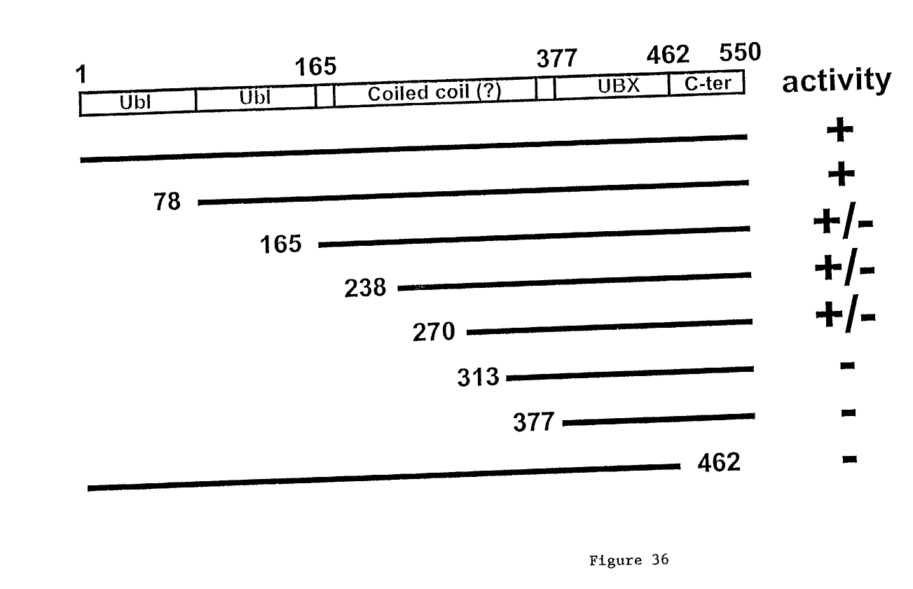

Flow cytometry was done on FACScan or FACSCalibur cytometers (Becton-Dickinson). Appropriate compensation between the FL1 and FL2 channels was set using uninfected (GFP-negative) cells or cells stained with PE only (e.g. using a PE-conjugated anti-transferrin receptor antibody; Pharmingen). Pilot experiments demonstrated minimal loss of viability; only 2% to 3% of the cells typically stained with propidium iodide using the protocol described above, so propidium iodide was not used in experiments where accurate compensation and quantitation of fluorescence intensities was essential. For each sample, data from ≧10,000 cells were collected. Median fluorescence intensities were used for quantification, since this measure of central tendency is least sensitive to outliers. For each sample, the PE and GFP fluorescence specifically attributable to the presence of the GLUT4 reporter were determined by subtracting background fluorescences, measured using control unstained cells and cells not expressing the reporter, respectively. These control cells were treated with the same conditions (e.g., type of serum and media, amino acid concentrations) used for the experimental cells. The ratio of fluorescence intensities plotted on the vertical axes of many figures is a relative, not absolute, measure of the proportion of GLUT4 at the cell surface. The scales are numbered arbitrarily and are not intended to permit comparison in absolute terms of data obtained in different experiments. In some of the kinetic studies (FIG. 26), the data were subjected to a simple smoothing operation. This consisted of calculating (0.25rₐ₁_{_{—}}0.5r_{c}+0.25r_{c+1}), where r is the ratio of specific fluorescences for timepoint t. This calculated value was used for timepoints t=2 to ten−1, where n is the total number of timepoints. For the last time point t=n, (0.33r_{c1}+0.67r₁) was calculated. Basal values of r (r₁) were unchanged.

Subcellular Fractionation

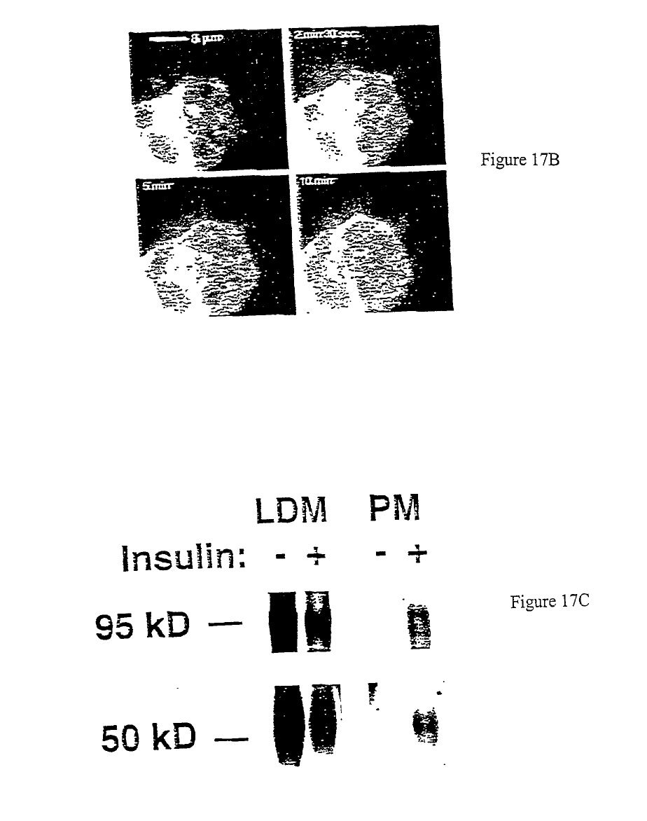

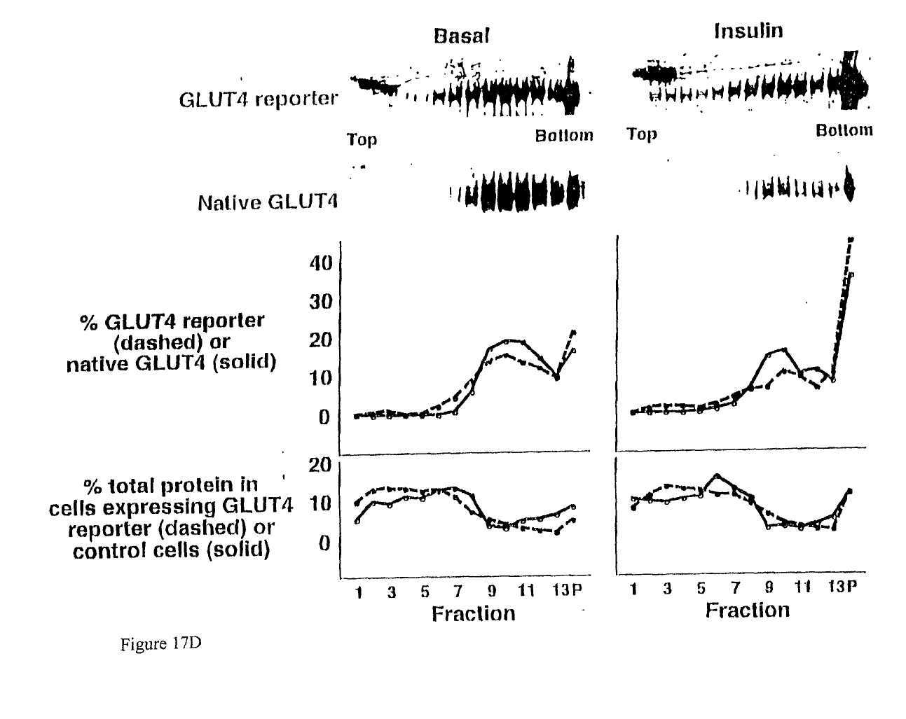

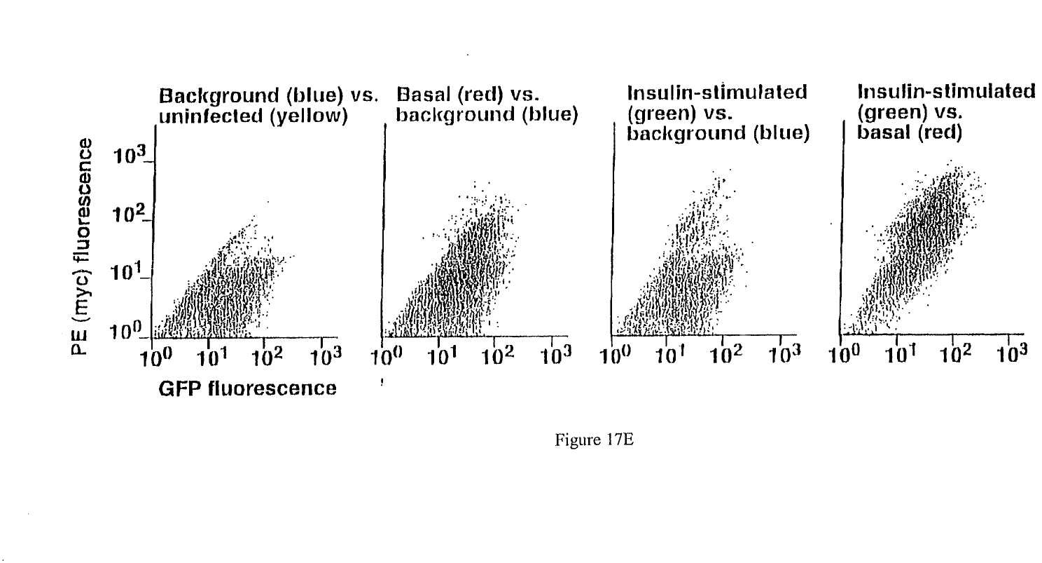

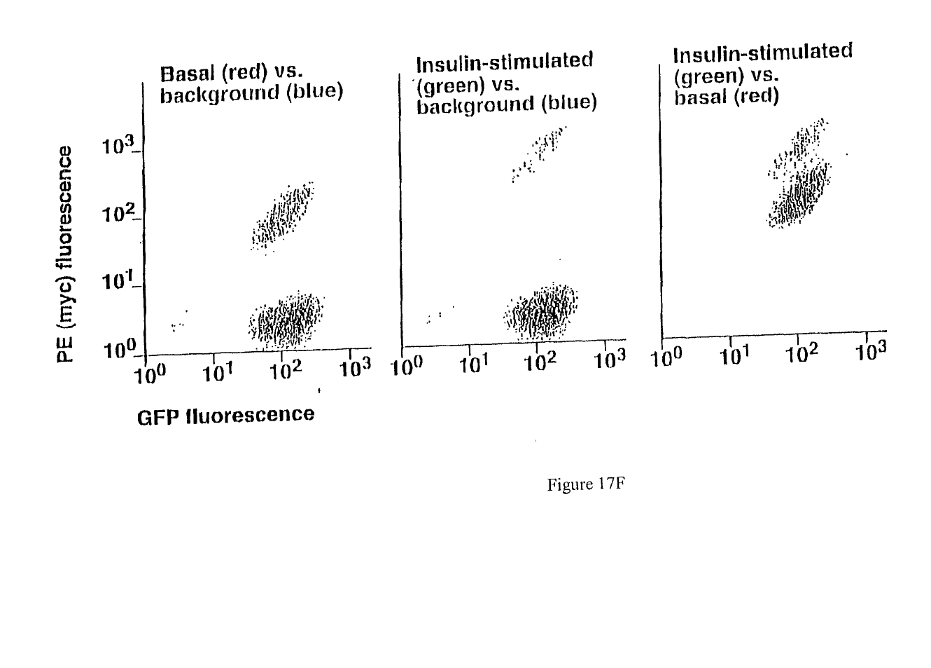

Four 10 cm plates of 3T3-L1 adipocytes were used per condition to isolate low density microsomal and plasma membrane fractions for FIG. 17B. Eight and ten plates per condition were used to isolate low density microsomes for analysis by sucrose density gradient centrifugation (FIG. 17C) and in vesicle immunopurification experiments (FIG. 17D), respectively. Five plates of adipocytes and ten plates of preadipocytes were used per condition in experiments presented in FIGS. 19B and 20B, respectively. For experiments presented in FIGS. 17B and 17D cells were serum starved overnight, treated with the presence or absence of insulin (160 nM, 10 min.), then transferred to 4° C. and washed with cold PBS++. For the vesicle immunopurification (FIG. 17D) cells were starved overnight, then transferred to 4° C. and washed with cold PBS++ without insulin stimulation. For kinetic studies (FIGS. 19B), 480 nM insulin was used (added from a prewarmed 3×stock) in order to maximally and simultaneously stimulate all of the cells on each 10 cm dish. Cell were transferred to 4° C. and washed with cold PBS++ at the indicated times. In all cases, cells were next washed once with cold 250 mM sucrose, 10 mM Tris pH 7.4, 0.5 mM EDTA (Buffer A). Cells were resuspended by scraping in cold Buffer A with Complete protease inhibitors (Roche), then homogenized using 16 strokes (4 plates) or 25 strokes (8 plates) in a Dounce-type teflon tissue grinder (Kontes #22, VWR). All subsequent steps were performed at 4° C. The homogenate was centrifuged at 11,500 rpm in an Ss-24 rotor (16,000×g) for 20 minutes. The pellet was resuspended in Buffer A, then layered on tope of 1.12M sucrose, 10 mM Tris pH 7.4, 0.5 mM EDTA in a ˜2 ml centrifuge tube. The samples were centrifuged in a TLS-55 rotor at 36,000 rpm (158,000×g) for 20 minutes. The interface was removed using a syringe, diluted in Buffer A, and centrifuged in a TLA-100.2 rotor at 37,000 rpm (74,000×g) for 9 minutes. The pellet was resuspended in Buffer A and centrifuged again under identical conditions. The pellet from this centrifugation, designated “PM”, was resuspended in TNET (1% Triton X-100, 150 mM NaCl, 20 mM Tris pH 8.0, 2 mM EDTA) or in RIPA buffer (PBS, 1% NP-40, 0.5% sodium deoxycholate, 0.1% SDS) and was stored at −20° C. until needed.

The supernatant from the initial centrifugation was recentrifuged at 19,000 rpm in an SS-34 rotor (43,000×g) for 30 minutes. The pellet (designated “HDM”) was resuspended in TNET or RIPA buffer and stored at −20° C. The supernatant was centrifuged at 65,000 in a Ti 70.1 rotor (39,000×g) for 75 minutes. The pellets from this centrifugation, designated “LDM”, were either resuspended in TNET or RIPA and stored at 20° C. until needed, or were resuspended in 500 μl 50 mM sucrose, 10 mM Tris pH 7.4, 0.5 mM EDTA and loaded on top of a 4.9 ml 10-30% (wt/vol) linear sucrose gradient prepared in 10 mM Tris pH 7.4, 0.5 mM EDTA. Separation of low density microsomes by sedimentation was essentially as described, with minor modifications (Czech, M. P., et al., 1993, J Cell Biol 123:127-35). The samples were centrifuged in a SW50.1 rotor at 48,000 rpm (280,000×g) for 55 minutes. Fractions were collected from the tops of the gradients. Equal volumes of each fraction were analyzed by SDS-PAGE and immunoblotting, and the total protein content in each fraction was determined using the micro-BCA kit (Pierce). In experiments where LDM and PM fractions were analyzed directly (without sedimentation of the LDM), total protein content was first determined and then equal amounts of protein were analyzed by SDS-PAGE and immunoblotting.

For vesicle immunopurification experiments, LDM fractions were resuspended in PBS++, 2% BSA, then incubated overnight at 4° C. in the presence of 25 μl (10 μg) anti-GFP monoclonal antibodies (Roche). Protein G sepharose beads were preblocked with PBS++, 2% BSA, then added to each sample and incubated for Ihour at 4° C. The beads were pelleted and the supernatant was transferred to new tubes and frozen until needed. The beads were washed five times in PBS++, 2% BSA, then three more times in PBS++ without BSA. Material was eluted from the beads at 65° C. for 30 minutes in SDS-PAGE sample buffer, and equal volumes were loaded for electrophoresis and subsequent immunoblotting.

Other Methods

Immunofluorescence microscopy of permeablized cells was done essentially as described (Bogan, J. S., and H. F. Lodish, 1999, J Cell Biol 146:609-20). Cells were fixed with 4% paraformaldehyde in PBS for 40 minutes, permeablized with 0.2% Triton X-100 for 5 minutes, then washed extensively with PBS++. Staining of CHO cells expressing the GLUT4 reporter was done in order to increase the fluorescent signal from that due to GFP alone, and employed a monoclonal anti-myc antibody and a FITC-conjugated secondary antibody.

For immunofluorescence microscopy of unpermeablized cells on coverslips were stimulated with insulin, then transferred to 4° C. and washed with cold PBS++. Living cells were stained at 4° C. using 25 μg/ml anti-myc (9E10) monoclonal antibody in PBS++, 2% BSA, 4% goat serum. After lhour at 4° C., cells were washed and incubated with 10 μg/ml Alexa594-conjugated goat anti-mouse IgG secondary antibody (Molecular Probes). Cells were then fixed with parafomaldehyde as described above, and coverslips were mounted using Prolong anti-fade reagent (Molecular Probes). Microscopy was done using a Zeiss Axiophot microscope, and images were acquired on film. In order to compare fluorescence intensity due to externalized myc epitope, GFP images were acquired first using an exposure time calculated by the camera, and the exposure time used for the corresponding Alexa594 images was set as a constant fraction of the GFP exposure time. In this way, the images of externalized myc epitope tag was normalized for variations in the total amount of the reporter protein and cell density.

Oil Red staining was done on cells grown in 10 cm dishes. Cells were fixed with 4% paraformaldehyde for 45 minutes at room temperature, permeablized with 0.2% Triton X-100 for 5 minutes at 4° C., and stained using a 2 mg/ml solution of Oil Red O in ethanol (Green, H., et al. 1975 Cell 5:19-27). Phase contrast and brightfield microscopy was done using an Olympus inverted microscope.

### EXAMPLE 1

### A Novel Assay for GLUT4 Trafficking at the Plasma Membrane

To assay changes in the proportion of GLUT4 present at the plasma membrane, a cDNA encoding a GLUT4 reporter protein was constructed. This protein contains seven c-myc epitope tags in the first exofacial loop of GLUT4, and GFP fused in-frame at the carboxy terminus. As shown in FIG. 17A, expression of this protein in cells enables the measurement of changes in the proportion of GLUT4 at the cell surface as changes in a ratio of fluorescence intensities. An anti-myc monoclonal antibody, followed by a phencoerythrin (PE)-conjugated secondary antibody, was used to detect reporter protein present at the surface of living cells. GFP fluorescence indicates the total amount of the reporter present in each cell. Thus, the ratio of PE to GFP fluorescence intensities corresponds to the proportion of total GLUT4 that is present at the plasma membrane. Flow cytometry was employed to measure these fluorescence intensities simultaneously and on a cell-by-cell basis.

The GLUT4 reporter was placed in a murine retroviral vector and 3T3-L1 preadipocytes were infected. Using fluorescence activated cell sorting (FACS), a population of cells falling within with a narrow range of GFP fluorescence intensities was isolated; individual cells in this population express similar amounts of the reporter protein. These 3T3-L1 cells underwent normal adipose differentiation (see below), and several approaches were taken to confirm that the GLUT4 reporter traffics appropriately. First, differential centrifugation was used to isolate low density microsomal (LDM) and plasma membrane (PM) fractions from cells expressing the GLUT4myc7-GFP reporter or from control cells. As shown in FIG. 17B, acute insulin treatment causes similar decreases in the amounts of both native GLUT4 (˜50 kD) and the reporter protein (˜95 kD) in the LDM fraction. Correspondingly, insulin treatment causes similar increases of both proteins in the PM fraction. Of note, the increases in the plasma membrane fraction are in the 5- to 10-fold range (insulin/basal), as judged by densitometry of several experiments. Thus, both the reporter and endogenous GLUT4 protein redistribute to the plasma membrane in a quantitatively similar manner.

FIG. 18C presents data further substantiating that the GLUT4 reporter codistributes with endogenous GLUT4 in low density microsomes prepared from 3T3-L1 adipocytes. In this experiment, LDM fractions were first isolated from basal and insulin stimulated cells containing the reporter and from control cells not expressing the reporter. These fractions were layered on top of 10-30% linear sucrose density gradients, and the microsomes were sedimented by centrifugation. Fractions from each sample were then collected and immunoblotted to detect endogenous GLUT4 or the reporter. As shown in the upper left panels of FIG. 17C, both native GLUT4 and the reporter sediment similarly in unstimulated cells, with the bulk of both proteins present in fractions 8-13 and in the pellet (middle left panel). In insulin-stimulated cells, the distributions of both endogenous GLUT4 and the reporter shift to the pellet; fractions 8-13 contain most of the remaining protein, as in the unstimulated cells (top and middle right panels). As a control, total protein was measured in each fraction and is plotted at the bottom of the figure for unstimulated cells (left) and for insulin stimulated cells (right). These profiles are similar for cells containing the GLUT4 reporter and for control cells, and are distinct from the distributions of endogenous GLUT4 and the GLUT4 reporter. The distribution of the GLUT4 reporter on these gradients is broader than that of native GLUT4, perhaps because the reporter is expressed at roughly five-fold higher levels (not shown), and may be present in a more heterogeneous population of vesicles. Nonetheless, the bulk of GLUT4 reporter in low density microsomes is present in vesicles that sediment similarly to those containing native GLUT4, both in basal and insulin-stimulated cells.

To further determine if endogenous GLUT4 and the GLUT4 reporter are present in the same vesicles, an anti-GFP antibody and protein G sepharose beads were used to immunopurify vesicles from the LDM fraction of unstimulated 3T3-L1 adipocytes expressing the reporter. As shown in FIG. 17D, immunoblotting demonstrates the presence of endogenous GLUT4 in these vesicles, as well as confirming the presence of the reporter. As a control, 3T3-L1 adipocytes not expressing the GLUT4 reporter were treated in parallel; in this case neither the GLUT4 reporter nor endogenous GLUT4 was detected in the material eluted from the beads. Endogenous GLUT4 was detected in the supernatants from both samples, and as expected was depleted from that of the cells expressing the reporter. Some GLUT4 reporter also remained in the supernatant, and it is estimated that the immunopurification removed only 50-75% of vesicles containing the reporter protein from the starting microsomes. The results indicate that endogenous GLUT4 and the GLUT4 reporter are present together in a population of vesicles within the low density microsomal fraction.

To measure insulin-stimulated GLUT4 externalization in differentiated 3T3-L1 adipocytes expressing the reporter, flow cytometry as shown in FIG. 17E was used. This technique allows simultaneous measurement of PE fluorescence (corresponding to cell-surface GLUT4 reporter and shown on the vertical scale) and GFP fluorescence (corresponding to total GLUT4 reporter and shown on the horizontal scale). Control 3T3-L1 adipocytes not expressing the reporter (shown in yellow) have background fluorescences that are highly correlated across the relevant wavelengths, and appear as a diagonal population. For cells expressing the reporter but not stained for cell-surface myc epitope (shown in blue), fluorescence along the PE axis is due entirely to this background autofluorescence. Control experiments using secondary antibody without primary (anti-myc) antibody, as well as control experiments using both primary and secondary antibodies on cells not expressing the reporter, demonstrate that background staining is negligible (data not shown); thus essentially all of the increase in PE fluorescence observed in the basal (red) and insulin-stimulated (green) populations is due to detection of myc on the cell surface. Similarly, the GFP fluorescence attributable to the GLUT4 reporter can be determined by subtracting the background autofluorescence (yellow). Within each population of stained cells (basal and insulin-stimulated), the amount of staining for cell-surface myc correlates with the amount of the reporter present, and therefore with GFP fluorescence. The populations therefore lie along a diagonal, and GLUT4 exocytosis results in a net translocation of the entire population upwards, along the PE axis, with no change in the slope of the diagonal. Within this defined population of cells no saturation of the recycling mechanism was observed: changes in the proportion of GLUT4 at the cell surface were equivalent, even among cells expressing ˜50-fold different amounts of the reporter.

To measure GLUT4 trafficking at the surface of CHO cells, CHO cells expressing the murine ecotropic retroviral receptor were infected with a retrovirus carrying the GLUT4 reporter, and FACS was utilized to isolate cells falling within a narrow range of GFP fluorescence intensities. Upon insulin stimulation of these cells, externalization of the GLUT4 reporter was again noted, as detected by flow cytometry. As shown in FIG. 17F, autofluorescence accounts for much less of the total fluorescent signals in CHO compared to 3T3-L1. Thus, the unstained cells expressing the reporter do not fall along a diagonal because autofluorescence contributes minimally. Similarly, whereas autofluorescence contributes perhaps one-quarter of the total PE fluorescence in 3T3-L1 adipocytes, in CHO cells, this figure is reduced to 1-2%. As with 3T3-L1 adipotytes, the distribution of each of the stained populations (basal and insulin-stimulated, shown in red and green, respectively) falls along a diagonal because the amount of myc epitope at the surface of each cell is proportional to the amount of the reporter present within that cell. Insulin stimulates the entire population to shift upward (˜5-fold) along the PE (myc) axis, with no change in the slope of the diagonal, consistent with exocytosis of the GLUT4 reporter equally and with no saturation of the recycling mechanism among the infected cells.

### EXAMPLE 2

### Insulin Stimulates GLUT4 Translocation Similarly in Undifferentiated 3T3-L1 Cells and Throughout 3T3-L1 Adipose Differentiation

One difficulty in working with 3T3-L1 cells is that if the undifferentiated fibroblasts are allowed to become confluent, they must either be induced to undergo adipose differentiation or they will lose that capacity. This characteristic makes the introduction of exogenous proteins by stable transfection technically difficult, since the cells invariably become confluent during clonal selection. This difficulty was circumvented by isolating a pool of cells infected with a replication-deficient retrovirus encoding the reporter protein. Over 90% of the target 3T3-L1 fibroblasts were infected, and those falling within a narrow window of GFP fluorescence were isolated by FACS; individual cells in this sorted population express similar amounts of the reporter. FIG. 18A demonstrates that these sorted cells undergo normal 3T3-L1 adipose differentiation, as assessed by Oil red and O staining to highlight the development of intracellular lipid droplets.

It was important to determine at what point, during the course of 3T3-L1 adipocyte differentiation, the cells become competent to translocate GLUT4 to the plasma membrane after insulin addition. Cells expressing the reporter were differentiated to different days, and the ability of insulin to stimulate GLUT4 exocytosis was tested as described above. Unexpectedly, as shown in FIG. 18B (and subsequently in FIG. 19A), it was found that insulin stimulates GLUT4 exocytosis at all times during 3T3-L1 differentiation, even in undifferentiated, confluent fibroblasts. Moreover, as shown in FIG. 18B, insulin-triggered GLUT4 exocytosis was invariably blocked by pretreatment of the cells with phosphatidylinositol-3-kinase inhibitors, either Wortmannin or LY294002. In several experiments it was noted that the overall foldincrease in cell-surface GLUT4 was greater at Day 2 of differentiation than at Day 0, primarily due to more pronounced intracellular sequestration in unstimulated cells; nonetheless the effect at Day 0is robust and consistently observed (FIG. 18B).

To determine if the kinetics of insulin-stimulated GLUT4 externalization vary during the course of 3T3-L1 differentiation, cells were stimulated at different days of differentiation and changes in the proportion of GLUT4 at the cell surface were assayed as a function of time. As shown in FIG. 19A, insulin triggers a rapid redistribution of GLUT4 to the cell surface, with identical kinetics at all days of 3T3-L1 differentiation. In all cases there is a biphasic response to insulin addition, with an initial rapid externalization of GLUT4 such that the greatest proportion is present on the plasma membrane four to five minutes after insulin addition. Subsequently, in all cases, the fraction of GLUT4 on the plasma membrane falls by 20% to 40%, and reaches a steady-state by 15 to 20 minutes after insulin addition. This “overshoot” of the steady-state proportion of GLUT4 at the cell surface in the presence of insulin presumably corresponds to rapid mobilization (and depletion) of GLUT4 in a highly insulin-sensitive compartment.

Because of the relative ease with which GLUT4 at the cell surface can be measured, all of the data points presented in all six panels of FIG. 19A were acquired in the same experiment. As noted above, the assay is internally controlled for the amount of reporter present within each cell. Thus, any data point in any of the six panels can be directly compared to any other data point in the figure. Clearly, unstimulated Day 2 cells have a lower proportion of GLUT4 on the cell surface than unstimulated confluent fibroblasts (Day 0 cells), in agreement with the data presented in FIG. 18B. While the slightly higher basal level of GLUT4 at the surface of confluent fibroblasts lessens the overall “fold-increase” in cell surface GLUT4 after insulin addition, the overall picture is similar in undifferentiated 3T3-L1 cells and in cells that have undergone any degree of adipose differentiation. Importantly, the overshoot of the final, steady-state response in the presence of insulin in present in all cases.

To determine if insulin stimulates externalization of native GLUT4 with a similar overshoot of the final, steady-state response, subcellular fractionation of 3T3-L1 adipocytes was performed. These cells do not express the GLUT4 reporter protein, and the presence of endogenous GLUT4 was detected by immunoblotting equal amounts of total protein from each time point. As shown in FIG. 19B, a rapid accumulation of GLUT4 in the plasma membrane fraction CM GLUT4) was observed, as was a corresponding decrease in GLUT4 present in the low density microsomal fraction (LDM GLUT4). The presence of GLUT4 in the PM fraction peaks at 7 minutes after insulin addition, subsequently there is a decrease in the amount present. Similarly, GLUT4 in the LDM fraction is first depleted, and subsequently reaccumulates slightly. As a control, the blot of the plasma membrane fractions was reprobed with an anti-insulin receptor β-chain antibody. As shown in the lower panel of the Figure, this detects similar amounts of insulin receptor at the plasma membrane at most time points. There is a slight decrease immediately (3 minutes) after insulin addition, perhaps due to internalization of the receptor, and the amount normalizes at subsequent times. Thus, immunoblotting of subcellular fractions demonstrates that native GLUT4 traffics with kinetics similar to those observed using the tagged GLUT4 reporter and the FACS-based assay.

Reinternalization and recycling of the GLUT4 reporter was examined after insulin removal. Based on the results shown in FIG. 19A, cells were stimulated with insulin for 20 minutes so that the redistribution of GLUT4 to the plasma membrane would be at steady-state, then chilled, washed with a low pH buffer to remove insulin, and rewarmed in serum-free medium for varying amounts of time. Cells were allowed to reinternalize GLUT4 for up to two hours, at which time they were restimulated with insulin for 5, 10, or 15 minutes. As shown in FIG. 19C, the reporter protein was reinternalized in undifferentiated 3T3-L1 cells and at all times of 3T3-L1 adipocyte differentiation, and was recycled upon restimulation with insulin in all cases. All of the data points in FIG. 19C were collected in parallel and can be compared, even if presented in different panels of the figure. The rate of reinternalization is slightly prolonged in more differentiated cells as compared to less differentiated cells. Finally, restimulation with insulin causes reexternalization of the reporter; the magnitude and kinetics of this effect is similar to the initial response, and the biphasic pattern described above is likely present. It can be concluded that the addition of myc epitope tags and fusion of GFP to the carboxy terminus of GLUT4 does not impair its ability to undergo endocytosis or insulin-stimulated recycling at the plasma membrane.

As noted above, the observation that insulin-stimulated GLUT4 translocation to the plasma membrane of undifferentiated 3T3-L1 preadipocytes was unexpected. To determine if this response was general to other cell types, the GLUT4 reporter protein was expressed in NIH 3T3 cells by retroviral infection, a stable pool of cells was isolated by flow sorting, and insulin-stimulated externalization was compared in these cells and in 3T3-L1 preadipocytes. As shown in FIG. 19A, the NIH 3T3 cells respond poorly to insulin stimulation, with less than a twofold increase in the proportion of GLUT4 at the cell surface. In contrast, the 3T3-L1 preadipocytes demonstrate a rapid externalization, such that the increase in cell-surface GLUT4 reached almost fourfold at 5 minutes after insulin addition. Subsequently, the proportion of GLUT4 at the cell surface decreases markedly, so that the first phase of the response overshoots the final steady-state. The data are consistent with the presence of a highly insulin-responsive pool of GLUT4 in the basal state in 3T3-L1 preadipocytes, but not in NIH 3T3 cells. After the initial, rapid mobilization (and depletion) of this pool, the 3T3-L1 preadipocytes may be only marginally able to recycle GLUT4 faster than NIH 3T3 cells, so that the difference in steady-state presence of insulin is minimal. The GLUT4 reporter was also expressed in a cultured, nontransformed hepatocyte cell line (AML12, Wu, J. C., et al., 1994 PNAS USA 91:674-8.) and found that in these cells as well insulin stimulated minimal translocation and there was no overshoot (not shown). It can be concluded that insulin regulates GLUT4 recycling through a highly insulin-responsive mechanism present in 3T3-L1 adipocytes and, at least to some degree, in 3T3-L1 preadipocytes, but that that is not characteristic of all cell types.

To further demonstrate that insulin mobilizes GLUT4 to the plasma membrane of 3T3-L1 preadipocytes, subcellular fractionation and immunoblotting was performed. Since native GLUT4 is not expressed in undifferentiated 3T3-L1 cells, cells expressing the GLUT4 reporter were utilized. As shown in FIG. 20, insulin stimulates movement of the GLUT4 reporter out of the LDM fraction and into the PM fraction. The peak response is at 8 minutes after insulin addition. By 20 minutes there is a decrease from this peak in the PM fraction, as well as a slight increase in the amount present in the LDM fraction. These subcellular fractionation data not only indicate that the GLUT4 reporter is translocated in 3T3-L1 preadipocytes, but also suggest that there is an early overshoot before the final steady-state response. Thus, the subcellular fractionation data are in accord with our flow cytometry data.

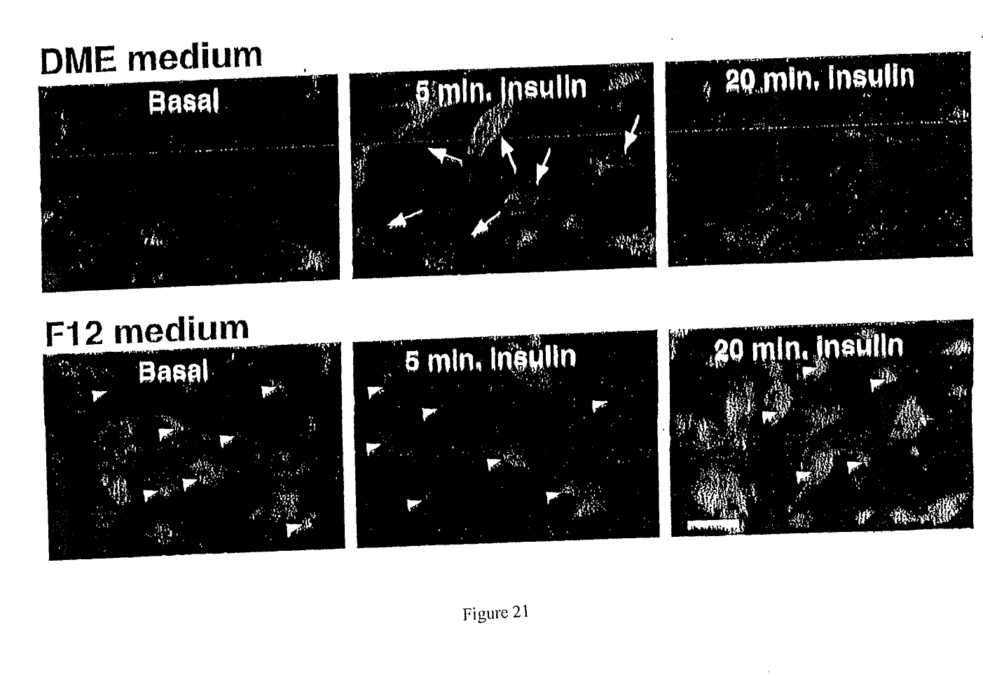

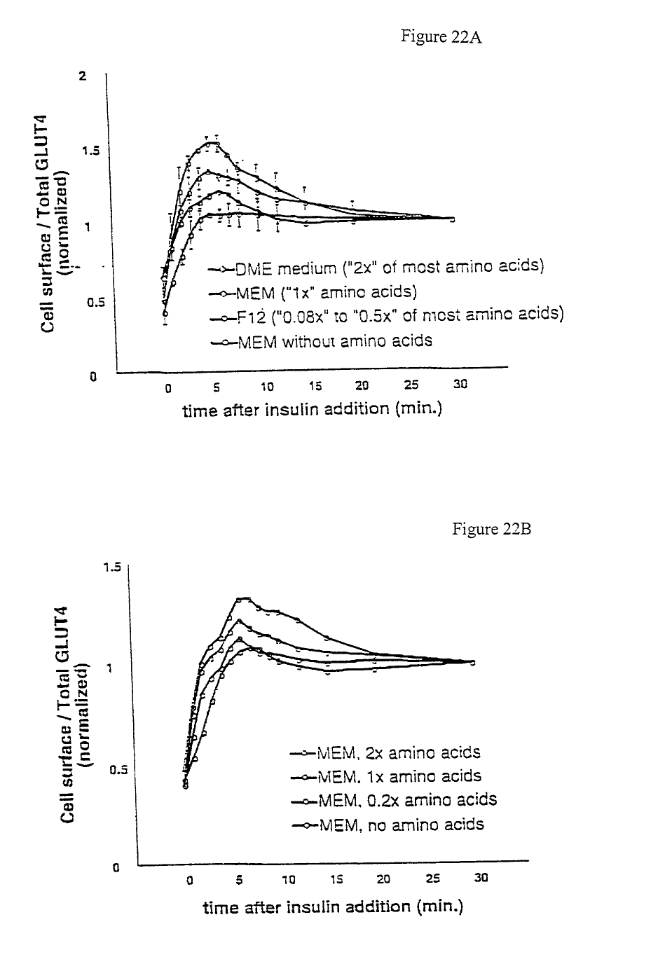

One possible explanation for the observation that GLUT4 translocation was noted in 3T3-L1 preadiopcytes in these experiments, whereas other researchers have not previously observed such translocation, is that these cells were routinely cultured in 10% fetal bovine serum rather than the more usual 10% calf serum. Confluent 3T3-L1 preadipocytes were cultured in each of these sera for three days prior to assaying the effect of insulin on GLUT4 trafficking. As shown in FIG. 21, cells cultured in 10% calf serum and in 10% fetal bovine serum responded indistinguishably. In both cases, insulin causes a four-fold increase in the fraction of GLUT4 present at the plasma membrane at early (5 minutes) time points, with a subsequent decrease as previously noted. These data are consistent with the those presented in FIGS. 20A and 21, and indicate that the effect observed does not result from the culture of 3T3-L1 preadipocytes in fetal bovine serum.

### EXAMPLE 3

### The Kinetics of Insulin Stimulated GLUT4 Translocation in CHO Cells are Media-dependent

To determine if the kinetics of insulin stimulated GLUT4 externalization were similar in CHO cells and in 3T3-L1 cells, changes in the proportion of GLUT4 at the cell surface of CHO cells stimulated for various amounts of time were assayed. To parallel the conditions used for 3T3-L1 cells, some CHO cells were placed in DMEM two days before the experiment; others were left in standard CHO medium (F12). As shown in FIG. 20, CHO cells cultured in DMEM respond to insulin with a dramatic, biphasic redistribution of GLUT4 to the cell surface. In contrast, CHO cells cultured in F12 medium redistributed GLUT4 less dramatically, and with no overshoot of the final, steady-state proportion of GLUT4 at the cell surface in the presence of insulin. As with 3T3-L1 cells, GLUT4 externalization in DMEM cultured CHO cells peaks at four to five minutes after insulin addition, and then decreases to reach a steady-state by twenty minutes after insulin addition. The peak fraction of GLUT4 at the cell surface is 5.5-fold more than that in unstimulated cells, and in several experiments 50% to 60% of this increase is eliminated in the subsequent decrease. For comparison, the peak response of 3T3-L1 adipocytes in FIG. 19A was 5.4-fold over basal (average of Day 8 and 10), though the subsequent decrease to steady-state was only ˜20% of this peak response.

To examine the subcellular distribution of the GLUT4 reporter in CHO cells cultured in these two distinct media, immunofluorescence microscopy was performed. Cells were cultured in F12 or DMEM for two days, stimulated with insulin for 0, 5, or 20 min, then chilled and stained without permeablization to detect the externalized myc epitope tag. As shown in FIG. 21, cells cultured in F12 have a small increase in the amount of externalized myc epitope at 5 minutes and significantly more at 20 minutes after insulin addition (top row, red images). In contrast, cells cultured in DMEM have greater cell-surface staining at 5 minutes than at 20 minutes after insulin addition (third row, red images); after 5 minutes of insulin treatment the amount of surface myc-GLUT4 fluorescence is much greater in cells cultured in DMEM than in F12. The distribution of GLUT4 reporter within the cells was also examined using GFP fluorescence. In this case, it was noted that cells cultured in F12 have an abundance of the reporter protein in the perinuclear region, both in the absence of insulin and after 5 or 20 minutes of insulin treatment (second row, green images). In cells cultured in DMEM, this perinuclear accumulation is less marked (fourth row, green images). Though these microscopy data are more difficult to accurately quantify than the flow cytometry data (FIG. 18), it is clear that the kinetics observed by microscopy are similar to those observed using flow cytometry.

FIG. 21 presents a higher magnification demonstrating the intracellular distribution of the GLUT4 reporter in CHO cells cultured in these two distinct media. In the basal state, GLUT4 is prominent in the perinuclear region in cells cultured in F12 medium (FIG. 21, lower left panel, arrowheads). In contrast, cells cultured for two days in DMEM have less GLUT4 in the perinuclear region, and more that is present in punctate structures in the periphery (upper left panel). Insulin treatment for 5 minutes causes a dramatic increase in plasma membrane GLUT4 in cells cultured in DMEM, (FIG. 21 arrows, upper center panel). Cells cultured in F12 have a less marked accumulation of GLUT4 at the plasma membrane after 5 minutes insulin treatment (FIG. 21 lower center panel). By 20 minutes after insulin addition, the amount of GLUT4 at the plasma membrane is similar in cells cultured in both media, and is less than the peak response in 5 minutes insulin treatment for cells cultured in DMEM (right panels of FIG. 21). Of note, cells cultured in F12 medium have continued prominent perinuclear GLUT4 accumulation even after 5 or 20 minutes insulin treatment (FIG. 21 arrowheads, lower center and right panels). These data are consistent with the flow cytometry data presented in FIG. 18. Thus, correlation of kinetic and microscopy data suggests that GLUT4 accumulates in a peripheral, highly insulin-responsive compartment in the basal state when the cells are cultured in DMEM. The perinuclear GLUT4 accumulation seen in the cells cultured in F12 may represent a longer term reservoir. Finally, the overshoot of the steady-state proportion of GLUT4 at the cell surface in the presence of insulin corresponds, to a first approximation, to the amount of GLUT4 that has accumulated in the peripheral compartment in CHO cells.

### EXAMPLE 4

### Amino Acid Sufficiency Modulates Highly Insulin-Responsive GLUT4 Trafficking in CHO Cells and in 3T3-L1 Adipocytes

DMEM and F12 media differ in several respects. Though DMEM has greater glucose and calcium concentrations, neither of these components alone or in combination proved necessary or sufficient to cause highly insulin-responsive (i.e., biphasic) kinetics (data not shown). It was noted that many essential amino acids are present at markedly higher concentrations in DMEM than in F12 and the possibility that these are required for highly insulin-responsive GLUT4 trafficking was tested. After culture for 24-36 hours in various media, the kinetics of insulin-stimulated GLUT4 translocation in CHO cells was examined. As shown in FIG. 22A, the degree of overshoot of the final steady-state response in the presence of insulin correlates quite well with the concentration of most essential amino acids in different media. DMEM has twofold the concentrations of most amino acids present in MEM, which in turn has 2- to 12-fold greater concentrations of most amino acids compared to F12. In MEM made without any amino acids, no overshoot of the final, steady-state proportion of GLUT4 at the plasma membrane in the presence of insulin was observed. The kinetics of GLUT4 externalization in CHO cells cultured in MEM with 2×, 1×, 0.2× or no amino acids was also tested. Higher concentrations of amino acids result in a greater overshoot of the final, steady-state response in the presence of insulin. Since glutamine was held constant, it can be surmised that flux through the glucosamine pathway is not likely to be responsible for this effect.

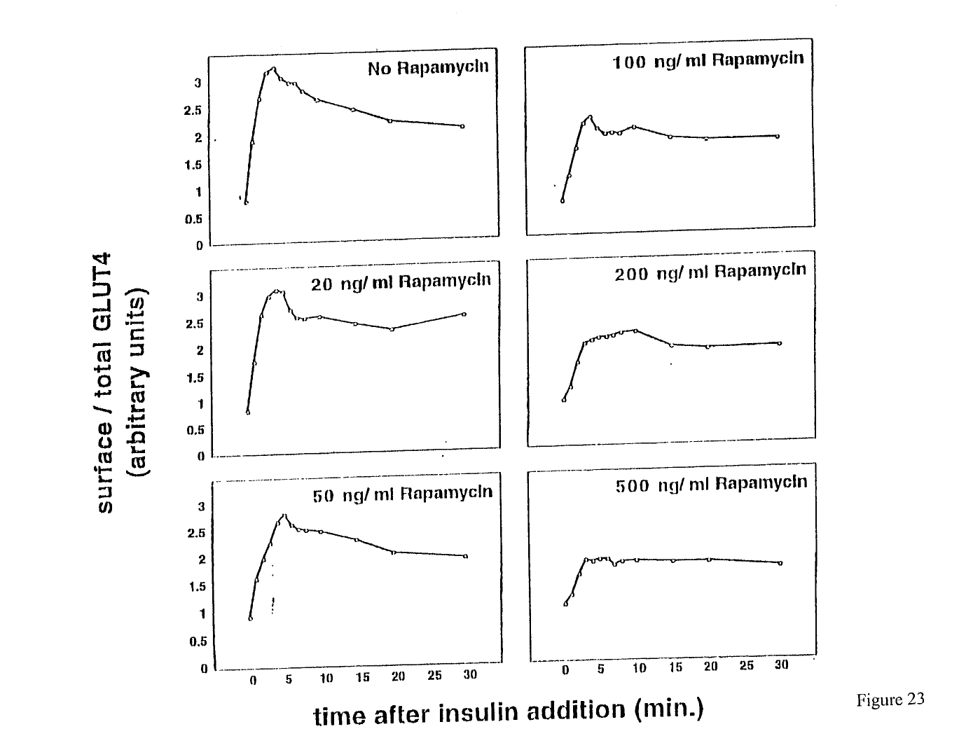

Another insulin signaling output that is sensitive to amino acid availability is the phosphorylation of p70 S6-kinase (Hara, K., et al., 1998 J Biol Chem 273:(34) 2216058). In this case, withdrawal of most individual amino acids inhibits the ability of insulin to stimulate p70 phosphorylation to various degrees; the most potent were leucine and arginine, and the effect is mimicked by rapamycin. A modest decrease in the proportion of GLUT4 rapidly mobilized by insulin in CHO cells cultured without leucine and arginine was also observed (not shown). More striking is the ability of rapamycin to alter the kinetics of insulin-stimulated GLUT4 externalization. As shown in FIG. 23, rapamycin used over a range of concentrations progressively eliminated the rapid, first phase of insulin stimulated GLUT4 externalization, such that at the highest concentration there is no overshoot of the final, steady-state response. Of note, the amount of GLUT4 reporter protein, as assessed by GFP fluorescence, did not change by more than ˜10% after amino acid deprivation or rapamycin treatment (not shown). These data parallel those that indicate that amino acid sufficiency modulates GLUT4 targeting to a highly insulin-responsive compartment through a rapamycin-sensitive mechanism.

To learn whether the observations in CHO cells were relevant to GLUT4 trafficking in 3T3-L1 adipocytes, the amino acid concentrations in which fully differentiated 3T3-L1 adipocytes expressing the reporter were cultured were varied. After 36 hours, the externalization of GLUT4 after insulin addition was examined. As shown in FIG. 24, 3T3-L1 adipocytes cultured in MEM with 2×amino acids (relative to standard MEM, and similar to DMEM) mobilized GLUT4 similarly to cells cultured in DMEM (e.g., FIG. 19A). Strikingly, culture in progressively lower concentrations of amino acids resulted in decreased magnitude of GLUT4 translocation (FIG. 24). In all cases, the overshoot of the steady-state response remains intact, even in the absence of amino acids (except glutamine). Indeed, for 3T3-L1 adipocytes cultured in the absence of amino acids, the response demonstrates a marked overshoot followed by only a modest “fold increase” of GLUT4 at the cell surface in the steady-state; this is somewhat reminiscent of the response observed using CHO cells cultured in DMEM (FIG. 21). Culture of 3T3-L1 cells in the presence of rapamycin has a similar effect, as shown in FIG. 25. Here, a progressive increase in the concentration of rapamycin caused a progressive decrease in the magnitude of GLUT4 translocation by insulin. As with amino acid insufficiency, rapamycin treatment of 3T3-L1 adipocytes does not alter the presence of biphasic kinetics. Finally, the amount of GLUT4 reporter present in each cell, as assessed by GFP fluorescence, did not decrease by more than 10% after amino acid starvation or rapamycin treatment (not shown). Overall, the data are consistent with the concept that amino acid sufficiency modulates GLUT4 trafficking through a kinetically-defined, highly insulin-responsive compartment in 3T3-L1 adipocytes, and that this effect is rapamycin-sensitive.

While this invention has been particularly shown and described with references to particular embodiments thereof, it will be understood by those skilled in the art that various changes in form and details may be made therein without departing from the scope of the invention encompassed by the appended claims.

What is claimed is:

1. A method of enriching an expression library comprising DNA encoding a protein involved in insulin stimulated GLUT4 trafficking at the plasma membrane comprising:
a) sorting cells containing an expression library comprising DNA encoding a protein involved in insulin stimulated GLUT4 trafficking for cells with an altered proportion of GLUT4 at the cell surface;
b) expanding the sorted cells from step a) in culture;
c) sorting the cells expanded in step b); and
d) expanding the cells sorted in step c);
thereby forming an expression library enriched for DNA encoding a protein involved in GLUT4 trafficking at the plasma membrane.

2. The method of claim 1, wherein the cells containing the expression library respond to insulin stimulation by externalizing GLUT4 in a biphasic pattern.

3. The method of claim 1, wherein the cells with an altered proportion of GLUT4 at the cell surface are identified about five minutes after insulin stimulation.

4. The method of claim 1, wherein the proportion of GLUT4 at the cell surface is increased.

5. The method of claim 1, wherein the proportion of GLUT4 at the cell surface is decreased.

6. The method of claim 1, wherein the method comprises additional steps of sorting the cells and expanding the sorted cells in culture.

7. The method of claim 6, wherein in one sorting step individual cells are sorted for expansion.

8. The method of claim 1, wherein the cells were cultured in a media with high amino acid content.

9. The method of claim 8, wherein the media is Dulbecco's modified Eagle's medium (DMEM).

10. The method of claim 1, wherein the cells are selected from the group of adipocyte, fibroblast and muscle cells.

11. The method of claim 1, wherein the cells are 3T3-L1 or Chinese Hamster Ovary (CHO) cells.

12. The expression library prepared in the method of claim 1.

13. The expression library prepared in the method of claim 5.

14. The expression library prepared in the method of claim 6.

15. A method for identifying a protein involved in insulin stimulated GLUT4 trafficking at the plasma membrane comprising:
a) introducing an expression library comprising DNA encoding a protein involved in GLUT4 trafficking at the plasma membrane into a population of cells;
b) maintaining the population of cells under conditions suitable for replication of the DNA encoding a protein involved in GLUT4 trafficking at the plasma membrane and for isolating individual clones;
c) subdividing the population of cells into pools of cells such that each pool is a subset of the population of cells;
d) isolating the replicated DNA encoding a protein involved in GLUT4 trafficking at the plasma membrane from the pools of cells;
e) introducing the replicated DNA encoding a protein involved in GLUT4 trafficking at the plasma membrane into cells containing a GLUT4 reporter protein; and
(f) assessing trafficking of the reporter protein,
wherein altered trafficking of the reporter protein upon insulin stimulation is indicative of the presence of a protein involved in GLUT4 trafficking at the plasma membrane.

16. The method of claim 15, further comprising repeating steps c)-f) for the pools of cells containing a protein involved in GLUT4 trafficking at the plasma membrane.

17. The method of claim 15, wherein the cells containing the expression library respond to insulin stimulation by externalizing GLUT4 in a biphasic pattern.

18. The method of claim 15, wherein the protein is associated with an increased proportion of GLUT4 at the plasma membrane.

19. The method of claim 15, wherein the protein is associated with a decreased proportion of GLUT4 at the plasma membrane.

20. The method of claim 15, wherein the cells of the expression library are selected from the group of adipocyte, fibroblast and muscle cells.

21. The method of claim 15, wherein the cells of the expression library are 3T3-L1 or CHO cells.

22. A protein identified in the method of claim 15.

23. The protein of claim 22 comprising a UBX domain.

24. The protein of claim 22 comprising a sequence selected from the group consisting of SEQ ID NO: 1, SEQ ID NO: 2, SEQ ID NO: 3 and SEQ ID NO: 4.

25. A method for identifying a protein involved in GLUT4 trafficking at the plasma membrane comprising the following steps:
a) preparing an expression library enriched for DNA encoding a protein involved in GLUT4 trafficking at the plasma membrane in cells;
b) stimulating the cells with insulin; and
c) screening the cells for altered GLUT4 trafficking at the plasma membrane;
wherein altered GLUT4 trafficking upon insulin stimulation is indicative of the presence of a protein involved in GLUT4 trafficking at the plasma membrane.

26. The method of claim 25, wherein the cells respond to insulin stimulation by externalizing GLUT4 in a biphasic pattern.

27. The method of claim 25, wherein the cells are screened by identifying those cells with altered proportions of GLUT4 at the cell surface about five minutes after insulin stimulation.

28. The method of claim 25, wherein the expression library is enriched for DNA associated with an increased proportion of GLUT4 at the plasma membrane.

29. The method of claim 25, wherein the expression library is enriched for DNA associated with a decreased proportion of GLUT4 at the plasma membrane.

30. The method of claim 25, wherein the cells are selected from the group of adipocyte, fibroblast and muscle cells.

31. The method of claim 25, wherein the cells are 3T3-L1 or CHO cells.

32. A protein identified in the method of claim 25.

33. The protein of claim 32 comprising a UBX domain.

34. The protein of claim 32 comprising a sequence selected from the group consisting of SEQ ID NO: 1, SEQ ID NO: 2, SEQ ID NO: 3 and SEQ ID NO: 4.

35. An isolated antibody or antigen binding fragment thereof which specifically binds to a protein selected from the group consisting of SEQ ID NO: 1, SEQ ID NO: 2, SEQ ID NO: 3 and SEQ ID NO: 4.

36. The antibody of claim 35, wherein the antibody is a monoclonal antibody.

37. The antibody of claim 35, wherein the antibody is a polyclonal antibody.

38. A method of identifying an agent which alters insulin stimulated GLUT4 trafficking at the plasma membrane comprising the steps of:
a) maintaining test cells in which GLUT4 is transferred to the cell surface upon stimulation with insulin in the presence of an agent which binds to a UBX domain;
b) measuring the proportion of GLUT4 at the cell surface of the test cells after insulin stimulation; and
c) comparing the proportion of GLUT4 at the cell surface with the proportion of GLUT4 at the cell surface in suitable control cells after insulin stimulation,
wherein an altered proportion of GLUT4 at the cell surface in the test cells compared to the proportion of GLUT4 in the control cells is indicative of altered insulin stimulated GLUT4 trafficking at the plasma membrane.

39. The method of claim 38, wherein GLUT4 trafficking at the plasma membrane is increased in the presence of the agent.

40. The method of claim 38, wherein GLUT4 trafficking at the plasma membrane is decreased in the presence of the agent.

41. The method of claim 38, wherein the agent binds to a protein comprising a sequence selected from the group of SEQ ID NO: 1, SEQ ID NO: 2, SEQ ID NO: 3 and SEQ ID NO: 4.

42. The method of claim 38, wherein the agent binds to L1.

43. The method of claim 38, wherein the cells are selected from the group of adipocyte, fibroblast and muscle cells.

44. The method of claim 38, wherein the cells are 3T3-L1 or CHO cells.

45. A method for identifying an agent which binds to a protein comprising a UBX domain comprising the steps of:
a) isolating a protein comprising a UBX domain;
b) contacting an agent with the isolated protein under conditions suitable for binding of the agent to the isolated protein; and
c) detecting a resulting agent-protein complex,
wherein detection of an agent-protein complex is indicative of an agent which binds to the protein.

46. The method of claim 45, wherein the agent binds to a protein comprising a sequence selected from the group consisting of SEQ ID NO: 1, SEQ ID NO: 2, SEQ ID NO: 3 and SEQ ID NO: 4.

47. A method of identifying an agent which alters the interaction between GLUT4 and L1 comprising:
a) comprising combining GLUT4, L1 and an agent under conditions appropriate for interaction between GLUT4 and L1
b) determining the extent to which GLUT4 and L1 interact; and
c) comparing the extent of the GLUT4-L1 interaction in the presence of the agent with the extent of GLUT4-L1 interaction in the absence of the agent,
whereby if the extent of the GLUT4-L1 interaction differs significantly in the presence of the agent when compared to the interaction in the absence of the agent, then the candidate agent is one which alters interaction between GLUT4 and L1.

48. The method of claim 47, wherein the agent enhances the interaction.

49. The method of claim 47, wherein the agent inhibits the interaction.

50. A method of altering insulin stimulated GLUT4 trafficking comprising contacting an insulin responsive cell with rapamycin.

51. A method of altering insulin stimulated GLUT4 trafficking comprising contacting a cell with an agent which binds to the UBX domain of a protein selected from the selected from the group consisting of SEQ ID NO: 1, SEQ ID NO: 2, SEQ ID NO: 3 and SEQ ID NO: 4.

52. A method of inhibiting GLUT4 externalization comprising contacting a GLUT4/L1 complex with an agent that inhibits disassociation of the complex.

53. A method of enhancing GLUT4 externalization comprising contacting a GLUT4/L1 complex with an agent that enhances disassociation of the complex.

54. A method of enriching an expression library comprising DNA encoding a protein involved in insulin stimulated GLUT4 trafficking at the plasma membrane comprising sorting cells containing an expression library comprising DNA encoding a protein involved in insulin stimulated GLUT4 trafficking for cells with an altered proportion of GLUT4 at the cell surface, thereby forming an expression library enriched for DNA encoding a protein involved in GLUT4 trafficking at the plasma membrane.

55. The method of claim 54, wherein the sorted cells are expanded in culture.

56. The method of claim 55 further comprising additional steps of sorting expanded cells and expanding the sorted expanded cells in culture until a desired level of enrichment is achieved.

57. A method of enriching an expression library comprising DNA encoding a protein involved GLUT4 trafficking comprising:
a) sorting cells containing an expression library comprising DNA encoding a protein involved in insulin stimulated GLUT4 trafficking for cells with an altered proportion of GLUT4 at the cell surface;
b) expanding the sorted cells from step a) in culture;
c) sorting the cells expanded in step b); and
d) expanding the cells sorted in step c);
thereby forming an expression library enriched for DNA encoding a protein involved in GLUT4 trafficking.
